(19) United States
(12) Patent Application Publication
(10) Pub. No.: US 2023/0293772 A1
(43) Pub. Date: Sep. 21, 2023

(54) COLLAGEN-POLYMER SCAFFOLD DELIVERY SYSTEM FOR PERIODONTAL REPAIR AND REGENERATION

(71) Applicant: RVO 2.0, INC., D/B/A OPTICS MEDICAL, ALISO VIEJO, CA (US)

(72) Inventors: Gabriel N. Njikang, Orcutt, CA (US); Alan Ngoc Le, Lake Forest, CA (US); Beverly W. Lubit, Kinnelon, NJ (US)

(73) Assignee: RVO 2.0, INC., D/B/A OPTICS MEDICAL, ALISO VIEJO, CA (US)

(21) Appl. No.: 18/123,809

(22) Filed: Mar. 20, 2023

Related U.S. Application Data
(60) Provisional application No. 63/322,123, filed on Mar. 21, 2022.

(51) Int. Cl. A61L 27/38; A61L 27/56; A61L 27/58; A61L 27/26; A61L 27/52

(52) U.S. Cl. CPC A61L 27/3865; A61L 27/56; A61L 27/58; A61L 27/3834; A61L 27/26; A61L 27/52; A61L 2400/06; A61L 2300/414

(57) ABSTRACT

The present disclosure provides a hydrogel composition comprising an interpenetrating polymer network (IPN) containing a biopolymer, a first synthetic polymer and a second synthetic polymer in which a contained community of live human MSCs is embedded. The collagen polymer matrix described (a) allows the embedded cells to remain in place or to migrate over short distances; (b) allows diffusion of small molecules, particularly growth factors produced by the cells or provided as a supplement, and EVs released by the cells to support the recovery of periodontium tissue function following injury.

### CROSS-REFERENCE TO RELATED APPLICATIONS

This application claims the benefit of priority to U.S. Provisional Patent Application No. 63/322,123, filed Mar. 21, 2022, which is incorporated herein by reference in its entirety.

### BACKGROUND OF THE INVENTION

The periodontium is a complex structure that contains at least six distinct tissue types, including the gingival epithelium, the gingival connective tissue, the periodontal ligament (PDL), the tooth root surface cementum, the alveolar bone, and corresponding vasculature. The periodontium exhibits a typical “layer by layer” (LBL) structure comprising cementum, alveolar bone and periodontal ligament (PDL) [Liu, J, et al. Periodontal Bone-Ligament-cementum regeneration via scaffolds and stem cells. Cells (2019) 8: 537]. The cementum occurs as a thin acellular layer around the tooth root neck, with thicker cellular cementum covering the lower part of the tooth root up to the apex [Id., citing Foster B. L., et al. Central role of pyrophosphate in acellular cementum formation. PLoS ONE. 2012; 7:e38393; Matalová E., L et al. Stem Cell Biology and Tissue Engineering in Dental Sciences. Academic Press; Cambridge, Mass., USA: 2015. Development of Tooth and Associated Structures; pp. 335-346; Foster B. L., et al. Advances in defining regulators of cementum development and periodontal regeneration. Curr. Top. Dev. Biol. 2007; 78:47-126]. The PDL consists of highly organized fibers, which are perpendicularly inserted into the cementum coated tooth root and adjoining the alveolar bone, where their ends (Sharpey's fibers) insert into the mineralized tissues to stabilize the tooth root, transmit occlusal forces, and provide sensory function. PDL fibers connect the cementum on the tooth root surface to the alveolar bone and fix the tooth in the alveolar socket to attenuate occlusal stresses.

Periodontal disease or periodontitis is a chronic inflammatory disease that begins with a period of inflammation of the supportive tissues of the teeth and then progresses. It is a common cause of receding gums that can lead to tooth loss and other serious health complications. All of these tissues are affected during chronic inflammation. FIG. 1A-FIG. 1B is a schematic representation of the periodontium containing the intact bone-PDL-cementum (FIG. 1A) and damage to the periodontium as a result of disease (FIG. 1B), which leads to loss of multiple periodontal tissues surrounding and supporting the tooth. [Taken from Xu, X-Y, et al. Stem Cell Translational Med. (92019) 8: 392-403, FIG. 2].

Periodontitis is initiated by an imbalance that causes the accumulation of pathogenic bacteria and their lipopolysaccharides. The destruction of the supporting tissues of the tooth in periodontitis is mainly due to an exacerbated immune response of the host in susceptible individuals, which prevents the acute inflammation from being resolved. [Hernandez-Monjaraz, B. et al. Intl J. Mol. Sci. (2018) 19: 944]. In these cases, the accumulation of bacteria in the gingival sulcus causes the migration of polymorphonuclear neutrophils (PMNs) and monocytes. These cells, together with those of the gingival epithelium, secrete cytokines such as interleukin IL-1β, IL-6 tumor necrosis factor-alpha (TNF-α), and adhesion molecules such as endoglin and intercellular adhesion molecule 1 (ICAM-1), which increase the adhesion of PMNs and monocytes to endothelial cells and increase the permeability of the gingival capillaries, which leads to the accumulation of leukocytes in the infection zone. [Id., citing Ford, P J et al. Immunological differences and similarities between chronic periodontitis and aggressive periodontitis. Periodontology 2000 (2010) 53: 111-23]. This allows macrophages that have arrived at the area of the lesion to produce prostaglandin 2 [PGE2]; high levels of this molecule and IL-1β increase the binding of PMNs and monocytes to endothelial cells, exacerbating inflammation, which, together with IL-6 and TNF-α, induce osteoclasts to activate and reabsorb the alveolar bone [Id., citing Dosseva-Panova, V T et al. Subgingival microbial profile and production of proinflammatory cytokines in chronic periodontitis. Folia Med. (2014) 56: 152-60; Meyle, J. and Chapple, I. Molecular aspects of the pathogenesis of periodontitis. Periodontology 2000 (2015) 69: 7-17]. Local capillaries release a large amount of serum as a result of the release of histamine and complement molecules, which leads to increased vascular permeability. This serum is converted into a tissue fluid that contains inflammatory peptides (antibodies, complement, and other agents that mediate the body's defenses) that are carried into the gingival sulcus. Increased gingival fluid causes the tissues and the amount of gingival crevicular fluid to increase in volume [Id., citing Meyle, J. and Chapple, I. Molecular aspects of the pathogenesis of periodontitis. Periodontology 2000 (2015) 69: 7-17]. Macrophages and neutrophils in the infection area contain enzymes (e.g., nicotinamide adenine dinucleotide phosphate (NADPH) oxidase and myeloperoxidase) that produce reactive oxygen species (ROS) to eliminate pathogens [Id., citing Nazam, N. et al. Serum and salivary matrix metalloproteinases, neutrophil elastase, myeloperoxidase in patients with chronic or aggressive periodontitis. Inflammation (2014) 37: 1771-78; Syndergaard, B. et al. Salivary biomarkers associated with gingivitis and response to therapy. J. Periodontol. (2014) 85: e295-e303]. Under normal conditions, antioxidant mechanisms protect the tissues from damage mediated by ROS. However, if the body's antioxidant capacity is insufficient against ROS, oxidative stress occurs that damages the hard and soft tissues of the periodontium [Id., citing Sorsa, T. et al. Matrix metalloproteinases: Contribution to pathogenesis, diagnosis and treatment of periodontal inflammation. Ann. Med. (2006) 38: 306-21; Greabu, M. et al. Hydrogen sulfide, oxidative stress and periodontal diseases: a concise review. Antioxidants (2016) 5: 3]. In addition, excessive release of pro-inflammatory cytokines is stimulated through activation of nuclear factor κB (NFκB) and the production of PGE2, which is related to bone resorption [Id., citing Chapple, I L C and Matthews, JP. The role of reactive oxygen and antioxidant species in periodontal tissue destruction. Periodontology 2000 (2007) 43: 160-232]. If this situation is sustained, the epithelial adhesion is destroyed and the alveolar crest, which is an extension of both the mandible and the maxilla and holds the tooth sockets, loses its height, which translates clinically into dental mobility and formation of periodontal pockets, causing the accumulation of more bacteria that increase the problem, thereby completely destroying the periodontal ligament connecting the cementum of the teeth to the gingivae and alveolar bone; the alveolar bone becomes atrophied, and the tooth is lost. [Id., citing Pihlstrom, B L et al. Periodontal diseases. Lancet (2005) 366: 1809-1820; Corlan Puscu, D. et al. Periodontal disease in diabetic patients-clinical and histopathological aspects. Rom. J. Morphol. Embryol. (2016) 57: 1323-29].

To avoid this outcome, conventional treatment for periodontitis patients is divided into three phases, which often overlap. The first phase is focused on stopping the progression of destruction of periodontal tissues by eliminating local factors through oral hygiene instructions combined with scaling and root planning. The second phase is corrective and is aimed at restoring the function and aesthetics of tissues. The third phase is considered periodontal maintenance which is intended to prevent recurrence of periodontitis. [Id., citing Matos-Cruz, R. et al. Avances (2011) 23: 155-70]. Even when this treatment is carried out with rigor, the results mostly are aimed at the stabilization of the disease and not the regeneration of the lost periodontal tissues. [Id., citing Chen, F M et al. A review on endogenous regenerative technology in periodontal regenerative medicine. Biomaterials (2010) 31: 789207927], other procedures are necessary to recover tissue insertion, including root surface conditioning, bone grafting, guided tissue regeneration, and the application of growth factors. [Id., citing Matos-Cruz, R. et al. Avances (2011) 23: 155-70].

Despite these treatments, the original anatomy and physiology has not been restored, and in some cases, periodontal aberrations, such as ankyloses, gingival recession, and formation of compact bone have developed [Id., citing Oortgiesen, D A et al. Periodontal regeneration using an injectable bone cement combined with BMP-2 or FGF-2. J. Tissue Eng. Regen. Med. (2014) 8: 202-9; Kato, A. et al. Combination of root surface modification with BMP-2 and collagen hydrogel scaffold implantation for periodontal healing in beagle dogs. Open Dent. J. (2015) 9: 52-59].

Biology of Wound Healing

A wound results from damage or disruption to normal anatomical structure and function [Robson M C et al., Curr Probl Surg 2001; 38: 72-140; Velnar T et al., The Journal of International Medical Research 2009; 37: 1528-1542). This can range from a simple break in the epithelial integrity of the skin to deeper, subcutaneous tissue with damage to other structures such as tendons, muscles, vessels, nerves, parenchymal organs and even bone [Alonso J E et al., Surg Clin North Am 1996; 76: 879-903). Irrespective of the cause and form, wounding damages and disrupts the local tissue environment.

Wound healing is a dynamic, interactive process involving soluble mediators, blood cells, extracellular matrix, and parenchymal cells. The wound repair process can be divided into four (4) temporally and spatially overlapping phases: (1) a coagulation phase; (2) an inflammatory phase, (3) a proliferative phase, and (4) a remodeling phase. Much of what is known is based on wound healing of human skin.

Coagulation Phase

Immediately after injury, platelets adhere to damaged blood vessels, initiate a release reaction, and begin a hemostatic reaction, giving rise to a blood-clotting cascade that prevents excessive bleeding and provides provisional protection for the wounded area. Blood platelets release well over a dozen growth factors, cytokines, and other survival or apoptosis-inducing agents [Weyrich A S and Zimmerman G A, Trends Immunol 2004 September; 25(9): 489-495). Key components of the platelet release reaction include platelet-derived growth factor (PDGF) and transforming growth factors A1 and 2 (TGF-A1 and TGF-2), which attract inflammatory cells, such as leukocytes, neutrophils, and macrophages [Singer A F and Clark R A, N Engl J Med 1999 Sep. 2; 341(10): 738-746).

Inflammatory Phase

The inflammatory phase is triggered by capillary damage, which leads to the formation of a blood clot/provisional matrix composed of fibrin and fibronectin. This provisional matrix fills the tissue defect and enables effector cell influx. Platelets present in the clot release multiple cytokines that participate in the recruitment of inflammatory cells (such as neutrophils, monocytes, and macrophages, amongst others), fibroblasts, and endothelial cells (ECs).

Proliferative Phase

The inflammatory phase is followed by a proliferative phase, in which active angiogenesis creates new capillaries, allowing nutrient delivery to the wound site, notably to support fibroblast proliferation. Fibroblasts present in granulation tissue are activated and acquire a smooth muscle cell-like phenotype, then being referred to as myofibroblasts. Myofibroblasts synthesize and deposit extracellular matrix (ECM) components that replace the provisional matrix. They also have contractile properties mediated by α-smooth muscle actin organized in microfilament bundles or stress fibers. Myofibroblastic differentiation of fibroblastic cells begins with the appearance of the protomyofibroblast, whose stress fibers contain only β- and γ-cytoplasmic actins. Protomyofibroblasts can evolve into differentiated myofibroblasts whose stress fibers contain α-smooth muscle actin.

Remodeling Phase

The fourth healing phase involves gradual remodeling of the granulation tissue and reepithelialization. This remodeling process is mediated largely by proteolytic enzymes, especially matrix metalloproteinases (MMPs) and their inhibitors (TIMPs, tissue inhibitors of metalloproteinases). During the reepithelialization, Type III collagen, the main component of granulation tissue, is replaced gradually by type I collagen, the main structural component of the dermis. Elastin, which contributes to skin elasticity and is absent from granulation tissue, also reappears. Cell density normalizes through apoptosis of vascular cells and myofibroblasts (resolution).

Inflammation

Tissue injury causes the disruption of blood vessels and extravasation of blood constituents. The blood clot re-establishes hemostasis and provides a provisional extracellular matrix for cell migration. Platelets not only facilitate the formation of a hemostatic plug but also secrete several mediators of wound healing, such as platelet-derived growth factor, which attract and activate macrophages and fibroblasts [Heldin, C. and Westermark B., In: Clark R., ed. The molecular and cellular biology of wound repair, 2nd Ed. New York, Plenum Press, (1996), at pp. 249-273). It was suggested, however, that, in the absence of hemorrhage, platelets are not essential to wound healing; numerous vasoactive mediators and chemotactic factors are generated by the coagulation and activated-complement pathways and by injured or activated parenchymal cells that were shown to recruit inflammatory leukocytes to the site of injury [Id.].

Ingress of cells into a wound and activation of local cells are initiated by mediators that are either released de novo by resident cells or from reserves stored in the granules of platelets and basophils. [Sephel, G. C. and Woodward, S. C., 3. Repair, Regeneration and Fibrosis,” in Rubin's Pathology, Rubin, R. and Strayer, D. S. Eds; 5^{th} Ed., Wolters Kluwyer Health, /Lippincott Williams & Wilkins, Philadelphia, Pa. (2008), at 71]. Cell migration uses the response of cells to cytokines and insoluble substrates of the extracellular matrix. [Id. at 72].

Infiltrating neutrophils cleanse the wounded area of foreign particles and bacteria and then are extruded with the eschar (a dead tissue that falls off (sheds) from healthy skin or is phagocytosed by macrophages). In response to specific chemoattractants, such as fragments of extracellular-matrix protein, transforming growth factor β (TGF-β), and monocyte chemoattractant protein-1 (MCP-1), monocytes also infiltrate the wound site and become activated macrophages that release growth factors (such as platelet-derived growth factor and vascular endothelial growth factor), which initiate the formation of granulation tissue. Macrophages bind to specific proteins of the extracellular matrix by their integrin receptors, an action that stimulates phagocytosis of microorganisms and fragments of extracellular matrix by the macrophages [Brown, E. Phagocytosis, Bioessays (1995) 17:109-117)( ). Studies have reported that adherence to the extracellular matrix also stimulates monocytes to undergo metamorphosis into inflammatory or reparative macrophages. These macrophages play an important role in the transition between inflammation and repair [Riches, D., In Clark R., Ed. The molecular and cellular biology of wound repair, 2nd Ed. New York, Plenum Press, pp. 95-14]. For example, adherence induces monocytes and macrophages to express Colony-Stimulating Factor-1 (CSF-1), a cytokine necessary for the survival of monocytes and macrophages; Tumor Necrosis Factor-α (TNF-α), a potent inflammatory cytokine; and Platelet-Derived Growth Factor (PDGF), a potent chemoattractant and mitogen for fibroblasts. Other cytokines shown to be expressed by monocytes and macrophages include Transforming Growth Factor (TGF-α), Interleukin-1 (IL-1), Transforming Growth Factor β (TGF-β), and Insulin-like Growth Factor-I (IGF-I) (Rappolee, D. et al., Science, 241, pp. 708-712 (1988)). The monocyte- and macrophage-derived growth factors have been suggested to be necessary for the initiation and propagation of new tissue formation in wounds, because macrophage depleted animals have defective wound repair [Leibovich, S, and Ross, R., Am J Pathol (1975) 78, pp 1-100].

Epithelialization

Reepithelialization of wounds begins within hours after injury. Epidermal cells from skin appendages, such as hair follicles, quickly remove clotted blood and damaged stroma from the wound space. At the same time, the cells undergo phenotypic alteration that includes retraction of intracellular tonofilaments [Paladini, R. et al., J. Cell Biol (1996), 132, pp. 381-397; dissolution of most inter-cellular desmosomes, which provide physical connections between the cells; and formation of peripheral cytoplasmic actin filaments, which allow cell movement and migration [Goliger, J. and Paul, D. Mol Biol Cell, (1995) 6, pp. 1491-1501; Gabbiani, G. et al., J Cell Biol (1978) 76: 561-568]. Furthermore, epidermal and dermal cells no longer adhere to one another, because of the dissolution of hemidesmosomal links between the epidermis and the basement membrane, which allows the lateral movement of epidermal cells. The expression of integrin receptors on epidermal cells allows them to interact with a variety of extracellular-matrix proteins (e.g., fibronectin and vitronectin) that are interspersed with stromal type I collagen at the margin of the wound and interwoven with the fibrin clot in the wound space [Clark, R., J Invest Dermatol. (1990), 94, Suppl: 128S-134S)]. The migrating epidermal cells dissect the wound, separating desiccated eschar (a dead tissue that falls off (sheds) from healthy skin) from viable tissue. The path of dissection appears to be determined by the array of integrins that the migrating epidermal cells express on their cell membranes.

The degradation of the extracellular matrix, which is required if the epidermal cells are to migrate between the collagenous dermis and the fibrin eschar, depends on the production of collagenase by epidermal cells [Pilcher, B. et al., J Cell Biol (1997), 137: 1445-1457], as well as the activation of plasmin by plasminogen activator produced by the epidermal cells [Bugge, T. et al., Cell (1996) 87, 709-719]. Plasminogen activator also activates collagenase (matrix metalloproteinase-1) [Mignatti, P. et al., Proteinases and Tissue Remodeling. In Clark, R. Ed. The molecular and cellular biology of wound repair. 2nd Ed. New York, Plenum Press, (1996) at 427-474] and facilitates the degradation of collagen and extracellular-matrix proteins.

One to two days after injury, epidermal cells at the wound margin begin to proliferate behind the actively migrating cells. The stimuli for the migration and proliferation of epidermal cells during reepithelialization have not been determined, but several possibilities have been suggested. The absence of neighbor cells at the margin of the wound (the “free edge” effect) may signal both migration and proliferation of epidermal cells. Local release of growth factors and increased expression of growth-factor receptors may also stimulate these processes. Leading contenders include Epidermal Growth Factor (EGF), Transforming Growth Factor-α (TGF-α), and Keratinocyte Growth Factor (KGF) [Nanney, L. and King, L. Epidermal Growth Factor and Transforming Growth Factor-α. In Clark, R. Ed. The molecular and cellular biology of wound repair. 2nd Ed. New York, Plenum Press, (1996) pp. 171-194; Werner, S. et al., Science (1994) 266: 819-822; Abraham, J. and Klagsburn, M. Modulation of Wound Repair by Members of the Fibroblast Growth Factor family. In Clark, R. Ed. The molecular and cellular biology of wound repair. 2nd Ed. New York, Plenum Press, (1996) at 195-248]. As re-epithelialization ensues, basement-membrane proteins reappear in a very ordered sequence from the margin of the wound inward, in a zipper-like fashion [Clark R. et al., J. Invest Dermatol. (1982), 79: 264-269]. Epidermal cells revert to their normal phenotype, once again firmly attaching to the reestablished basement membrane and underlying dermis.

Formation of Granulation Tissue

New stroma, often called granulation tissue, begins to invade the wound space approximately four days after injury. Numerous new capillaries endow the new stroma with its granular appearance. Macrophages, fibroblasts, and blood vessels move into the wound space at the same time [Hunt, T. ed. Wound Healing and Wound Infection: Theory and Surgical Practice. New York, Appleton-Century-Crofts (1980)]. The macrophages provide a continuing source of growth factors necessary to stimulate fibroplasia and angiogenesis; the fibroblasts produce the new extracellular matrix necessary to support cell ingrowth; and blood vessels carry oxygen and nutrients necessary to sustain cell metabolism.

Growth factors, especially Platelet-Derived Growth Factor-4 (PDGF-4) and Transforming Growth Factor β-1 (TGF-β1) [Roberts, A. and Sporn, M, Transforming Growth Factor-1, In Clark, R. ed. The molecular and cellular biology of wound repair. 2nd Ed. New York, Plenum Press, (1996) pp. 275-308] in concert with the extracellular-matrix molecules [Gray, A. et al., J Cell Sci. (1993), 104: 409-413; Xu, J. and Clark, R., J Cell Biol. (1996), 132: 239-149], were shown to stimulate fibroblasts of the tissue around the wound to proliferate, express appropriate integrin receptors, and migrate into the wound space. It was reported that platelet-derived growth factor accelerates the healing of chronic pressure sores [Robson, M. et al., Lancet (1992) 339: 23-25] and diabetic ulcers [Steed, D., J Vasc Surg. (1995) 21: 71-78]. In some other cases, basic Fibroblast Growth Factor (bFGF) was effective for treating chronic pressure sores [Robson, M. et al., Ann Surg. (1992) 216: 401-406).

The structural molecules of newly formed extracellular matrix, termed the provisional matrix [Clark, R. et al., J. Invest Dermatol (1982) 79, pp. 264-269,], contribute to the formation of granulation tissue by providing a scaffold or conduit for cell migration. These molecules include fibrin, fibronectin, and hyaluronic acid [Greiling, D. and Clark R., J. Cell Sci (1997), 110: 861-870]. The appearance of fibronectin and the appropriate integrin receptors that bind fibronectin, fibrin, or both on fibroblasts was suggested to be the rate-limiting step in the formation of granulation tissue. While the fibroblasts are responsible for the synthesis, deposition, and remodeling of the extracellular matrix, the extracellular matrix itself can have a positive or negative effect on the ability of fibroblasts to perform these tasks, and to generally interact with their environment [Xu, J. and Clark, R., J Cell Sci (1996) 132: 239-249; Clark, R. et al., J Cell Sci, 108, pp. 1251-1261].

Cell movement into a blood clot of cross-linked fibrin or into tightly woven extracellular matrix requires an active proteolytic system that can cleave a path for cell migration. A variety of fibroblast-derived enzymes, in addition to serum-derived plasmin, are suggested to be potential candidates for this task, including plasminogen activator, collagenases, gelatinase A, and stromelysin [Mignatti, P. et al., Proteinases and Tissue Remodeling. In Clark, R. Ed. The molecular and cellular biology of wound repair. 2nd Ed. New York, Plenum Press, (1996) 427-474; Vaalamo, M. et al., J Invest Dermatol (1997) 109: 96-101]. After migrating into wounds, fibroblasts commence the synthesis of extracellular matrix. The provisional extracellular matrix is replaced gradually with a collagenous matrix, perhaps in response to Transforming Growth Factor-β1 (TGF-β1) signaling [Clark, R. et al., J Cell Sci (1995) 108: 1251-1261; Welch, M. et al., J. Cell Biol (1990) 110:133-145].

Once an abundant collagen matrix has been deposited in the wound, the fibroblasts stop producing collagen, and the fibroblast-rich granulation tissue is replaced by a relatively acellular scar. Cells in the wound undergo apoptosis triggered by unknown signals. It was reported that dysregulation of these processes occurs in fibrotic disorders, such as keloid formation, hypertrophic scars, morphea, and scleroderma.

Neovascularization

The formation of new blood vessels (neovascularization) is necessary to sustain the newly formed granulation tissue. Angiogenesis is a complex process that relies on extracellular matrix in the wound bed as well as migration and mitogenic stimulation of endothelial cells [Madri, J. et al., Angiogenesis in Clark, R. Ed. The molecular and cellular biology of wound repair. 2nd Ed. New York, Plenum Press, (1996) pp. 355-371]. The induction of angiogenesis was initially attributed to acidic or basic Fibroblast Growth Factor. Subsequently, many other molecules have also been found to have angiogenic activity, including vascular endothelial growth factor (VEGF), Transforming Growth Factor-0 (TGF-β), angiogenin, angiotropin, angiopoietin-1, and thrombospondin [Folkman, J. and D'Amore, P, Cell (1996), 87, pp. 1153-1155].

Low oxygen tension and elevated lactic acid were suggested also to stimulate angiogenesis. These molecules induce angiogenesis by stimulating the production of basic Fibroblast Growth Factor (FGF) and Vascular Endothelial Growth Factor (VEGF) by macrophages and endothelial cells. For example, it was reported that activated epidermal cells of the wound secrete large quantities of Vascular Endothelial cell Growth Factor (VEGF) [Brown, L. et al., J Exp Med (1992) 176: 1375-1379)].

Basic fibroblast growth factor was hypothesized to set the stage for angiogenesis during the first three days of wound repair, whereas vascular endothelial-cell growth factor is critical for angiogenesis during the formation of granulation tissue on days 4 through 7 [Nissen, N. et al., Am J Pathol (1998) 152: 1445-1552].

In addition to angiogenesis factors, it was shown that appropriate extracellular matrix and endothelial receptors for the provisional matrix are necessary for angiogenesis. Proliferating microvascular endothelial cells adjacent to and within wounds transiently deposit increased amounts of fibronectin within the vessel wall [Clark, R. et al., J. Exp Med (1982) 156: 646-651). Since angiogenesis requires the expression of functional fibronectin receptors by endothelial cells [Brooks, P. et al., Science (1994) 264: 569-571], it was suggested that perivascular fibronectin acts as a conduit for the movement of endothelial cells into the wound. In addition, protease expression and activity were shown to also be necessary for angiogenesis [Pintucci, G. et al., Semin Thromb Hemost (1996) 22: 517-524].

The series of events leading to angiogenesis has been proposed as follows. Injury causes destruction of tissue and hypoxia. Angiogenesis factors, such as acidic and basic Fibroblast Growth Factor (FGF), are released immediately from macrophages after cell disruption, and the production of vascular endothelial-cell growth factor by epidermal cells is stimulated by hypoxia. Proteolytic enzymes released into the connective tissue degrade extracellular-matrix proteins. Fragments of these proteins recruit peripheral-blood monocytes to the site of injury, where they become activated macrophages and release angiogenesis factors. Certain macrophage angiogenesis factors, such as basic fibroblast growth factor (bFGF), stimulate endothelial cells to release plasminogen activator and procollagenase. Plasminogen activator converts plasminogen to plasmin and procollagenase to active collagenase, and in concert these two proteases digest basement membranes. The fragmentation of the basement membrane allows endothelial cells stimulated by angiogenesis factors to migrate and form new blood vessels at the injured site. Once the wound is filled with new granulation tissue, angiogenesis ceases and many of the new blood vessels disintegrate as a result of apoptosis [Ilan, N. et al., J Cell Sci (1998) 111: 3621-3631]. This programmed cell death has been suggested to be regulated by a variety of matrix molecules, such as thrombospondins 1 and 2, and anti-angiogenesis factors, such as angiostatin, endostatin, and angiopoietin 2 [Folkman, J., Angiogenesis and angiogenesis inhibition: an overview, EXS (1997) 79: 1-8].

Wound Contraction and Extracellular Matrix Reorganization

Wound contraction involves a complex and orchestrated interaction of cells, extracellular matrix, and cytokines. During the second week of healing, fibroblasts assume a myofibroblast phenotype characterized by large bundles of actin-containing microfilaments disposed along the cytoplasmic face of the plasma membrane of the cells and by cell-cell and cell-matrix linkages [Welch, M. et al., J Cell Biol (1990) 110: 133-145; Desmouliere, A. and Gabbiani, G. The role of the myofibroblast in wound healing and fibrocontractive diseases. In Clark, R. Ed. The molecular and cellular biology of wound repair. 2nd Ed. New York, Plenum Press, (1996) pp. 391-423]. The appearance of the myofibroblasts corresponds to the commencement of connective-tissue compaction and the contraction of the wound. This contraction was suggested to require stimulation by Transforming Growth Factor (TGF)-β1 or β2 and Platelet-Derived Growth Factor (PDGF), attachment of fibroblasts to the collagen matrix through integrin receptors, and cross-links between individual bundles of collagen. [Montesano, R. and Orci, Proc Natl Acad Sci USA (1988) 85: 4894-4897; Clark, R. et al., J Clin Invest (1989) 84: 1036-1040; Schiro, J. et al., Cell (1991) 67: 403-410; Woodley, D. et al., J Invest Dermatol. (1991) 97: 580-585].

Collagen remodeling during the transition from granulation tissue to scar is dependent on continued synthesis and catabolism of collagen at a low rate. The degradation of collagen in the wound is controlled by several proteolytic enzymes, termed matrix metalloproteinases (MMP), which are secreted by macrophages, epidermal cells, and endothelial cells, as well as fibroblasts [Mignatti, P. et al., Proteinases and Tissue Remodeling. In Clark, R. Ed. The molecular and cellular biology of wound repair. 2nd Ed. New York, Plenum Press, (1996)427-474]. Various phases of wound repair have been suggested to rely on distinct combinations of matrix metalloproteinases and tissue inhibitors of metalloproteinases [Madlener, M. et al, Exp Cell Res (1998), 242, 201-210].

Wounds gain only about 20 percent of their final strength in the first three weeks, during which fibrillar collagen has accumulated relatively rapidly and has been remodeled by contraction of the wound. Thereafter, the rate at which wounds gain tensile strength is slow, reflecting a much slower rate of accumulation of collagen and collagen remodeling with the formation of larger collagen bundles and an increase in the number of intermolecular cross-links.

Tissue Engineering Considerations

Tissue engineering combines the principles of materials and cell transplantation to develop substitute tissues and/or promote endogenous tissue regeneration. [Furth, M E and Atala, A. Tissue Engineering: Future Perspectives, Chapter 6 In Lanza, R., Langer, R., Vacanti, J. Principles of Tissue Engineering, 4^{th} Ed. Elsevier, Inc. (2014), pp. 83-123]. To support the recovery of tissue function, suitable artificial biomaterials implanted into a wound may act as a matrix for cell growth and tissue formation. [Zhang, R. et al. Hybridization of a phospholipid polymer hydrogel with a natural extracellular matrix using active cell immobilization. Biomaterials Sci. (2019) 7: 2793].

A scaffold used for tissue engineering can be considered an artificial extracellular matrix [Furth, M E and Atala, A. Tissue Engineering: Future Perspectives, Chapter 6 In Lanza, R., Langer, R., Vacanti, J. Principles of Tissue Engineering, 4th Ed. Elsevier, Inc. (2014), pp. 83-123., citing Rosso, F. et al. Smart materials as scaffolds for tissue engineering. J. Cell Physiol. (2005) 203: 465-70]. The normal biological ECM, in addition to contributing to mechanical integrity, has important signaling and regulatory functions in the development, maintenance, and regeneration of tissues. ECM components, in synergy with soluble signals provided by growth factors and hormones, participate in the tissue-specific control of gene expression through a variety of transduction mechanisms. [Id., citing Blum, J L et al. Regulation of mammary differentiation by the extracellular matrix. Environ. Health Perspect. (1989) 80: 71-83; Jones, P L et al. Regulation of gene expression and cell function by extracellular matrix. Crit. Rev. Eukaryot. Gene Expr. (1993) 3: 137-54; Juliano, R L and Haskill, S. Signal transduction from the extracellular matrix. J. Cell Biol. (1993) 120: 577-85; Reid, L. et al. Regulation of growth and differentiation of epithelial cells by hormones, growth factors, and substrates of extracellular matrix. Ann. NY Acad. Sci. (1981): 372: 354-70]. Furthermore, the ECM is itself a dynamic structure that is actively remodeled by the cells with which it interacts. [Id., citing Behonick, D J and Werb, Z. A bit of give and take: the relationship between the extracellular matrix and the developing chondrocyte. Mechanisms of development (2003) 120: 327-36; Birkedal-Hansen, H. Proteolytic remodeling of extracellular matrix. Curr. Opin. Cell Biol. (1995) 7: 728-35].

Decellularized tissues or organs can serve as sources of biological EM for tissue engineering. The relatively high degree of evolutionary conservation of many ECM components allows the use of xenogeneic materials (often porcine). Various extracellular matrices have been utilized successfully for tissue engineering in animals models, and products incorporated decellularized heart valves, small intestinal submucosa and urinary bladder matrix have received regulatory approval for use in human patients[Id., citing Gilbert, T W et al. Decellularization of tissues and organs. Biomaterials (2006): 27: 3675-83]. Despite many advantages, e.g., preservation of 3D structure, preservation of signaling components, there are also concerns about the use of decellularized materials, including the potential for immunogenicity, the possible presence of infectious agents, variability among preparations, and the inability to completely specify and characterize the bioactive components of the material.

Biomaterials can be produced that are capable of interactive behavior, both responsive to and able to modulate the local environment and cellular activities. A number of groups have explored the production of biomaterials that unite the advantages of synthetic polymers with the biological activities of proteins. For example, materials that undergo large conformational change in response to environmental stimuli, such as small changes in temperature, ionic strength or pH have been described [Id., citing Galaev I Y and Mattiasson, B. ‘Smart’ polymers and what they could do in biotechnology and medicine. Trends Biotechnol. (1999) 17: 335-40; Williams, D. Environmentally smart polymers. Medical Device Technology (2005) 16: 9-10, 13]. Light stimuli can activate crosslinking of polymers. [Id., citing Skardal, A. et al. Photocrosslinkable hyaluronan-gelatin hydrogels for two-step bioprinting. Tissue Eng. (2010) 16: 2675-85]. Ultrasound can be used to trigger nanobubble formation and facilitate DNA uptake [Id., citing Watanabe, Y. et al. Delivery of Na/I supporter gene into skeletal muscle using nanobubbles and ultrasound: visualization of gene expression by PET. J. Nuclear Medicine (2010) 51: 951-8]. The responses of a polymer may include precipitation or gelation, reversible adsorption on a surface collapse of a hydrogel or surface graft, and alternation between hydrophilic and hydrophobic states. [Id., citing Hoffman, A S et al. Really smart bioconjugates of smart polymers and receptor proteins. J. Biomed. Materials Res. (20000 52: 477-86].

Biomaterials for Tissue Engineering

Biological polymers are natural biocompatible materials that comprise a whole or a part of a living structure or biomedical device that performs, augments, or replaces a natural function. In recent years there has been a push to investigate natural materials for tissue engineering, especially those natural polymers that are present in the body. Naturally-occurring biopolymers include, but are not limited to, protein polymers, polysaccharides, and photopolymerizable compounds. Protein polymers have been synthesized from self-assembling protein polymers such as, for example, silk fibroin, elastin, collagen, and combinations thereof. Naturally-occurring polysaccharides include, but are not limited to, chitin and its derivatives, hyaluronic acid, dextran and cellulosics (which generally are not biodegradable without modification), and sucrose acetate isobutyrate (SAIB). Chitin is composed predominantly of 2-acetamido-2-deoxy-D-glucose groups and is found in yeasts, fungi and marine invertebrates (shrimp, crustaceans) where it is a principal component of the exoskeleton. Chitin is not water soluble and the deacetylated chitin, chitosan, only is soluble in acidic solutions (such as, for example, acetic acid). Studies have reported chitin derivatives that are water soluble, very high molecular weight (greater than 2 million daltons), viscoelastic, non-toxic, biocompatible and capable of crosslinking with peroxides, gluteraldehyde, glyoxal and other aldehydes and carbodiamides, to form gels.

Synthetic materials have an advantage over natural materials because they can be produced with a fully defined composition and designed features and structures. Many synthetic materials, mostly polymers, have been investigated to act as templates for cartilage regeneration. These include such FDA-approved polymers as poly(lactic acid) (PLA), poly(glycolic acid) (PGA) and polycaprolactone (PCL) and have been formed into either porous, fibrous or hydrogel scaffolds. Ideally, a scaffold should have the following characteristics: (i) three-dimensional and highly porous with an interconnected pore network for cell growth and flow transport of nutrients and metabolic waste; (ii) biocompatible and bioresorbable with a controllable degradation and resorption rate to match cell/tissue growth in vitro and/or in vivo; (iii) suitable surface chemistry for cell attachment, proliferation, and differentiation and (iv) mechanical properties to match those of the tissues at the site of implantation. Properties of many of these polymers have been reviewed [Hutmacher, D W, Biomaterials (2000) 21(24):2529-43.].

Biosynthetic polymers are materials that combine synthetic components with biopolymers or moieties prepared as mimics of those found in nature [Carlini, A S set al. Macromolecules (2016) 49: 4379-94]. These materials consist of (a) synthetically modified biopolymers, such as functionalized hyaluronic acid derivatives [Id., citing Vasi, A-M, et al. Mater. Sci. Eng. C. (2014) 38: 177-85] or labeled proteins via cell-instruction [Id., citing Ngo, J. et al. Proc. Natl Acad. Sci. USA (2013) 110 (13): 4992-7]. In the prior case concerning biopolymers such as polysaccharides or proteins, where reactive sites (amine, hydroxyl, thiol, carboxylic acid) are conventionally present as multiple copies, site-specific conjugation (graft-to) and subsequent purification are typically difficult. Other categories of biosynthetic polymers that enable more precise control over advanced architectures, functionalization, and subsequently dynamic function are (b) biomolecules conjugated to synthetic polymers produced by various grafting strategies [Id., citing Liu, K. et al., J. Am. Chem. Soc. (2014) 136 (40): 14255-62; Rowland, M. et al. Biomacromolecules (2015) 16 (8): 2436-43] or (c) bioinspired or fully synthetic polymers that act as biopolymer surrogates, which execute similar functions and occasionally exceed the performance of biopolymers. [Id., citing Bapat, A P et al. J. Am. Chem. Soc. (2011) 133 (49): 19832-8]. For example, hyaluronic acid (HA), which is composed of alternating glucuronidic and glucosaminidic bonds and is found in mammalian vitreous humor, synovial fluid, unbiblical cords and rooster combs, from which it is isolated and purified, also can be produced by fermentation processes.

Bioadhesive polymers include bioerodible hydrogels [see Sawhney et al Macromolecules (1993) 26, 581-587]. These include polyesters (polyglycolide, polylactic acid and combinations thereof), polyester polyethylene glycol copolymers, polyamino-derived biopolymers, polyanhydrides, polyorthoesters, polyphosphazenes, sucrose acetate isobutyrate (SAM), and photopolymerizable biopolymers, naturally-occurring biopolymers, protein polymers, collagen, polysaccharides, photopolymerizable compounds, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Hydrogel compositions have been used in the medical industry for a variety of purposes, including fabrication of medical devices.

Growth Factors

Growth factors of potential importance in tissue engineering and methods to deliver them have been reviewed. [Id., citing Vasita, R. and Katti, DS. Growth factor delivery systems for tissue engineering: a materials perspective. Expert Rev. Med. Devices (2006) 3: 29-47]. For optimized tissue formation without risk of hyperplasia growth factors should be presented to cells for a limited period of time and in the correct local environment.

Bioactive signals can be incorporated into scaffold materials by chemical linkage. Integrins, transmembrane receptors that serve as adhesion molecules between cells, and other cells and/or the ECM are key targets for ligands used to modify scaffold surfaces. Numerous studies have confirmed that addition of the integrin-binding motif arginine-glycine-aspartic acid [RGD] first identified in fibronectin enhances the binding of many types of cells to a variety of synthetic scaffolds and surfaces, including metals, polymers, potassium phosphate bone surrogates and hydrogels. [Furth, M E and Atala, A. Tissue Engineering: Future Perspectives, Chapter 6 In Lanza, R., Langer, R., Vacanti, J. Principles of Tissue Engineering, 4^{th} Ed. Elsevier, Inc. (2014), pp. 83-123, citing 1Alsberg, E. et al. Engineering growing tissues. Proc. Natl Acad. Sci. USA (2002) 99: 12025-30; Hersel, U. et al. RGD modified polymers: biomaterials for stimulated cell adhesion and beyond. Biomaterials (2003) 24: 4385-15; Liu, J C et al. Comparative cell response to artificial extracellular matrix proteins containing the RGD andDS5 cell-binding domains. Biomacromolecules (2004) 5: 497-504; Meyer, A. et al. Targeting RGD recognizing integrins: drug development, biomaterial research, tumor imaging and targeting. Current Pharmaceutical Design (2006): 12: 2723-47; Ruoslahti, E. RGD and other r4cognition sequences for integrins. Annu. Rec. Cell Devel. Biol. (1996) 12a; 697-715]. Greater selectivity and potency in cellular binding and enhancement of growth and function can be achieved by taking advantage of additional binding motifs in concert with RGD or independently of that tripeptide [Id., citing Alamann, A. et al. RGD, the Rho′d to cell spreading. Eur. J. Cell Biol. (2006) 85: 249-54; Xiao, T. et al. Structural basis for allostery in integrins and binding to fibrinogen-mimetic therapeutics. Nature (2004) 432: 59-67]

Tissue engineering scaffolds serve as temporary devices to facilitate tissue healing and regeneration processes. In skin wound models, for example, healing can be compromised if the scaffold degradation occurs too quickly, whereas scar tissue occurs when the degradation is too slow. [Luo, Yl et al., Chapter 24, 3D Scaffolds In Lanza, R., Langer, R., Vacanti, J. Principles of Tissue Engineering, 4th Ed. Elsevier, Inc. (2014), pp. 475-96, citing Yannas, IV. Facts and Theories of induced organ regeneration. Adv. Biochem. Eng. Biotechnol. ®2005] 93: 1-38]. Optimal skin synthesis and prevention of scar formation could be achieved when the template was replaced by new tissue in a synchronous way, i.e., the time constant for scaffold degradation and the time constant for new tissue synthesis during wound healing were approximately equal [Id.]. The rate of degradation of scaffolds used for tissue engineering therefore is a crucial parameter affecting successful regeneration [[Furth, M E and Atala, A. Tissue Engineering: Future Perspectives, Chapter 6 In Lanza, R., Langer, R., Vacanti, J. Principles of Tissue Engineering, 4th Ed. Elsevier, Inc. (2014), pp. 83-123., citing Alsberg, E. et al. Regulating bone formation via controlled scaffold degradation. J. Dental Res. (2003) 82: 903-8].

In general, there exist lower and upper limits to the optimal degradation rate, which may vary with cellular or tissue processes, scaffold chemical compositions and scaffold functions. Regulation of the degradation rate can be achieved by varying physical parameters of the scaffold, or by engineering the scaffold to contain target sites for proteolytic degradation [Id., Lee, S H et al. Proteolytically degradable hydrogels with a fluorogenic substrate for studies of cellular proteolytic activity and migration. Biotechnol. Prog. (2005) 21: 1736-41; Rizzi, S C et al. Recombinant protein co-PEG networks as cell-adhesive and proteolytically degradable hydrogel matrices. Part II: biofunctional characteristics. Biomacromolecules (2006) 7: 3019-29].

Efforts to Restore the Periodontium to Date

It is widely accepted that for achieving clinically relevant periodontal regeneration, some scaffold architecture is needed to guide the three dimensional growth of tissue. [Liu, Y. et al. Review: development of clinically relevant scaffolds for vascularized bone tissue engineering. Biotechnol. Adv. (2013) 31: 688-705]. Such a scaffold should support cell growth, preferably contain growth factors needed for induction of cell differentiation, should be able to permit the transportation of oxygen, nutrients and waste products, should be biodegradable, and should be highly porous to support these functions. [Id.]

The use of multiple regenerated tissues to reconstruct the periodontal complex remains a major clinical challenge. Some researchers have layered materials and cells using tissue engineering concepts in an effort to mimic the different tissue layers involved in the periodontium [Xu, X-Y, et al. Stem Cells Translational Med. (2019) 8: 392-403, citing Park, C H et al. Advanced engineering strategies for periodontal complex regeneration. Materials (2016) 9: 57]. For example, vertical stacking of three-layered periodontal ligament stem cells (PDLSCs), woven poly(glycolic acid), and porous β-tricalcium phosphate (β-TCP) in an orderly manner based on this concept and their placement into the three-wall periodontal defects of a canine subject resulted in newly formed bone and cementum interspersed with the aligned collagen fibers [Id., citing Silva, N. et al. Host response mechanisms in periodontal diseases. J. Appl. Oral Sci. (2015) 23: 329-55]. Similarly, cell sheets comprising periodontal ligament stem cells (PDLSCs) and/or jaw bone marrow mesenchymal stem cells (BMMSCs) have been multilayered to regenerate a complex periodontium-like architecture [Id., citing Zhang, H. et al. Composite cell sheet for periodontal regeneration: cross-talk between different types of MSCs in cell sheet facilitates complex periodontal-like tissue regeneration. Stem Cell Res. Ther. (2016) 7: 168]. In a similar approach, a sandwich tissue engineering complex was constructed by adding a layer of mineralized membrane on each side of a collagen membrane; after seeding with gingival fibroblasts, this complex was implanted into periodontal defect areas in dogs, and simultaneous neogenesis of ligamentous and osseous structures was achieved. [Id., citing Wu, M. et al. Mineralization induction of gingival fibroblasts and construction of a sandwich tissue-engineered complex for repairing periodontal defects. Med. Sci. Monit. (2018) 24: 1112-3]. 3D-patterned multiphasic complexes have enabled the reconstruction of periodontal complex architectures for periodontal tissue engineering strategies [see, e.g., Park, C H et al. Biomimetic hybrid scaffolds for engineering human tooth-ligament interfaces. Biomaterials (2010): 31: 5945-52; Vaquette, C. et al. A biphasic scaffold design combined with cell sheet technology of simultaneous regeneration of alveolar bone/periodontal ligament complex. Biomaterials (2012) 33: 5560-73; Costa, P F et al. Advanced tissue engineering scaffold design for regeneration of the complex hierarchical periodontal structure. J. Clin. Periodontol. (2014) 41: 283-94; Park, C H et al. Spatio-temporally controlled microchannels of periodontal mimic scaffolds. J. Dent. Res. (2014) 93: 1304-12; Park, C H et al. Image based, fiber guiding scaffolds: a platform for regenerating tissue interfaces. Tissue Eng. Part C. Methods (2014) 20: 533-42; Resperini, G. et al. 3D printed bioresorbable scaffold for periodontal repair. J. Dent. Res. (2015) 94: 1535-75], but their complexity does not necessarily translate to clinical use.

Current strategies for enhancing self-healing focus on directing resident cells for target trafficking and on coaxing these cells to grow new tissues. [Id., citing Wu, R-X et al. Biomaterials for endogenous regenerative medicine: coaxing stem cell homing and beyond. Appl. Materl Technol. (2017) 2: 1700022]. In vitro results have shown that SDF-1-loaded gelatin sponges could release their cargo for up to 35 days and enhance bone/PDL regeneration [Id., citing Cai, X et al. Periodontal regeneration via chemoattractive constructs. J. Clin. Periodontol. (2018) 45: 851-60]. When dipeptidylpeptidase IV (DPP-IV) inhibitor parathyroid hormone (PTH) is used for SDF-1 protection; PTH/SDF1 cotherapy has been found to increase the migration of reparative cells to rat periodontal defects. [Id. citing Wang, F. et al. PTH/SDF-1 cotherapy induces CD90+CD34− stromal cells migration and promotes tissue regeneration in a rat periodontal defect model. Sci. Rep. (2016) 6: 30403]. A tri-layer scaffold reported by Sowmya et al [Id., citing Soemya, D. et al. Tri-layered nanocomposite hydrogel scaffold for the concurrent regeneration of cementum, periodontal ligament, and alveolar bone. Adv. Healthc. Mater. (2017) 6: 1601251] was created to encourage concurrent regeneration of the three types of periodontal tissues; each layer was specifically designed to contain chitin/poly(lactic-co-glycolic acid) (chitin-PLGA) and/or nanobioactive glass ceramic (nBGC) components. A layer composed of chitin-PLGA-nBGC loaded with recombinant human cementum protein-1 was applied to generate cementum; similar components combined with platelet rich plasma were included to regenerate bone. For PDL regeneration, a chitin-PLGA hydrogel was loaded with recombinant human FGF-2. The assessment of this scaffold in a rabbit periodontal defect model showed that the cell-free construct induced regeneration of the hybrid tissues in the periodontium.

Injectable and absorbable scaffolds also have been developed for bone regeneration applications [Liu, J. et al. Cells (2019) 8: 537, citing Tsai H.-C., et al. Novel microinjector for carrying bone substitutes for bone regeneration in periodontal diseases. J. Formos. Med. Assoc. 2016; 115:45-5; Simon C. G., Jr., et al. Preliminary report on the biocompatibility of a moldable, resorbable, composite bone graft consisting of calcium phosphate cement and poly (lactide-co-glycolide) microspheres. J. Orthopaed. Res. 2002; 20:473-482]. Among them, calcium phosphate cements (CPCs) consisting of calcium phosphate powders mixed with a liquid to form a paste could be injected into the bone defect site to harden in situ to form a scaffold, through a dissolution-precipitation reaction at 37° C. [Id., citing Xu H. H., et al. Calcium phosphate cements for bone engineering and their biological properties. Bone Res. 2017; 5:17056]. Cell seeding onto the porous CPC scaffold yielded a relatively poor seeding efficacy and mediocre cell penetration into the scaffold [Id., citing Villalona G. A., et al. Cell-seeding techniques in vascular tissue engineering. Tissue Eng. Part B Rev. 2010; 16:341-350]. It was not feasible to directly mix the cells with the paste due to the mixing stresses, ionic exchanges, and pH variations during the CPC paste setting were harmful to the cells. Subsequently, a resorbable and injectable alginate-microfibers/microbeads (Alg-MB/MF) delivery system for stem cells was developed, which protected the encapsulated stem cells during the CPC paste blending and injection [Id., citing Wang P., et al. A self-setting ipsmsc-alginate-calcium phosphate paste for bone tissue engineering. Dent. Mater. 2016; 32:252-26] and supported cell health, proliferation and differentiation, with microbeads degrading at 3-4 days and releasing the encapsulated cells [Id., citing Grosfeld E.-C., et al. Long-term biological performance of injectable and degradable calcium phosphate cement. Biomed. Mater. 2016; 12:015009; Song Y., et al. Engineering bone regeneration with novel cell-laden hydrogel microfiber-injectable calcium phosphate scaffold. Mater. Sci. Eng. C. 2017; 75:895-905]. Six types of stem cells, human bone mesenchymal stem cells (hBMSCs), human dental pulp stem cells (hDPSCs), human umbilical cord MSCs (hUCMSCs), MSCs derived from embryonic stem cells (hESC-MSCs), human induced pluripotent stem cell-MSCs derived from bone marrow (BM-hiPSC-MSCs) and from foreskin (FS-hiPSC-MSCs), encapsulated in hydrogel microfibers and microbeads inside an injectable CPC were reported to proliferate and osteodifferentiate well, exhibiting high expressions of osteogenic genes at 7 days. [Id., citing Zhao L., et al. An injectable calcium phosphate-alginate hydrogel-umbilical cord mesenchymal stem cell paste for bone tissue engineering. Biomaterials. 2010; 31:6502-6510; Mitsiadis T. A., et al. Dental pulp stem cells, niches, and notch signaling in tooth injury. Adv. Dent. Res. 2011; 23:275-279; Wang P., et al. Bone tissue engineering via human induced pluripotent, umbilical cord and bone marrow mesenchymal stem cells in rat cranium. Acta Biomater. 2015; 18:236-248]. The hBMSC-encapsulated Alginate-microbead-CPC paste implanted into a bone defect for bone regeneration in rats, showed a potent capability for new bone formation; at 12 weeks, an osseous bridge was formed in the bone defect, having an area fraction for the new bone of 42.1%±7.8%, which was three-fold greater than that of the control group [Id., citing Song Y., Engineering bone regeneration with novel cell-laden hydrogel microfiber-injectable calcium phosphate scaffold. Mater. Sci. Eng. C. 2017; 75:895-905].

A tri-culture system that included hiPSC-MSCs, human umbilical vein endothelial cells (HUVECs) and pericytes has been reported to provide pre-vascularization to a CPC scaffold [Id., citing Zhang C., et al. Novel hiPSC-based tri-culture for pre-vascularization of calcium phosphate scaffold to enhance bone and vessel formation. Mater. Sci. Eng. C. 2017; 79:296-3049]. Vessel-like structures successfully formed in both the co-cultured and tri-cultured groups in vitro. In addition, much higher angiogenic and osteogenic marker expressions, as well as bone matrix mineralization, were obtained. the tri-culture group generated much greater new bone amount (45%, 4.5 folds) as well as new blood vessel density (50%, 2.5 folds) in a cranial bone defect model in rats after 12 weeks, when compared with CPC control. The area fraction of the newly-formed bone and the blood vessel density in the tri-culture constructs were approximately 1.2-fold and 1.7-fold those of the co-culture group, respectively [Id., citing Song Y., et al. Engineering bone regeneration with novel cell-laden hydrogel microfiber-injectable calcium phosphate scaffold. Mater. Sci. Eng. C. 2017; 75:895-905].

Although stable periodontal attachment, including cementum and Sharpey's fibers, should be a guide for reforming the instrumented root surface in periodontal regenerative therapy [Kato, A. et al. The Open Dentistry J. (2015) 9: 52-9], it is difficult to achieve these objectives due to rapid junctional epithelium downgrowth, which prevents the formation of periodontal attachment [Id., citing Listgarten M A. Electron microscopic features of the newly formed epithelial attachment after gingival surgery. J Periodont Res. 1967; 2:46]. Even if epithelial tissue does not invade the root surface after healing, many cases show gingival tissue adaptation without periodontal attachment apparatus [Id., citing Nyman S, et al. Healing following implantation of periodontitis-affected roots into gingival connective tissue. J Clin Periodontol. 1980; 7:394-401]. Therefore, compatibility between the root surface and regenerated periodontal tissue is required for a predictable regenerative procedure.

Biomodification of the root dentin surface plays a major role in periodontal healing. Many investigators have confirmed that agents for dentin demineralization remove the surface smear layer, open dentin tubules and expose organic elements such as the collagen matrix, thus increasing total surface area [Id., citing Lasho D J, et al. A scanning electron microscope study of the effects of various agents on instrumented periodontally involved root surfaces. J Periodontol. 1983; 54:210-20; Blomlof J, et al. Effect of different concentrations of EDTA on smear removal and collagen exposure in periodontitis-affected root surfaces. J Clin Periodontol. 1997; 24:534-7]. Various modifications provide a more biocompatible dentin surface; protein absorption, cell migration and attachment and fiber development [Id., citing Pitaru S, et al. The influence of the morphological and chemical nature of dental surfaces on the migration, attachment, and orientation of human gingival fibroblasts in vitro. J Periodont Res. 1984; 19:408-18; Fardal O, and Lowenberg BF. A quantitative analysis of the migration, attachment, and orientation of human gingival fibroblasts to human dental root surfaces in vitro. J Periodontol. 1990; 61:529-35; Blomlof J, and Lindskog S. Root surface texture and early cell and tissue colonization after different etching modalities. Eur J Oral Sci. (1995)103:17-24; Zaman K U, et al. A study of attached and oriented human periodontal ligament cells to periodontally diseased cementum and dentin after demineralizing with neutral and low pH etching solution. J Periodontol. 2000; 71:1094-9]. Demineralized dentin is a suitable surface for retention of growth and differentiation factors [Id., citing Fardal 0, and Lowenberg BF. A quantitative analysis of the migration, attachment, and orientation of human gingival fibroblasts to human dental root surfaces in vitro. J Periodontol. 1990; 61:529-35, Blomlof J, and Lindskog S. Root surface texture and early cell and tissue colonization after different etching modalities. Eur J Oral Sci. (1995) 103:17-24].

Bone morphogenetic proteins (BMPs), known to be biological differentiation factors, have the ability to transform pluripotent stem cells into osteoprogenitor cells [Id., citing Katagiri T, et al. The non-osteogenic mouse pluripotent cell line, C3H10T 1/2, is induced to differentiate into osteoblastic cells by recombinant human bone morphogenetic protein-2. Biochem Biophys Res Commun. (1990) 172:295-9] and to promote ectopic osteogenesis in the body [Id., citing Gong L., Bisphosphonate incadronate inhibits maturation of ectopic bone induced by recombinant human bone induced by recombinant human bone morphogenetic protein-2. J Bone Miner Metab. (2003) 21:5-11, Kim S E, et al. Enhancement of ectopic bone formation by bone morphogenetic protein-2 delivery using heparin-conjugated PLGA nanoparticles with transplantation of bone marrow-derived mesenchymal stem cells. J Biomed Sci. (2008) 15:771-7]. Zaman et al. reported that BMP-applied dentin stimulated the osteogenic activity of attached human periodontal ligament cells [Id., citing Zaman K U, et al. Effect of recombinant human platelet-derived growth factor-BB and bone morphogenetic protein-2 application to demineralized dentin on early periodontal ligament cell response. J Periodont Res. (1999) 34:244-50]. Miyaji et al. presented an in vivo study in which cementum-like tissue was directly formed on the BMP-applied dentin surface in gingival connective tissue [Id., citing Miyaji H, et al. Hard tissue formation on dentin surfaces applied with recombinant human bone morphogenetic protein-2 in the connective tissue of the palate. J Periodont Res. (2002) 37:204-9, Miyaji H, et al. Dentin resorption and cementum-like tissue formation by bone morphogenetic protein application. J Periodont Res. (2006) 41:311-5]. In addition, root surface modification with BMP markedly prevented epithelial downgrowth in experimental periodontal defects in dogs [Id., citing Miyaji H, et al. Root surface conditioning with bone morphogenetic protein-2 facilitates cementum-like tissue deposition in beagle dogs. J Periodont Res. (2010) 45:658-63].

However, Miyaji et al. also demonstrated that root surface modification with BMP frequently causes severe ankylosis and there is little evidence of periodontal ligament formation [Id., citing Miyaji H, et al. Influence of root dentin surface conditioning with bone morphogenetic protein-2 on periodontal wound healing in beagle dogs. J Oral Tissue Engin. (2011) 8:173-8]. BMPs show high proliferative activity on osteoblasts, but low activity on human periodontal ligament cells [Id., citing Zaman K U, et al. Effect of recombinant human platelet-derived growth factor-BB and bone morphogenetic protein-2 application to demineralized dentin on early periodontal ligament cell response. J Periodont Res. (1999) 34:244-50]. In addition, BMP-2 exhibits up-regulation of alkaline phosphatase activity and mineralization of periodontal ligament cells [Saito Y, et al. A cell line with characteristics of the periodontal ligament fibroblasts is negatively regulated for mineralization and Runx2/Cbfa1/Osf2 activity, part of which can be overcome by bone morphogenetic pro-tein-2. J Cell Sci. 2002; 115:4191-200]. Therefore, released BMPs from the root surface may trigger severe ankylosis.

Collagen hydrogel scaffolds may be useful for supplying periodontal ligament cells. Hydrated polymers, such as hydrogel, are an effective scaffold material consisting of synthetic and/or natural copolymers [Id., citing Park J B. The use of hydrogels in bone-tissue engineering. Med Oral Patol Oral Cir Bucal. (2011) 16:115-8]. Previous reports have revealed that activity of periodontal ligament cells is stimulated by application of Type I collagen [Id., citing Hidaka T, et al. A Study on the behaviors of periodontal ligament cells in a gel embedded collagen culture and their suitability for implant seeding. Jpn J Soc Biomater. (1997) 15:63-70]. Therefore, in vitro and in vivo studies have demonstrated ingrowth of fibroblasts, including periodontal ligament cells and vascular endothelial cells, into hydrated collagen gels [Id., citing Matsui R, et al. Application of collagen hydrogel material onto model delayed closing of full-thickness skin defect wound on guinea-pig. Jpn J Artif Organs. (1997) 26:772-8, Ishikawa K, et al. Preparation of biodegradable hydrogel. Jpn J Artif Organs. (1997) 26:791-7]. In addition, collagen hydrogel scaffolds exhibit high degradability, no toxicity and no chronic inflammatory response [Id., citing Miyaji H, et al. The effects of collagen hydrogel implantation in buccal dehiscence defects in beagles. J Oral Tissue Engin. (2007) 5:87-95, Kosen Y, et al. Application of collagen hydrogel/sponge scaffold facilitates periodontal wound healing in class II furcation defects in beagle dogs. J Periodont Res. (2012) 47:626-34]. In dog periodontal healing, applied collagen hydrogel enhanced the growth of cell-rich connective tissue continuous with the pre-existing periodontal ligament along with the root surface [Id., citing Miyaji H, et al. The effects of collagen hydrogel implantation in buccal dehiscence defects in beagles. J Oral Tissue Engin. (2007) 5:87-95]. The collagen hydrogel scaffold also enhanced the formation of new cementum and periodontal ligament, as well as alveolar bone; ankylosis was not detected [Id., citing Kosen Y, et al. Application of collagen hydrogel/sponge scaffold facilitates periodontal wound healing in class II furcation defects in beagle dogs. J Periodont Res. (2012) 47:626-34, Kato A, et al. Periodontal healing by implantation of collagen hydrogel-sponge composite in one-wall infrabony defects in beagle dogs. J Oral Tissue Engin. (2010) 8:39-46].

The effects of BMP modification in conjunction with collagen hydrogel scaffold implantation on periodontal wound healing in dogs has been examined. [Kato, A. et al. Open Dent. J. (2015) 9: 52-9]. The collagen hydrogel scaffold was composed of a type I collagen sponge and a collagen hydrogel. One-wall infrabony defects (5 mm in depth, 3 mm in width) were surgically created in six beagle dogs. In the BMP/Collagen group (BMP/Col), BMP-2 was applied to the root surface (loading dose; 1 μg/μ1), and the defects were filled with collagen hydrogel scaffold, while in the BMP or Collagen group, BMP-2 coating or scaffold implantation was performed. Histometric parameters were evaluated at 4 weeks after surgery. The results showed that single use of BMP stimulated formation of alveolar bone and ankylosis. In contrast, the BMP/Col group showed frequently enhanced reconstruction of periodontal attachment including cementum-like tissue, periodontal ligament and alveolar bone. The amount of new periodontal ligament in the BMP/Col group was significantly greater when compared to all other groups. In addition, ankylosis was rarely observed in the BMP/Col group.

In another study, BMSCs were transfected with BMP-7, seeded on nano-hydroxyapatite/polyamide (nHA/PA) porous scaffolds, and then placed in vivo using a rabbit mandibular defect model [Liu, J. et al. Cells (2019) 8: 537., citing Li G., et al. Nanomaterials for craniofacial and dental tissue engineering. J. Dent. Res. 2017; 96:725-732; Li J., et al. Enhancement of bone formation by bmp-7 transduced MSCs on biomimetic nano-hydroxyapatite/polyamide composite scaffolds in repair of mandibular defects. J. Biomed. Mater. Res. Part A. 2010; 95:973-981]. The scaffolds having BMP-7-transfected MSCs demonstrated a faster response than MSCs/scaffolds and pure nHA/PA scaffolds.

Cells

Studies in research models and humans have supported the principles that cell-seeded scaffolds generally perform much better than synthetic scaffolds alone. [Furth, M E and Atala, A. Tissue Engineering: Future Perspectives, Chapter 6 In Lanza, R., Langer, R., Vacanti, J. Principles of Tissue Engineering, 4^{th} Ed. Elsevier, Inc. (2014), pp. 83-123., citing Atala, A. Engineering organs. Curr. Opin. Biotechnol. (2009) 20: 575-92]. In one early clinical study, tissue-engineered vascular grafts (TEVG), utilizing autologous bone marrow cells seeded onto biodegradable synthetic conduits or patches, were implanted into 42 pediatric patients with congenital heart defects [Id., citing Matsumura, G. et al. First evidence that bone marrow cells contribute to the construction of tissue-engineered vascular autografts in vivo. Circulation (2003) 108: 1729-34; Shin′oka, T. et al. Midterm clinical result of tissue-engineered vascular autografts seeded with autologous bone marrow cells. J. Thoracic Cardiovasc. Surg. (2005) 129: 1330-8]. Safety data showed no evidence of aneurysms or other adverse events after a mean follow-up of 490 days (maximum 32 months) post-surgery. The grafted engineered vessels remained patent and functional, and the vessels increased in diameter as the patients grew. However, detailed analysis demonstrated that donor cells did not contribute directly to the long-term development of regenerated vascular tissue but appeared to play a trophic role in mobilizing host cells from nearby vessels. The investigators later hypothesized that comparable benefits might be obtained with cell-free vascular grafts that would elute cytokines from sophisticated scaffolds to enhance endogenous regenerative responses. [Id., citing Paterson, J T et al. Tissue-engineered vascular grafts for use in the treatment of congenital heart disease: from the bench to the clinic and back again. Regenerative Med. (2012) 7: 409-19].

After seeding of cells onto scaffolds, a period of growth in vitro often is required prior to implantation. Static cell culture conditions generally have proven sub-optimal for the development of engineered neo-tissues because of limited seeking efficiency and poor transport of nutrients, oxygen and wastes. Bioreactor systems have been designed to facilitate the reproducible production of tissue-engineered constructs under tightly controlled conditions [Id., citing Chen, HC and Hu, YC. Bioreactors for tissue engineering. Biotechnol. Lett (2006 28: 1415-23; Freed, L E et al Advanced tools for tissue engineering: scaffolds, bioreactors and signaling. Tissue Eng. (2006) 12: 3285-305; Hansmann, J. et al. Bioreaxtors in tissue engineering—principles, applications and commercial constraints. J. Biotechnol. (2012); Martin, J. et al. The role of bioreactors in tissue engineering. Trends Biotechnol. (2004) 22: 80-86; Martin Y. and Vermette, P. Bioreactors fortissue mass culture: design characterization, and recent advances. Biomaterials (2005) 26: 7481-503; Porrtner, R. et al. Biotreactor design fortissue engineering. J. Biosci. Bioeng. (2005) 100: 235-45]. Bioreactors also may be used to enhance tissue formation through mechanical stimulation [Id., citing Iwasaki, K. et al. Bioengineered three-layered robust and elastic artery using hemodynamically-equivalent pulsatile bioreactor. Circulation (2008) 118: 552-7; Niklason, L E et al. Functional arteries grown in vitro. Science (1999) 284: 489-93; Barron, V. et al Bioreactors for cardiovascular cell and tissue growth: a review. Ann. Biomed. Eng. (2003) 31: 1017-30; Moon, D G et al. Cyclic mechanical preconditioning improves engineered muscle contraction. Tissue Engineering (2008) 14: 473-82; Yazdani, S K et al. Smooth muscle cell seeding of decellularized scaffolds: the importance of bioreactor preconditioning to development of a more native architecture for tissue-engineered blood vessels. Tissue Engineering (2009) 15: 827-40]. Cells also need to migrate in order to form remodeled tissues.

The need to protect grafts from the recipient's immune system is a fundamental problem [Bradley, J A et al. Stem cell medicine encounters the immune system. Nature Recv. Immunol. (2002) 2: 859-71; Fairchild, P. Interview: Immunogenicity: the elephant in the room for regenerative medicine? Regenerative Med. (2013) 8: 23-6].

Adult Stem Cells

Adult stem cells present in many tissues throughout fetal development and postnatal life are committed to restricted cell lineages, and are not intrinsically tumorigenic [Id., citing 448-51]. Such endogenous adult stem cells are embedded within the ECM component of a given tissue compartment, which, along with support cells, form the cellular niche. Such cellular niches within the ECM scaffold together with the surrounding microenvironment contribute important biochemical and physical signals, including growth factors and transcription factors required to initiate stem cell differentiation into committed precursors cells and subsequent precursor cell maturation to form adult tissue cells with specialized phenotypic and functional characteristics.

The most commonly utilized cells for experiments in tissue engineering are mesenchymal stem cells (MSCs), also known as mesenchymal stromal cells [Furth, M E and Atala, A. Tissue Engineering: Future Perspectives, Chapter 6 In Lanza, R., Langer, R., Vacanti, J. Principles of Tissue Engineering, 4^{th} Ed. Elsevier, Inc. (2014), pp. 83-123, citing Pittenger, M F et al. Multilineage potential of adult human mesenchymal stem cells. Science (1999) 284: 143-7] MSCs are non-blood adult stem cells found in a variety of tissues. They are characterized by their spindle-shape morphologically, by the expression of specific markers on their cell surface, and by their ability, under appropriate conditions, to differentiate along a minimum of three lineages (osteogenic, chondrogenic, and adipogenic).

No single marker that definitely delineates MSCs in vivo has been identified due to a lack of consensus regarding the MSC phenotype, but it generally is considered that MSCs are positive for cell surface markers CD105, CD166, CD90, and CD44, and that MSCs are negative for typical hematopoietic antigens, such as CD45, CD34, and CD14. As for the differentiation potential of MSCs, studies have reported that populations of bone marrow-derived MSCs have the capacity to develop into terminally differentiated mesenchymal phenotypes both in vitro and in vivo, including bone, cartilage, tendon, muscle, adipose tissue, and hematopoietic-supporting stroma. Studies using transgenic and knockout mice and human musculoskeletal disorders have reported that MSC differentiate into multiple lineages during embryonic development and adult homeostasis.

MSCs can be harvested readily from fat tissue obtained by liposuction [Furth, M E and Atala, A. Tissue Engineering: Future Perspectives, Chapter 6 In Lanza, R., Langer, R., Vacanti, J. Principles of Tissue Engineering, 4^{th} Ed. Elsevier, Inc. (2014), pp. 83-123., citing Gimble, J M, et al. Adipose-derived stem cells for regenerative medicine. Cir. Res. (2007) 100: 1249-60; Zuk, P A et al. Human adipose tissue is a source of multipotent stem cells. Mol Biol. Cell (2002) 12: 4279-95]. Several sources of MSCs within the orofacial area have been identified, including periodontal ligament stem cells PL-MSCs), apical papilla derived stem cells (SCAP), dental follicle cells (DFCs), and dental pulp mesenchymal stem cells (DP-MSCs) from both deciduous and permanent teeth. SCAP and DFC are stem cells located only in the developing tooth germ before eruption into the oral cavity; SCAP are at the tips of growing teeth; and DFC are in a connective tissue sac surrounding the enamel organ and dental papilla. DP-MSCs express Stro-1, CD29, CD73, CD90, CD105 and CD166, and are negative for haematopoietic markers such as CD14, CD45, CD34, CD25 and CD28 [Hernandez-Monjaraz, B. et al. Intl J. Mol. Sci. (2018) 19: 944., citing Yang, X et al. Mineralized tissue formation by MPG2-transfected pulp stem cells. J. Dent. Res. (2009) 88: 1020-25].

MSCs secrete a subset of a large array of trophic growth factors, cytokines, and other molecules that collectively endow MSCs with potent immunomodulatory, wound healing and regenerative properties [Id., citing Caplan, AI. Adult mesenchymal stem cells for tissue engineering versus regenerative medicine. J. Cell Physiol. (2007) 213: 341-7; Caplan, AI. Why are MSCs therapeutic? New data: new insight. J. Pathol. (2009) 217: 318-24; Caplan, A I and Dennis, JE. Mesenchymal stem cells as trophic mediators. J. Cell Biochem. (2006) 98: 1076-84; Joyce, N. et al. Mesenchymal stem cells for the treatment of neurodegenerative disease. Regenerative Med. (2010) 5: 933-46]. MSCs are thought to orchestrate wound repair by: (1) structural repair via cellular differentiation; (2) immune-modulation; (3) secretion of growth factors that drive neovascularization and re-epithelialization; and (4) mobilization of resident stem cells. (Balaji, S. et al. Adv. Wound Care (2012) 1(40): 159-65).

Results indicate that MSCs play several simultaneous roles: limiting inflammation through releasing cytokines; aiding healing by expressing growth factors; altering host immune responses by secreting immuno-modulatory proteins; enhancing responses from endogenous repair cells; and serving as mature functional cells in some tissues such as bone (Phinney, D G and Pittenger, MF. Stem Cells (2017) 35: 851-58). While pre-clinical studies in experimental animal models of immune and inflammatory disorders have shown great promise using autologous, allogeneic and even xenogeneic MSCs, clinical studies in human subjects have yielded mixed results (Theofilopoulos A N, et al. Nat Immunol. 2017 Jun. 20; 18(7): 716-724).

In experimental disease models including colitis [Theofilopoulos A N, et al. Nat Immunol. 2017 Jun. 20; 18(7): 716-724, citing Zhang Q, et al. J Immunol 2009; 183: 7787-7798)], radiation proctitis [Id., citing Bessout R, et al. Mucosal Immunol 2014; 7: 656-669)], immune thrombocytopenia [Id., citing Xiao J, et al. Transfusion 2012; 52: 2551-2558] and autoimmune encephalomyelitis [Id., citing Zappia E, et al. Blood 2005; 106: 1755-1761], MSCs reduce T-cell proliferation, suppress the inflammatory infiltrates and cytokines and express anti-inflammatory cytokines [Id.]. Similarly, prominent immunosuppressive effects of MSCs for animal immune disorder models of arthritis [Id., citing Zhou B, et al. Clin Immunol 2011; 141: 328-337; Gonzalez M A, et al. Arthritis Rheum 2009; 60: 1006-1019; Liu Y, et al. Arthritis Res Ther 2010; 12: R210], SLE [Id., citing Sun L, et al. Stem Cells 2009; 27: 1421-1432; Chang J W, et al. Cell Transplant 2011; 20: 245-257; Sun J C, et al. Cancer Biol Ther 2010; 10: 368-375; Gu Z, et al. Lupus 2010; 19: 1502-1514], GvHD [Id., citing Guo J, et al. Eur J Haematol 2011; 87: 235-243)] and multiple sclerosis [Id., citing Oh D Y, et al. J Immunol 2012; 188: 2207-2217; Morando S, et al. Stem Cell Res Ther 2012; 3: 3; Liu X J, et al. Clin Exp Immunol 2009; 158: 37-44) have been well documented.

In certain settings, MSCs can even be immunostimulatory. The mechanisms involved in this process are largely unknown. Zhou et al. showed that when mouse spleen T cells were stimulated with allogeneic mixed lymphocyte reaction (MLR) or anti-CD3/CD28 beads and treated with autologous bone marrow MSC or MSC-conditioned medium, MSCs had both suppressive and stimulatory functions toward T cells [Zhou Y, et al. Cytotherapy. 2013 October; 15(10): 1195-207). This depended on the ratio of MSC to responder T cells, with low numbers of MSC increasing and higher numbers inhibiting T-cell proliferation. Immunostimulatory function was mediated, in part, by soluble factors. MSC immunosuppression of the MLR was indirect and related to inhibition of antigen-presenting cell maturation. Direct effects of MSC-conditioned medium during anti-CD3/CD28 stimulated proliferation were entirely stimulatory and required the presence of the T-cell receptor. MSC supernatant contained both CCL2 and CCLS at high levels, but only CCL2 level correlated with the ability to augment proliferation. An anti-CCL2 antibody blocked this proliferative activity. It was therefore determined that CCL2 plays an important role in the immunostimulatory function of MSC, and that the immunomodulatory role of MSC is determined by a balance between inhibitory and stimulatory factors, suggesting the need for caution when these cells are investigated in clinical protocols.

Additionally, Cui et al. (2016) found that MSCs can acquire immunostimulatory properties in certain contexts. MSCs cultured with natural killer (NK) cells primed the NK cells for increased release of IFN-γ (a cytokine critical for innate and adaptive immunity) in response to IL-12 and IL-18 (interleukins produced by activated antigen-presenting cells). Priming of NK cells by MSCs occurred in a cell-cell contact-independent manner and was impaired by inhibition of the CCR2, the receptor of CCL2, on NK cells [Cui R, et al. Stem Cell Res Ther. 2016; 7: 88). Waterman et al. (2010) have suggested that MSCs may polarize into two distinctly acting phenotypes following specific TLR stimulation, resulting in different immune modulatory effects and distinct secretomes [Bernardo M E, Fibbe W E. Cell Stem Cell. 2013 Oct. 3; 13(4): 392-402, citing Waterman R S, et al. PLoS One. 2010 Apr. 26; 5(4): e10088).

This ability of MSCs to adopt a different phenotype in response to sensing an inflammatory environment is not captured in assays that are commonly used to characterize these cells, but it is crucial for understanding their therapeutic potential in immune-mediated disorders since much of the characterization of these properties has been conducted in vitro, and there are outstanding questions about the degree to which they represent activities that are functionally relevant for endogenous and/or transplanted cells in vivo (Id.).

Paracrine Hypothesis

A ‘paracrine hypothesis’ that the observed therapeutic effects of MSCs are partly mediated by stem cell secretion has gained much attention and is supported by experimental data [Arlan, F. et al. Stem Cell Res. (2013) 10: 301-12, citing Gnecchi et al. Circ. Res., 103 (2008): 1204-1219). For example, it has been shown that MSC conditioned medium (MSC-CM) enhanced cardiomyocyte and/or progenitor survival after hypoxia-induced injury [Id., citing Chimenti et al. Circ. Res., 106 (2010): 971-980; Deuse et al. Circulation, 120 (2009): S247-S254; Gnecchi et al. Circ. Res., 103 (2008): 1204-1219; Matsuura et al. J. Clin. Invest., 119 (2009): 2204-2217; Rogers et al., 2011). Furthermore, MSC-CM induces angiogenesis in infarcted myocardium [Id., citing Chimenti et al. Circ. Res., 106 (2010): 971-980; Deuse et al. Circulation, 120 (2009): S247-S254; Li et al. Am. J. Physiol. Heart Circ. Physiol., 299 (2010): H1772-H1781). In both murine and porcine models of myocardial ischemia/reperfusion (I/R) injury it has been shown that MSC-CM reduces infarct size [Id., citing Timmers et al. Stem Cell Res., 1 (2007): 129-137)].

High performance liquid chromatography (HPLC) and dynamic light scatter (DLS) analyses revealed that MSCs secrete cardioprotective microparticles with a hydrodynamic radius ranging from 50 to 65 nm [Id., citing Chen et al., 2011; Lai et al. J. Mol. Cell. Cardiol. (2010) 48: 1215-1224). The therapeutic efficacy of such MSC-derived extracellular vesicles (EVs) was independent of the tissue source of the MSCs. For example, exosomes from human embryonic stem cell-derived MSCs were similar to those derived from other fetal tissue sources (e.g. limb, kidney). This suggested that secretion of therapeutic EVs may be a general property of all MSCs [Id., citing Lai et al. Stem Cell Res., 4 (2010): 214-222)].

MSC-Derived EVs Comprising Exosomes and Microvesicles

Most cells produce EVs as a consequence of intracellular vesicle sorting, including both microvesicles of >200 nm, and exosomes of 50-200 nm diameter. The microvesicles are shed from the plasma membrane, whereas exosomes originate from early endosomes and, as they mature into late endosomes/multivesicular bodies, acquire increasing numbers of intraluminal vesicles, which are released as exosomes upon fusion of the endosome with the cell surface (Phinney, D G and Pittenger, MF. Stem Cells (2017) 35: 851-58)., citing Lee Y, et al. Hum Mol Genet 2012; 21: R15-134; Tkach M, Thery C. Cell 2016; 164: 1226-1232).

MSC-derived EVs, which include exosomes and microvesicles (MV), are involved in cell-to-cell communication, cell signaling, and altering cell or tissue metabolism at short or long distances in the body, and can influence tissue responses to injury, infection, and disease (Their content includes cytokines and growth factors, signaling lipids, mRNAs, and regulatory miRNAs (Id.). The content of MSC EVs is not static; they are a product of the MSC tissue origin, its activities, and the immediate intercellular neighbors of the MSCs (Id.).

MSCs secrete a plethora of biologically active proteins (Id., citing Tremain N, et al. Stem Cells 2001; 19: 408-418; Phinney D G, et al. Stem Cells 2006; 24: 186-198; Ren J, et al. Cytotherapy 2011; 13: 661-674).

Although MSC-derived EVs recapitulate to a large extent the immensely broad therapeutic effects previously attributed to MSCs, most studies fall short of rigorously validating this hypothesis (Id.) For example, various groups have compared the potency of MSCs versus MSC-derived EVs, and in some cases MSC-conditioned media, in animal models of myocardial infarction (Id., citing Bian S, et al. J Mol Med (Berlin) 2014; 92: 387-397), focal cerebral ischemia (Doeppner T R, et al. Stem Cells Transl Med 2015; 4: 1131-1143), gentamicin-induced kidney injury (Reis L A, et al. PLoS One 2012; 7: e44092), and silicosis (Choi M, et al. Mol Cells 2014; 37: 133-1394). While most studies report that MSC-derived EVs are equally effective as MSCs in sparing tissue and/or promoting functional recovery from injury, this desired outcome is compromised by lack of appropriate controls, comparable dosing, evaluation of the different disease endpoints, variations in frequency and timing of dosage, and absence of dose-dependent effects, thereby making it difficult to draw reliable conclusions about comparable efficacy and potency (Id.)

MSC-derived EVs may function largely via horizontal transfer of mRNAs, miRNAs, and proteins, which then function by a variety of mechanisms to alter the activity of target cells. For example, it has been reported that transfer of IGF-1R mRNA from MSC-derived exosomes to cisplatin-damaged proximal tubular epithelial cells sensitized the epithelial cells to the renal-protective effects of locally produced IGF-1 (Id., citing Tomasoni S, et al. Stem Cells Dev 2013; 22: 772-780). With respect to miRNAs, those contained within MSC-derived EVs have been shown to inhibit tumor growth (Id., citing Katakowski M, et al. Cancer Lett 2013; 335: 201-204; Ono M, et al. Sci Signal 2014; 7: ra63), reduce cardiac fibrosis following myocardial infarction (Feng, Y. et al. PLoS One (2014) 9: e88685), stimulate axonal growth from cortical neurons (Id., citing Zhang Y, et al. Mol Neurobiol (2017) 54(4): 2659-73), promote neurite remodeling and functional recovery after stroke (Id., citing Xin H, et al. Stem Cells 2013; 31: 2737-2746), and stimulate endothelial cell angiogenesis (Id., citing Liang X, et al. J Cell Sci 2016; 129: 2182-2189).

Several studies have validated a direct role for exosome-derived miRNAs in modulating target cell function via use of loss-of-function approaches (Id., citing Wang X, et al. Sci Rep 2015; 5: 13721; Xin H, et al. Stem Cells 2013; 31: 2737-2746). Other studies have shown that EVs secreted by bone marrow-derived MSCs contain cystinosin (CTNS), a cystine efflux channel in the lysosomal membrane, and that coculture of fibroblasts and proximal tubular cells from cystinosis patients with MSC-derived EVs resulted in a dose-dependent decrease in cellular cystine levels (Id., citing Iglessias, D M et al. PLoS One (2012) 7: e42840).

It has been demonstrated that exosomes produced from adipose-derived MSCs (ASCs) contain neprilysin, an enzyme that degrades the amyloid beta (Aβ) peptide, and that coculture of N2a cells engineered to overexpress human Aβ with ASCs significantly reduced the levels of secreted Aβ40 and Aβ42 by exosome-mediated transfer of neprilysin (Id., citing Katsuda T, et al. Sci Rep (2013); 3: 1197). A separate study reported that MSC-derived exosomes suppress human-into-mouse graft-versus-host disease (GvHD) by inhibiting Th1 cell effector function via the release of CD73 containing exosomes, which, when taken up by CD39-expressing CD4+Th1 cells, resulted in enhanced adenosine production and increased Th1 cell apoptosis (Id., citing Amarnath A, et al. Stem Cells (2015) 33: 1200-1212). Together, these studies indicate that dissecting the therapeutic effects of MSC-derived EVs and their mechanism of action in vivo may be equally as challenging as determining that for the parent MSCs (Id.).

Not all MSC-derived EVs are equivalent. For example, it has been reported that exosomes isolated from adipose-derived MSCs contain up to fourfold higher levels of enzymatically active neprilysin, as compared to bone marrow-derived MSCs (Id., citing Katsuda T, et al. Sci Rep (2013) 3: 1197). EVs from marrow and umbilical cord-derived MSCs were shown to inhibit the growth and to induce apoptosis of U87MG glioblastoma cells in vitro whereas those from adipose-derived MSCs promoted cell growth but had no effect on U87MG survival (Id., citing Del Fattore, A. et al. Expert Opin. Biol. Ther. (2015) 15: 495-504). Moreover, it has been shown that exosomes prepared from different tissue-specific MSCs have measurably different effects on neurite outgrowth in primary cortical neurons and dorsal root ganglia explant cultures (Id., citing Lopez-Verrilli et al. Neuroscience 2016; 320: 129-139).

The present disclosure provides a hydrogel composition comprising an interpenetrating polymer network (IPN) containing a biopolymer, a first synthetic polymer and a second synthetic polymer in which a contained community of live human MSCs is embedded. The collagen polymer matrix described (a) allows the embedded cells to remain in place or to migrate over short distances; (b) allows diffusion of small molecules, particularly growth factors produced by the cells or provided as a supplement, and EVs released by the cells to support the recovery of periodontium tissue function after injury.

### SUMMARY OF THE INVENTION

In one aspect, the present disclosure provides a periodontal implant configured into a physical form selected from a film, a fiber, a filament, a sheet, a thread, a cylindrical implant, an asymmetrically-shaped implant, a fibrous mesh, or an injectable gel, including an embedded population of at least 0.5×10*5 live cells. In some embodiments, the implant is fabricated from a hydrogel composition including a water content ranging from, e.g., 40% to 92% (w/w inclusive) sufficient to sustain nutritional transport. In some embodiments, the hydrogel composition includes an interpenetrating polymer network containing a biopolymer and two synthetic polymers, the biopolymer is a collagen; and the synthetic polymers are 2-methacryloyloxyethyl phosphorylcholine (MPC) and poly(ethylene glycol)diacrylate (PEGDA). In some embodiments, the two synthetic polymers are at least partially interlaced on a molecular scale to form a polymer matrix but are not covalently bonded to each other and cannot be separated. In some embodiments, the periodontal implant is highly porous and biodegradable. In some embodiments, the periodontal implant may support cell growth and permit the transportation of oxygen, nutrients and waste products.

In some embodiments, the periodontal implant is configured into the physical form by molding. In some embodiments, the injectable gel is capable of being injected with a needle and/or syringe. In some embodiments, the live cells embedded in the polymer matrix are human mesenchymal stem cells. In some embodiments, the live human mesenchymal stem cells are derived from peripheral blood, from adipose tissue, or from dental tissue including craniofacial bone, dental pulp, PDL, a dental follicle, tooth germ, apical papilla, oral mucosa, gingival tissue and periosteum of a normal healthy subject.

In some embodiments, the live human mesenchymal stem cells embedded in the polymer matrix release one or more cell products into the polymer matrix of the implant. In some embodiments, the cell products are delivered to the periodontium by diffusion. In some embodiments, the cell products include: one or more growth factors, fragments or variants thereof; extracellular vesicles (EVs) including a cargo; or both growth factors, fragments or variants thereof and EVs comprising a cargo.

In some embodiments, the one or more growth factors, fragments or variants thereof, cargo, or both growth factors, fragments or variants thereof and EVs including a cargo include one or more of epidermal growth factor (EGF), fibroblast growth factor (FGF), insulin-like growth factor (IGF), platelet derived growth factor (PDGF), transforming growth factor beta (TGFβ), bone morphogenetic proteins (BMPs), and vascular endothelial growth factor (VEGF). In some embodiments, delivery of the formed periodontal implant including the polymer matrix is by surgical placement of the implant at the gum line of a site affected by periodontitis. In some embodiments, the population of live cells embedded in the polymer matrix may release one or more cell products into the polymer matrix by diffusion, chemical reaction or both. In some embodiments, wound healing by the released cell products may be by a paracrine effect.

In some embodiments, at least one surface of the implant once implanted is in contact communication with a affected site. In some embodiments, the embedded population of cells is within 0.400 mm to 0.700 mm, inclusive, of a surface of the implant that is in contact communication with the affected site. In some embodiments, a surface of the implant, the affected site, or both is/are modified to promote its adhesion at the affected site by application of a peptide to the surface of the implant, the affected site, or both. In some embodiments, the peptide is one of amino acid sequence arginine-glycine-aspartic acid (RGD) derived from an ECM protein, arginine-glutamic acid-aspartic acid-valine (REDV) derived from fibronectin; tyrosine-isoleucine-glycine-serine-arginine (YIGSR) derived from laminin; or isoleucine-lysine-valine-alanine-valine (IKVAV) derived from laminin.

In some embodiments, the hydrogel composition includes at least 1%, at least 2%, at least 3%, at least 4%, or at least 5% by weight of the collagen. In some embodiments, a weight ratio of collagen: PEGDA ranges from about 1:3 to about 1:10, inclusive. In some embodiments, a weight ratio of PEGDA/MPC ranges from 1:0.5 to 0.05:1. In some embodiments, the collagen is a natural collagen, a synthetic collagen, a recombinant collagen, or a collagen mimic. In some embodiments, the fibrous mesh is in the form of a woven or nonwoven material. In some embodiments, the fibrous mesh is in the form of a felt, a gauze, or a sponge. In some embodiments, the hydrogel polymer matrix is supplemented with growth factors or their biologically active fragments or variants, EVs or both.

In one aspect, the present disclosure provides a method for treating a site affected by periodontal disease including delivering locally by implant to an affected site an implant including an embedded population of at least 0.5×10*5 live cells. In some embodiments, the implant is fabricated from a hydrogel composition including a water content ranging from, e.g., 40% to 92% (w/w inclusive) sufficient to sustain nutritional transport. In some embodiments, the hydrogel composition includes an interpenetrating polymer network containing a biopolymer and two synthetic polymers, the biopolymer is a collagen; and the synthetic polymers are 2-methacryloyloxyethyl phosphorylcholine (MPC) and poly(ethylene glycol)diacrylate (PEGDA). In some embodiments, the two synthetic polymers are at least partially interlaced on a molecular scale to form a polymer matrix but are not covalently bonded to each other and cannot be separated. In some embodiments, the periodontal implant is highly porous and biodegradable. In some embodiments, the periodontal implant may support cell growth and permit the transportation of oxygen, nutrients and waste products. In some embodiments, the periodontal implant may effect wound healing of the affected site.

In some embodiments, delivery of the formed periodontal implant including the polymer matrix is by surgical placement of the implant at the gum line of a site affected by periodontitis. In some embodiments, the population of live cells embedded in the polymer matrix may release one or more cell products into the polymer matrix by diffusion, chemical reaction or both. In some embodiments, the cell products are delivered to the periodontium by diffusion.

In some embodiments, the method includes configuring the implant into a physical form selected from a film, a fiber, a filament, a sheet, a thread, a cylindrical implant, an asymmetrically-shaped implant or a fibrous mesh. In some embodiments, the configuring of the implant into the physical form is by molding. In some embodiments, the fibrous mesh is in the form of a woven or nonwoven material. In some embodiments, the fibrous mesh is in the form of a felt, a gauze, or a sponge.

In some embodiments, the method includes contacting at least one surface of the implant once implanted with the affected site; wherein the embedded population of cells is within 0.400 mm to 0.700 mm, inclusive, of a surface of the implant that is in contact communication with the affected site. In some embodiments, the method includes modifying a surface of the implant, the affected site, or both to promote its adhesion at the affected site by applying a peptide to the surface of the implant, the affected site, or both.

In some embodiments, the peptide is one of amino acid sequence arginine-glycine-aspartic acid (RGD) derived from an ECM protein; arginine-glutamic acid-aspartic acid-valine (REDV) derived from fibronectin; tyrosine-isoleucine-glycine-serine-arginine (YIGSR) derived from laminin; or isoleucine-lysine-valine-alanine-valine (IKVAV) derived from laminin. In some embodiments, the live cells embedded in the polymer matrix are human mesenchymal stem cells. In some embodiments, the live human mesenchymal stem cells are derived from peripheral blood, from adipose tissue, or from dental tissue including craniofacial bone, dental pulp, PDL, a dental follicle, tooth germ, apical papilla, oral mucosa, gingival tissue and periosteum of a normal healthy subject. In some embodiments, the live human mesenchymal stem cells embedded in the polymer matrix release one or more cell products into the polymer matrix of the implant.

In some embodiments, the cell products include: one or more growth factors, fragments or variants thereof; extracellular vesicles (EVs) comprising a cargo; or both growth factors, fragments or variants thereof and EVs comprising a cargo. In some embodiments, the one or more growth factors, fragments or variants thereof, or cargo, or both include one or more of epidermal growth factor (EGF), fibroblast growth factor (FGF), insulin-like growth factor (IGF), platelet derived growth factor (PDGF), transforming growth factor beta (TGFβ), bone morphogenetic proteins (BMPs), and vascular endothelial growth factor (VEGF).

In some embodiments, wound healing of the affected site by the released cell products is by a paracrine effect. In some embodiments, the hydrogel composition includes at least 1%, at least 2%, at least 3%, at least 4%, or at least 5% by weight of the collagen. In some embodiments, a weight ratio of collagen: PEGDA ranges from about 1:3 to about 1:10, inclusive. In some embodiments, a weight ratio of PEGDA/MPC ranges from 1:0.5 to 0.05:1. In some embodiments, the collagen is a natural collagen, a synthetic collagen, a recombinant collagen, or a collagen mimic. In some embodiments, the method includes supplementing the hydrogel polymer matrix in situ with growth factors or their biologically active fragments or variants, EVs or both.

### BRIEF DESCRIPTION OF THE DRAWINGS

FIG. 1A is a schematic representation of the periodontium containing the intact bone-PDL-cementum and FIG. 1B is a schematic representation of damage to the periodontium as a result of disease, which leads to loss of multiple periodontal tissues surrounding and supporting the tooth. [Taken from Xu, X-Y, et al. Stem Cell Translational Med. (92019) 8: 392-403, FIG. 2].

FIG. 2 is a schematic diagram illustrating an MTT (3-(4,5-dimethylthiazol-2-yl)-2,5-diphenyltetrazolium bromide) assay;

FIG. 3 is an image of cell coverage on a biocompatible material after seven days;

FIG. 4 is an image of cell coverage on a non-biocompatible material after seven days;

FIG. 5 is a bar graph showing thickness for different samples tested in the cell attachment assay at day 4;

FIG. 6 is a bar graph showing thickness for different samples tested in the cell attachment assay at day 7;

FIG. 7 is a bar graph showing thickness over time for different samples tested in the cell attachment assay;

FIGS. 8A, 8B, 8C, 8D, 8E, 8F, and 8G are microscopy images for different samples tested in the cell attachment assay at day 4, with FIG. 8A showing a control, FIG. 8B showing Nippi 10%, FIG. 8C showing Nippi 12%, FIG. 8D showing Nippi 15%, FIG. 8E showing Nippon 10%, FIG. 8F showing Ferentis 1823B, and FIG. 8G showing Ferentis 1837A;

FIGS. 9A, 9B, 9C, 9D, 9E, 9F, and 9G are microscopy images for different samples tested in the cell attachment assay at day 7, with FIG. 9A showing a control, FIG. 9B showing Nippi 10%, FIG. 9C showing Nippi 12%, FIG. 9D showing Nippi 15%, FIG. 9E showing Nippon 10%, FIG. 9F showing Ferentis 1823B, and FIG. 9G showing Ferentis 1837A;

FIG. 10 is a diagram illustrating placement of materials then seeded with cells during a cell attachment assay;

FIG. 11 is a bar graph showing thickness over time for different samples tested in the cell attachment assay;

FIGS. 12A, 12B, 12C, 12D, 12E, 12F, 12G, 12H, and 12I are microscopy images for different samples tested in the cell attachment assay at day 4, with FIG. 12A showing a control, FIG. 12B showing Ferentis 1842A, FIG. 12C showing Nippi 12% D12%, FIG. 12D showing Nippi 10% D10%, FIG. 12E showing Nippi 12% D10%, FIG. 12F showing Nippon 10%, FIG. 12G showing SA-13-31B, FIG. 1211 showing SA-13-92A edge, and FIG. 121 showing SA-13-92A on sample;

FIGS. 13A, 13B, 13C, 13D, 13E, 13F, 13G, 13H, and 13I are microscopy images for different samples tested in the cell attachment assay at day 7, with FIG. 13A showing a control, FIG. 13B showing Ferentis 1842A, FIG. 13C showing Nippi 12% D12%, FIG. 13D showing Nippi 10% D10%, FIG. 13E showing Nippi 12% D10%, FIG. 13F showing Nippon 10%, FIG. 13G showing SA-13-31B, FIG. 1311 showing SA-13-92A edge, and FIG. 131 showing SA-13-92A on sample;

FIGS. 14A, 14B, 14C, 14D, 14E, and 14F are microscopy images for control samples tested in the cell attachment assay, with each of FIGS. 14A-14F showing control samples and, in particular, FIGS. 14A-14D showing control sample images for 4/6 samples, 80-100% confluent, and FIGS. 14E-14F showing control sample images for 2/6 samples mostly confluent, and a few patches in center;

FIGS. 15A, 15B, 15C, 15D, 15E, 15F, 15G, 15H, 15I, and 15J are microscopy images for 1745A samples tested in the cell attachment assay, with FIGS. 15A-15C showing 1745A sample images for 3/10 confluent at edges and nearly confluent in center, FIGS. 15D-15E showing 1745A sample images for 2/10 60-70% confluent in center, confluent at edges, and FIGS. 15F-15J showing 1745A sample images for 5/10 samples 30-40% confluent in center, patchy, some holes;

FIG. 16 is an image of an MTT plate illustrating the setup for samples tested in the cell attachment assay;

FIG. 17 is a bar graph showing cell numbers for MTT results in the cell attachment assay for a sample and control;

FIGS. 18A, 18B, and 18C are images of collagen implants in the form of scaffolds used to treat gum disease; and

FIG. 19 is an image of an injectable hydrogel scaffold used to treat gum disease.

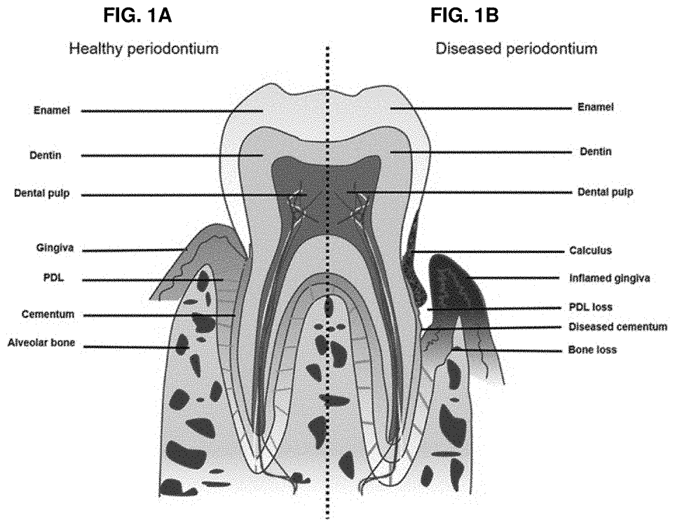

### DETAILED DESCRIPTION OF THE INVENTION

### Glossary

The term “about” or “approximately” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

As used herein and in the appended claims, the singular forms “a”, “an”, and “the” include plural reference unless the context clearly dictates otherwise. Thus, for example, reference to a “polymer” is a reference to one or more polymers and equivalents thereof known to those skilled in the art, and so forth.

The term “adaptive immune response” refers to an immune response mediated by uniquely specific recognition or a non-self entity by lymphocytes whose activation leads to elimination of the entity and the production of specific memory lymphocytes. Because these memory lymphocytes forestall disease in subsequent attacks by the same pathogen, the host immune system is said to have “adapted” to copy with the entity.

The term “adhere” and its other grammatical forms as used herein means to stick fast to a surface or substance.

The term “administer” and its other grammatical forms as used herein means to give or to apply. It includes in vivo administration, as well as administration directly to tissue ex vivo.

The term “admixture” or “blend” is generally used herein to refer to a physical combination of two or more different components. In the case of polymers, an admixture is a physical combination of two or more different polymers.

The term “alveolar bone” as used herein refers to that part of the maxilla and mandible which supports the teeth by forming an attachment for fibers of the periodontal ligament; it consists of two plates of cortical bone separated by spongy bone.

The term “alveolar crest” as used herein refers to the most coronal portion, or the top of the alveolar process or alveolar bone, which is the thick ridge of bone which contains the tooth sockets.

Anatomical Terms

When referring to animals that typically have one end with a head and mouth, with the opposite end often having the anus and tail, the head end is referred to as the cranial end, while the tail end is referred to as the caudal end. Within the head itself, rostral refers to the direction toward the end of the nose, and caudal is used to refer to the tail direction. The surface or side of an animal's body that is normally oriented upwards, away from the pull of gravity, is the dorsal side; the opposite side, typically the one closest to the ground when walking on all legs, swimming or flying, is the ventral side. On the limbs or other appendages, a point closer to the main body is “proximal”; a point farther away is “distal”. Three basic reference planes are used in zoological anatomy. A “sagittal” plane divides the body into left and right portions. The “midsagittal” plane is in the midline, i.e. it would pass through midline structures such as the spine, and all other sagittal planes are parallel to it. A “coronal” plane divides the body into dorsal and ventral portions. A “transverse” plane divides the body into cranial and caudal portions.

When referring to humans, the body and its parts are always described using the assumption that the body is standing upright. Portions of the body which are closer to the head end are “superior” (corresponding to cranial in animals), while those farther away are “inferior” (corresponding to caudal in animals). Objects near the front of the body are referred to as “anterior” (corresponding to ventral in animals); those near the rear of the body are referred to as “posterior” (corresponding to dorsal in animals). A transverse, axial, or horizontal plane is an X-Y plane, parallel to the ground, which separates the superior/head from the inferior/feet. A coronal or frontal plane is an Y-Z plane, perpendicular to the ground, which separates the anterior from the posterior. A sagittal plane is an X-Z plane, perpendicular to the ground and to the coronal plane, which separates left from right. The midsagittal plane is the specific sagittal plane that is exactly in the middle of the body.

Structures near the midline are called medial and those near the sides of animals are called lateral. Therefore, medial structures are closer to the midsagittal plane, lateral structures are further from the midsagittal plane. Structures in the midline of the body are median. For example, the tip of a human subject's nose is in the median line.

The term “ipsilateral” as used herein means on the same side, the term “contralateral” as used herein means on the other side, and the term “bilateral” as used herein means on both sides. Structures that are close to the center of the body are proximal or central, while ones more distant are distal or peripheral. For example, the hands are at the distal end of the arms, while the shoulders are at the proximal ends.

The term “ankyloses” as used herein refers to aberrant healing following periodontal therapy in which regenerated bone binds directly to the instrumented surface of the tooth root.

The term “biocompatible” as used herein, means causing no clinically relevant tissue irritation, injury, toxic reaction, or immunologic reaction to human tissue based on a clinical risk/benefit assessment.

The term “biodegradable” as used herein refers to a material that will erode to soluble species or that will degrade under physiologic conditions to smaller units or chemical species that are, themselves, non-toxic (biocompatible) to the subject and capable of being metabolized, eliminated, or excreted by the subject. The two major types for biodegradation of delivered biodegradable polymeric material are chemical and enzymic degradation, e.g., by hydrolysis and oxidation-dependent degradation Polymers also may degrade by mechanical or thermal processes. [Vhora, I. et al. Applications of Polymers in Drug Delivery, Ch. 8, pages 221-61, at 228; Elsevier, Inc. (2021)

Bone Anatomy

Grossly, two types of bone may be distinguished: cancellous, trabecular or spongy bone, and cortical, compact, or dense bone.

Cortical bone. Cortical bone, also referred to as compact bone or dense bone, is the tissue of the hard outer layer of bones, so-called due to its minimal gaps and spaces. This tissue gives bones their smooth, white, and solid appearance. Cortical bone consists of haversian sites (the canals through which blood vessels and connective tissue pass in bone) and osteons (the basic units of structure of cortical bone comprising a haversian canal and its concentrically arranged lamellae), so that in cortical bone, bone surrounds the blood supply. Cortical bone has a porosity of about 5% to about 30%, and accounts for about 80% of the total bone mass of an adult skeleton.

Cancellous Bone (Trabecular or Spongy Bone). Cancellous bone tissue, an open, cell-porous network also called trabecular or spongy bone, fills the interior of bone and is composed of a network of rod- and plate-like elements that make the overall structure lighter and allows room for blood vessels and marrow so that the blood supply surrounds bone. Cancellous bone accounts for the remaining 20% of total bone mass but has nearly ten times the surface area of cortical bone. It does not contain haversian sites and osteons and has a porosity of about 30% to about 90%.

The head of a bone, termed the epiphysis, has a spongy appearance and consists of slender irregular bone trabeculae, or bars, which anastomose to form a lattice work, the interstices of which contain the marrow, while the thin outer shell appears dense. The irregular marrow spaces of the epiphysis become continuous with the central medullary cavity of the bone shaft, termed the diaphysis, whose wall is formed by a thin plate of cortical bone.

Both cancellous and cortical bone have the same types of cells and intercellular substance, but they differ from each other in the arrangement of their components and in the ratio of marrow space to bone substance. In cancellous bone, the marrow spaces are relatively large and irregularly arranged, and the bone substance is in the form of slender anastomosing trabeculae and pointed spicules. In cortical bone, the spaces or channels are narrow and the bone substance is densely packed.

With very few exceptions, the cortical and cancellous forms are both present in every bone, but the amount and distribution of each type vary considerably. The diaphyses (shafts) of the long bones consist mainly of cortical tissue; only the innermost layer immediately surrounding the medullary cavity is cancellous bone. The tabular bones of the head are composed of two plates of cortical bone enclosing marrow space bridged by irregular bars of cancellous bone. The epiphyses of the long bones and most of the short bones consist of cancellous bone covered by a thin outer shell of cortical bone.

Each bone, except at its articular end, is surrounded by a vascular fibroelastic coat, the periosteum. The so-called endosteum, or inner periosteum of the marrow cavity and marrow spaces, is not a well-demarcated layer; it consists of a variable concentration of medullary reticular connective tissue that contains osteogenic cells that are in immediate contact with the bone tissue.

Components of Bone

Bone is composed of cells and an intercellular matrix of organic and inorganic substances. The organic fraction consists of collagen, glycosaminoglycans, proteoglycans, and glycoproteins. The protein matrix of bone largely is composed of collagen, a family of fibrous proteins that have the ability to form insoluble and rigid fibers. The main collagen in bone is type I collagen. The inorganic component of bone, which is responsible for its rigidity and may constitute up to two-thirds of its fat-free dry weight, is composed chiefly of calcium phosphate and calcium carbonate, in the form of calcium hydroxyapatite, with small amounts of magnesium hydroxide, fluoride, and sulfate. The composition varies with age and with a number of dietary factors. The bone minerals form long fine crystals that add strength and rigidity to the collagen fibers; the process by which it is laid down is termed mineralization.

Bone Cells

Four cell types in bone are involved in its formation and maintenance. These are 1) osteoprogenitor cells, 2) osteoblasts, 3) osteocytes, and 4) osteoclasts.

Osteoprogenitor Cells. Osteoprogenitor cells arise from mesenchymal cells, and occur in the inner portion of the periosteum (defined below) and in the endosteum (defined below) of mature bone. They are found in regions of the embryonic mesenchymal compartment where bone formation is beginning and in areas near the surfaces of growing bones. Structurally, osteoprogenitor cells differ from the mesenchymal cells from which they have arisen. They are irregularly shaped and elongated cells having pale-staining cytoplasm and pale-staining nuclei. Osteoprogenitor cells, which multiply by mitosis, are identified chiefly by their location and by their association with osteoblasts. Some osteoprogenitor cells differentiate into osteocytes. While osteoblasts and osteocytes are no longer mitotic, it has been shown that a population of osteoprogenitor cells persists throughout life.

Osteoblasts. Osteoblasts, which are located on the surface of osteoid seams (meaning the narrow region on the surface of a bone of newly formed organic matrix not yet mineralized), are derived from osteoprogenitor cells. They are immature, mononucleate, bone-forming cells that synthesize collagen and control mineralization. Osteoblasts can be distinguished from osteoprogenitor cells morphologically; generally they are larger than osteoprogenitor cells, and have a more rounded nucleus, a more prominent nucleolus, and cytoplasm that is much more basophilic. Osteoblasts make a protein mixture known as osteoid, primarily composed of type I collagen, which mineralizes to become bone. Osteoblasts also manufacture hormones, such as prostaglandins, alkaline phosphatase, an enzyme that has a role in the mineralization of bone, and matrix proteins.

Osteocytes. Osteocytes, star-shaped mature bone cells derived from ostoblasts and the most abundant cell found in compact bone, maintain the structure of bone. Osteocytes, like osteoblasts, are not capable of mitotic division. They are actively involved in the routine turnover of bony matrix and reside in small spaces, cavities, gaps or depressions in the bone matrix called lacuna. Osteocytes maintain the bone matrix, regulate calcium homeostasis, and are thought to be part of the cellular feedback mechanism that directs bone to form in places where it is most needed. Bone adapts to applied forces by growing stronger in order to withstand them; osteocytes may detect mechanical deformation and mediate bone-formation by osteoblasts.

Osteoclasts. Osteoclasts, which are derived from a monocyte stem cell lineage and possess phagocytic-like mechanisms similar to macrophages, often are found in depressions in the bone referred to as Howship's lacunae. They are large multinucleated cells specialized in bone resorption. During resorption, osteoclasts seal off an area of bone surface; then, when activated, they pump out hydrogen ions to produce a very acid environment, which dissolves the hydroxyapatite component. The number and activity of osteoclasts increase when calcium resorption is stimulated by injection of parathyroid hormone (PTH), while osteoclastic activity is suppressed by injection of calcitonin, a hormone produced by thyroid parafollicular cells.

Bone Matrix. The bone matrix accounts for about 90% of the total weight of compact bone and is composed of microcrystalline calcium phosphate resembling hydroxyapatite (60%) and fibrillar type I collagen (27%). The remaining 3% consists of minor collagen types and other proteins including osteocalcin, osteonectin, osteopontin, bone sialoprotein, as well as proteoglycans, glycosaminoglycans, and lipids.

Bone matrix is also a major source of biological information that skeletal cells can receive and act upon. For example, extracellular matrix glycoproteins and proteoglycans in bone bind a variety of growth factors and cytokines, and serve as a repository of stored signals that act on osteoblasts and osteoclasts. Examples of growth factors and cytokines found in bone matrix include, but are not limited to, Bone Morphogenic Proteins (BMPs), Epidermal Growth Factors (EGFs), Fibroblast Growth Factors (FGFs), Platelet-Derived Growth Factors (PDGFs), Insulin-like Growth Factor-1 (IGF-1), Transforming Growth Factors (TGFs), Bone-Derived Growth Factors (BDGFs), Cartilage-Derived Growth Factor (CDGF), Skeletal Growth Factor (hSGF), Interleukin-1 (IL-1), and macrophage-derived factors.

There is an emerging understanding that extracellular matrix molecules themselves can serve regulatory roles, providing both direct biological effects on cells as well as key spatial and contextual information.

The Periosteum and Endosteum. The periosteum is a fibrous connective tissue investment of bone, except at the bone's articular surface. Its adherence to the bone varies by location and age. In young bone, the periosteum is stripped off easily. In adult bone, it is more firmly adherent, especially at the insertion of tendons and ligaments, where more periosteal fibers penetrate into the bone as the perforating fibers of Sharpey (bundles of collagenous fibers that pass into the outer circumferential lamellae of bone). The periosteum consists of two layers, the outer of which is composed of coarse, fibrous connective tissue containing few cells but numerous blood vessels and nerves. The inner layer, which is less vascular but more cellular, contains many elastic fibers. During growth, an osteogenic layer of primitive connective tissue forms the inner layer of the periosteum. In the adult, this is represented only by a row of scattered, flattened cells closely applied to the bone. The periosteum serves as a supporting bed for the blood vessels and nerves going to the bone and for the anchorage of tendons and ligaments. The osteogenic layer, which is considered a part of the periosteum, is known to furnish osteoblasts for growth and repair, and acts as an important limiting layer controlling and restricting the extend of bone formation. Because both the periosteum and its contained bone are regions of the connective tissue compartment, they are not separated from each other or from other connective tissues by basal laminar material or basement membranes. Perosteal stem cells have been shown to be important in bone regeneration and repair. (Zhang et al., 2005, J. Musculoskelet. Neuronal. Interact. 5(4): 360-362).

The endosteum lines the surface of cavities within a bone (marrow cavity and central canals) and also the surface of trabeculae in the marrow cavity. In growing bone, it consists of a delicate striatum of myelogenous reticular connective tissue, beneath which is a layer of osteoblasts. In the adult, the osteogenic cells become flattened and are indistinguishable as a separate layer. They are capable of transforming into osteogenic cells when there is a stimulus to bone formation, for example, after a fracture.

Bone Marrow. The marrow is a soft connective tissue that occupies the medullary cavity of the long bones, the larger central canals, and all of the spaces between the trabeculae of spongy bone. It consists of a delicate reticular connective tissue, in the meshes of which lie various kinds of cells. Two varieties of marrow are recognized: red and yellow. Red marrow is the only type found in fetal and young bones, but in the adult it is restricted to the vertebrae, sternum, ribs, cranial bones, and epiphyses of long bones. It is the chief site for the genesis of blood cells in the adult body. Yellow marrow consists primarily of fat cells that gradually have replaced the other marrow elements. Under certain conditions, the yellow marrow of old or emaciated persons loses most of its fat and assumes a reddish color and gelatinous consistency, known as gelatinous marrow. With adequate stimulus, yellow marrow may resume the character of red marrow and play an active part in the process of blood development.

Osteogenesis. There are two major modes of bone formation, or osteogenesis, and both involve the transformation of a preexisting mesenchymal tissue into bone tissue. The direct conversion of mesenchymal tissue into bone is called intramembranous ossification. This process occurs primarily in the bones of the skull. In other cases, mesenchymal cells differentiate into cartilage, which is later replaced by bone. The process by which a cartilage intermediate is formed and replaced by bone cells is called endochondral ossification.

Endochondral ossification, which involves the in vivo formation of cartilage tissue from aggregated mesenchymal cells, and the subsequent replacement of cartilage tissue by bone, can be divided into five stages. First, the mesenchymal cells are committed to become cartilage cells. This commitment is caused by paracrine factors that induce the nearby mesodermal cells to express two transcription factors, Paxl and Scleraxis, which are known to activate cartilage-specific genes. During the second phase of endochondral ossification, the committed mesenchyme cells condense into compact nodules and differentiate into chondrocytes (cartilage cells that produce and maintain the cartilaginous matrix, which consists mainly of collagen and proteoglycans). Studies have shown that N-cadherin is important in the initiation of these condensations, and neural cell adhesion molecule (N-CAM) is important for maintaining them. In humans, the SOX9 gene, which encodes a DNA-binding protein, is expressed in the precartilaginous condensations. During the third phase of endochondral ossification, the chondrocytes proliferate rapidly to form the model for bone. As they divide, the chondrocytes secrete a cartilage-specific extracellular matrix. In the fourth phase, the chondrocytes stop dividing and increase their volume dramatically, becoming hypertrophic chondrocytes. These large chondrocytes alter the matrix they produce (by adding collagen X and more fibronectin) to enable it to become mineralized by calcium carbonate. The fifth phase involves the invasion of the cartilage model by blood vessels. The hypertrophic chondrocytes die by apoptosis, and this space becomes bone marrow. As the cartilage cells die, a group of cells that have surrounded the cartilage model differentiate into osteoblasts, which begin forming bone matrix on the partially degraded cartilage. Eventually, all the cartilage is replaced by bone. Thus, the cartilage tissue serves as a model for the bone that follows.

Bone Remodeling. Bone constantly is broken down by osteoclasts and re-formed by osteoblasts in the adult. It has been reported that as much as 18% of bone is recycled each year through the process of renewal, known as bone remodeling, which maintains bone's rigidity. The balance in this dynamic process shifts as people grow older: in youth, it favors the formation of bone, but in old age, it favors resorption.

As new bone material is added peripherally from the internal surface of the periosteum, there is a hollowing out of the internal region to form the bone marrow cavity. This destruction of bone tissue is due to osteoclasts that enter the bone through the blood vessels. Osteoclasts dissolve both the inorganic and the protein portions of the bone matrix. Each osteoclast extends numerous cellular processes into the matrix and pumps out hydrogen ions onto the surrounding material, thereby acidifying and solubilizing it. The blood vessels also import the blood-forming cells that will reside in the marrow for the duration of the organism's life.

The number and activity of osteoclasts must be tightly regulated. If there are too many active osteoclasts, too much bone will be dissolved, and osteoporosis will result. Conversely, if not enough osteoclasts are produced, the bones are not hollowed out for the marrow, and osteopetrosis (known as stone bone disease, a disorder whereby the bones harden and become denser) will result.

The term “collagen” as used herein refers to a natural, chemically synthesized, or synthetic protein rich in glycine and proline that in vivo is a major component of the extracellular matrix and connective tissues.

The term “conditioned medium” (or plural, media), as used herein refers to spent culture medium harvested from cultured cells containing metabolites, growth factors, EVs, RNA and proteins released into the medium by the cultured cells.

The term “contact” and its various grammatical forms as used herein refers to a state or condition of touching or of immediate or local proximity. Contacting a composition to a target destination may occur by any means of administration known to the skilled artisan.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “deliver” and its other grammatical forms as used herein means to cause to be directed to; to transfer.

As used herein, the term “derived from” is meant to encompass any method for receiving, obtaining, or modifying something from a source of origin.

The term “diffusion” and its other grammatical forms as used herein refers to the spontaneous mixing of one substance with another when in contact or separated by a permeable membrane or microporous barrier. The rate of diffusion is proportional to the concentration of the substances and increases with temperature. Diffusion occurs most readily in gasses, less so in liquids and least in solids.

The term “disperse” as used herein means to distribute widely.

The term “dispersion”, as used herein, refers to a two-phase system, in which one phase is distributed as droplets in the second, or continuous phase. In these systems, the dispersed phase frequently is referred to as the discontinuous or internal phase, and the continuous phase is called the external phase and comprises a continuous process medium. For example, in course dispersions, the particle size is 0.5 μm. In colloidal dispersions, size of the dispersed particle is in the range of approximately 1 nm to 0.5 μm. A molecular dispersion is a dispersion in which the dispersed phase consists of individual molecules; if the molecules are less than colloidal size, the result is a true solution.

The term “disposed”, as used herein, refers to being placed, arranged or distributed in a particular fashion.

The term “elasticity” as used herein refers to a measure of the deformation of an object when a force is applied. Objects that are very elastic like rubber have high elasticity and stretch easily.

The term “extracellular vesicles” or “EVs”) as used herein refers to a heterogeneous group of cell-derived membranous structures comprising exosomes (30-200 nm) and microvesicles [0.1-1 μm], which originate from the endosomal system or which are shed from the plasma membrane of a cell, respectively. They are present in biological fluids and are involved in multiple physiological and pathological processes. EVs contain specific biomolecules, including proteins, microRNAs, mRNAs, long noncoding RNAs, cytokines, growth factors, and bioactive lipids. Some of these biomolecules indicate the vesicle origin, and others are involved in targeting cells. Extracellular vesicles are now considered as an additional mechanism for intercellular communication, allowing cells to exchange proteins, lipids and genetic material. [Doyle, L M and Wang, MZ. Overview of extracellular vesicles, their origin, composition, purpose and methods for exosome isolation and analysis. Cells (2019) 8 (7): 727; van Niel, G. et al. Shedding light on the cell biology of extracellular vesicles. Nature Reviews Molec. Cell Biol. (2018) 19: 213-28].

The term “extracellular matrix” (or “ECM”) as used herein refers to a complex network of polysaccharides and proteins secreted by cells that serves as a structural element in tissues and also influences their development and physiology. It is composed of an interlocking mesh of fibrous proteins and glycosaminoglycans (GAGs). Examples of fibrous proteins found in the extracellular matrix include collagen, elastin, fribronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached one or more glycosaminoglycans. The extracellular matrix serves many functions, including, but not limited to, providing support and anchorage for cells, segregating one tissue from another tissue, and regulating intracellular communication.

The term “fibroblast” as used herein refers to a common cell type in connective tissue that secretes an extracellular matrix rich in collagen and other extracellular matrix macromolecules and that migrates and proliferates readily in wounded tissue and in tissue culture. They have been described as plastic-adherent mesenchymal cells that play a significant role in tissue development, maintenance and repair. They are identified by their characteristic elongated, spindle-shaped morphology, along with the presence of mesenchymal markers, coupled with the absence of markers of other lineages, such as epithelial and hematopoietic markers. Fibroblasts have differentiation capacities, especially into the osteogenic, chondrogenic and adipogenic lineages. [Soundararajan, M. and Kannan, S. Fibroblasts and mesenchymal stem cells: Two sides of the same coin” J. Cellular Physiol. (2018) 233: 9099-9109, citing Blasi, A. et al Vascular Cell (2011) 3 (1): 5; Lorenz, K. et al Experimental Dermatol. (2008) 17 (11): 925-32; Sabatini, F. et al (2005) Lab. Investigation (2005) 85 (8): 962-71] and have widely different gene expression profiles [Id., citing Chang, H Y et al Proc. Natl Acad. Sci. USA (2002) 99 (20): 12877-82].

The term “flexible” as used herein refers to a material that is pliant, not stiff, and can be bent or is capable of being turned or forced from a straight line or form without breaking.

The term “fragment” or “peptide fragment” as used herein refers to a small part derived, cut off, or broken from a larger peptide, polypeptide or protein, which retains the desired biological activity of the larger peptide, polypeptide or protein.

The term “fusion protein” as used herein refers to a protein or polypeptide constructed by combining multiple protein domains or polypeptides for the purpose of creating a single polypeptide or protein with functional properties derived from each of the original proteins or polypeptides. Creation of a fusion protein may be accomplished by operatively ligating or linking two different nucleotides sequences that encode each protein domain or polypeptide via recombinant DNA technology, thereby creating a new polynucleotide sequences that codes for the desired fusion protein. Alternatively, a fusion protein maybe created by chemically joining the desired protein domains.

The term “graft” as used herein refers to transplanting or implanting tissue surgically into a body part to replace a damaged part or to compensate for a defect.

The term “growth” as used herein refers to a process of becoming larger, longer or more numerous, or an increase in size, number, or volume of cells in a cell population.

The term “growth factor” as used herein refers to an extracellular polypeptide signal molecule that that bind to a cell-surface receptor triggering an intracellular signaling pathway, leading to proliferation, differentiation, or other cellular response. Examples include BMPs, EGF, FGF, HGF, IGF-1, PDGF, TGF-β, and VEGFs.

Bone Morphogenetic Proteins.

BMPs constitute a large subclass of the TGF-β superfamily essential for normal appendicular skeletal and joint development. [Mary B. Goldring, . . . Miguel Otero, in Kelley and Firestein's Textbook of Rheumatology (Tenth Edition), 2017,]. The isolation and cloning of the first BMP family members from bone prompted a search for cartilage-derived BMPs, or CDMPs—CDMP-1, -2, and -3—which are classified as GDF-5, -6, and -7. The BMPs may be divided into four distinct subfamilies based on the similarity of primary amino acid sequences:
1. BMP-2 and -2B (BMP-4), which are 92% identical in the 7-cysteine region
2. BMP-3 (osteogenic) and -3B (GDF-10)
3. BMP-5, -6, -7 (OP-1), BMP-8 (OP-2), BMP-9 (GDF-2), BMP-10, and BMP-11 (GDF-11)
4. BMP-12 (GDF-7 or CDMP-3), BMP-13 (GDF-6 or CDMP-2), BMP-14 (GDF-5 or CDMP-1), and BMP-15.

BMP-1 is not a member of this family but is an astacin-related matrix metalloproteinase (MMP) that cleaves the BMP inhibitor chordin and acts as a procollagen C-proteinase.

Several BMPs, including BMP-2, -7 (OP-1), and GDF-5/CDMP-1, can stimulate differentiation of mesenchymal precursors into chondrocytes and promote the differentiation of hypertrophic chondrocytes. BMP-2, -4, -6, -7, -9, and -13 can enhance the synthesis of type II collagen and aggrecan (the major proteoglycan in the articular cartilage) by articular chondrocytes in vitro. BMP-2 also is expressed in normal and osteoarthritis (OA) articular cartilage, and it is a molecular marker, along with type II collagen and fibroblast growth factor receptor 3 (FGFR3), for the capacity of adult articular chondrocyte cultures to form stable cartilage in vivo. BMP-7 is expressed in mature articular cartilage and is possibly the strongest anabolic stimulus for adult chondrocytes in vitro, because it increases aggrecan and type II collagen synthesis more strongly than IGF-I. In addition, BMP-7 reverses many of the catabolic responses induced by IL-1β, including induction of MMP-1 and -13, downregulation of tissue inhibitors of metalloproteinases (TIMPs), and downregulation of proteoglycan synthesis in primary human articular chondrocytes. Cartilage derived morphogenetic proteins, also known as growth and differentiation factors (GDFs) are a subgroup of the BMP gene family. CDMP-2 is found in articular cartilage, skeletal muscle, placenta, and hypertrophic chondrocytes of the epiphyseal growth plate. CDMP-1 and -2 maintain the synthesis of type II collagen and aggrecan in mature articular chondrocytes, although they are less effective initiators of chondrogenesis than other BMPs in early progenitor cell populations in vitro.

BMPs have pleiotropic effects in vivo, however, acting in a concentration-dependent manner. While initiating chondrogenesis in the limb bud, they generally set the stage for bone morphogenesis. Several BMPs also are true morphogens for other tissues, such as kidney, eye, heart, and skin.

Epidermal growth factor (EGF). EGF is a low molecular weight polypeptide (molecular weight 6.2 KDa) with mitogenic properties (Carpenter, G. and Cohen, S. J. Biol. Chem. (1990) 265 (14): 7709-12, 1990). It has the ability to control migration, proliferation, and differentiation of fibroblasts, keratinocytes, and endothelial cells. EGFs can bind to the EGF receptor and trigger specific signaling pathways. They are widely used in skin regeneration, especially in wound healing, due to their ability to promote reepithelialization (Fatimah, S S et al., J. Biosci. Bioeng. (2012) 220-7; Imanishi, J. et al. Prog. Retin. Eye Res. (2000) 19 (1): 113-29; Bodnar, R. Adv. Wound Care (2013) 2 (1): 24-29). EGF and EGF receptor (EGFR) play an essential role in wound healing through stimulating epidermal and dermal regeneration. In addition, EGFR inhibitors (EGFRis) have become a therapeutic option for the treatment of cancer. Thus, therapies targeting EGF/EGFR are useful for the treatment of both cutaneous wounds and cancer. (Bodnar, R. Adv. Wound Care (2013) 2 (1): 24-29). Other applications include corneal repair (I Imanishi, J. et al. Prog. Retin. Eye Res. (2000) 19 (1): 113-29) and intestinal regeneration (Maeng, J H et al. J. Mater. Sci. Mater. Med. (2014) 25 (2): 573-82).

EGF is up-regulated early in the fetal period and is thought to be an important cytokine in scarless fetal healing. [Peled Z M, Rhee S J, Hsu M, Chang J, Krummel T M, Longaker M T. The ontogeny of scarless healing II: EGF and PDGF-B gene expression in fetal rat skin and fibroblasts as a function of gestational age. Ann Plast Surg. 2001 October 47 (4):417-24].

Fibroblast Growth Factor (FGF). The fibroblast growth factor (FGF) family currently has over a dozen structurally related members. FGF1 is also known as acidic FGF; FGF2 is sometimes called basic FGF (bFGF); and FGF7 sometimes goes by the name keratinocyte growth factor. Over a dozen distinct FGF genes are known in vertebrates; they can generate hundreds of protein isoforms by varying their RNA splicing or initiation codons in different tissues. FGFs can activate a set of receptor tyrosine kinases called the fibroblast growth factor receptors (FGFRs). Receptor tyrosine kinases are proteins that extend through the cell membrane. The portion of the protein that binds the paracrine factor is on the extracellular side, while a dormant tyrosine kinase (i.e., a protein that can phosphorylate another protein by splitting ATP) is on the intracellular side. When the FGF receptor binds an FGF (and only when it binds an FGF), the dormant kinase is activated, and phosphorylates certain proteins within the responding cell, activating those proteins.

FGFs are associated with several developmental functions, including angiogenesis (blood vessel formation), mesoderm formation, and axon extension. While FGFs often can substitute for one another, their expression patterns give them separate functions. For example, FGF2 is especially important in angiogenesis, whereas FGF8 is involved in the development of the midbrain and limbs.

Hepatic Growth Factor (HGF). Hepatocyte growth factor is a protein secreted by mesenchymal stem cells that regulates cell growth, cell motility and morphogenesis of epithelial cells, endothelial cells, and hematopoietic stem cells, through its receptor, c-Met. Signalling downstream of c-Met leads to cell identity changes that take place during organ development, angiogenesis and other morphogenesis processes. Rat bone marrow-derived MSCs successfully transfected to express HGF showed increased MSC viability and inhibition of the proinflammatory phenotype of MSCs in the inflammatory condition. In a rat model of ischemia/reperfusion-induced lung injury, HGF was found to contribute to the survival of engrafted MSCs in lung tissue through upregulation of Bcl-2 level and reduction of Caspase 3 activation. [Chen, S. et al. Hepatocyte growth factor-modified mesenchymal stem cells improve ischemia/reperfusion-induced acute lung injury in rats. Gene Therapy (2017) 24: 3-11].

Insulin-Like Growth Factor (IGF-1). IGF-1, a hormone similar in molecular structure to insulin, has growth-promoting effects on almost every cell in the body, especially skeletal muscle, cartilage, bone, liver, kidney, nerves, skin, hematopoietic cell, and lungs. It plays an important role in childhood growth and continues to have anabolic effects in adults. IGF-1 is produced primarily by the liver as an endocrine hormone as well as in target tissues in a paracrine/autocrine fashion. Production is stimulated by growth hormone and can be retarded by undernutrition, growth hormone insensitivity, lack of growth hormone receptors, or failures of the downstream signaling molecules, including tyrosine-protein phosphatase non-receptor type 11 (also known as SHP2, which is encoded by the PTPN11 gene in humans) and signal transducer and activator of transcription 5B (STAT5B), a member of the STAT family of transcription factors. Its primary action is mediated by binding to its specific receptor, the Insulin-like growth factor 1 receptor (IGF1R), present on many cell types in many tissues. Binding to the IGF1R, a receptor tyrosine kinase, initiates intracellular signaling; IGF-1 is one of the most potent natural activators of the AKT signaling pathway, a stimulator of cell growth and proliferation, and a potent inhibitor of programmed cell death. IGF-1 is a primary mediator of the effects of growth hormone. Growth hormone is made in the pituitary gland, released into the blood stream, and then stimulates the liver to produce IGF-1. IGF-1 then stimulates systemic body growth. In addition to its insulin-like effects, IGF-1 also can regulate cell growth and development, especially in nerve cells, as well as cellular DNA synthesis.

IGF-1 was shown to increase the expression levels of the chemokine receptor CXCR4 (receptor for stromal cell-derived factor-1, SDF-1) and to markedly increase the migratory response of MSCs to SDF-1 (Li, Y, et al. 2007 Biochem. Biophys. Res. Communic. 356(3): 780-784). The IGF-1-induced increase in MSC migration in response to SDF-1 was attenuated by PI3 kinase inhibitor (LY294002 and wortmannin) but not by mitogen-activated protein/ERK kinase inhibitor PD98059. Without being limited by any particular theory, data indicate that IGF-1 increases MSC migratory responses via CXCR4 chemokine receptor signaling which is PI3/Akt dependent.

Platelet-derived growth factor (PDGFs). PDGFs are disulfide-bonded heterodimeric proteins that act mainly on stromal cells and regulate the wound healing process. Originally isolated from platelets, PDGF isoforms are also secreted from macrophages, endothelial cells, and fibroblasts. PDGF, signaling via the α and β transmembrane receptors, acts as a potent mitogen (stimulator of cell division) and chemoattractant for fibroblasts. Moreover, PDGF induces ROS generation and stimulates the synthesis of collagen, fibronectin, and proteoglycans, as well as the secretion of TGF-β1, MCP-1, and IL-6. Overactivity of PDGF has been linked to certain diseases, such as malignancies in which PDGF production may promote tumor growth via autocrine or paracrine stimulation. PDGF is also implicated in other disorders that involve an excess of cell proliferation, e.g., atherosclerosis and fibrotic conditions.

Transforming Growth Factor Beta (TGF-β). There are over 30 structurally related members of the TGF-β superfamily, and they regulate some of the most important interactions in development. The proteins encoded by TGF-β superfamily genes are processed such that the carboxy-terminal region contains the mature peptide. These peptides are dimerized into homodimers (with themselves) or heterodimers (with other TGF-β peptides) and are secreted from the cell. The TGF-β superfamily includes the TGF-β family, the activing family, BMPs, the Vg-1 family, and other proteins, including glial-derived neurotrophic factor (GDNF, necessary for kidney and enteric neuron differentiation) and Müllerian inhibitory factor, which is involved in mammalian sex determination. TGF-β family members TGF-β1, 2, 3, and 5 are important in regulating the formation of the extracellular matrix between cells and for regulating cell division (both positively and negatively). TGF-β1 increases the amount of extracellular matrix epithelial cells make both by stimulating collagen and fibronectin synthesis and by inhibiting matrix degradation. TGF-βs may be critical in controlling where and when epithelia can branch to form the ducts of kidneys, lungs, and salivary glands.

Vascular Endothelial Growth Factor (VEGF). VEGFs are growth factors that mediate numerous functions of endothelial cells including proliferation, migration, invasion, survival, and permeability. The VEGFs and their corresponding receptors are key regulators in a cascade of molecular and cellular events that ultimately lead to the development of the vascular system, either by vasculogenesis, angiogenesis, or in the formation of the lymphatic vascular system. VEGF is a critical regulator in physiological angiogenesis and also plays a significant role in skeletal growth and repair.

VEGF's normal function creates new blood vessels during embryonic development, after injury, and to bypass blocked vessels. In the mature established vasculature, the endothelium (a monolayer of cells that lines the entire inner surface of the cardiovascular and lymphatic circulations where it controls normal physiological functions through both systemic and local regulation) plays an important role in the maintenance of homeostasis of the surrounding tissue by providing the communicative network to neighboring tissues to respond to requirements as needed. Furthermore, the vasculature provides growth factors, hormones, cytokines, chemokines and metabolites, and the like, needed by the surrounding tissue and acts as a barrier to limit the movement of molecules and cells.

The terms “healthy subject” or “normal healthy subject” are used herein interchangeably to refer to a subject having no symptoms or other evidence of a periodontal inflammatory disorder, e.g., periodontitis.

The term “hydrogel” as used herein refers to a substance resulting in a solid, semisolid, pseudoplastic, or plastic structure containing a necessary aqueous component to produce a gelatinous or jelly-like mass. Hydrogels are an appealing scaffold material because they are structurally similar to the extracellular matrix of many tissues, can often be processed under relatively mild conditions, and may be delivered in a minimally invasive manner. [Drury, J L and Mooney, DJ. Hydrogels for Tissue Engineering: scaffold design variables and applications. Biomaterials (2003) 24 (24): 4337-51]. Their structural integrity depends on crosslinks formed between polymer chains via various chemical bonds and physical interactions.

For hydrogels, there are three basic degradation mechanisms: hydrolysis, enzymatic cleavage, and dissolution. Most synthetic hydrogels are degraded through hydrolysis of ester linkages [Id., citing Metters, A T et al. Fundamental studies of a novel, biodegradable PEG-n-PLA hydrogel. Polymer (2000) 41: 3993-4004; Suggs, L J and Mikos, AG. Development of poly(propylene fumarate-co-ethylene glycol) hydrogels. J. Biomed. Mater. Res. (1999) 46: 22-32; Saito, N. et al. A biodegradable polymer as a cytokine delivery system for inducing bone formation. Nat. Biotech (2001) 12: 332-5]. Unlike solid polymers, hydrogels undergo purely bulk degradation since they are hydrated within the structures. To control the degradation of these polymers, hydrogels have been copolymerized with other polymers to introduce synthetic linkages [Kano, T. et al. Enhancement of drug solubility and absorption by copolymers of 2-methacryloyloxyethyl phosphorylcholine and n-butyl methacrylate. Drug Metab. Pharmacokinet. (2011) 26 (1): 79-86], and with peptides sensitive to enzymatic degradation, including, without limitation, collagen, hyaluronic acid, and chitosan [Drury, J L and Mooney, DJ. Hydrogels for Tissue Engineering: scaffold design variables and applications. Biomaterials (2003) 24 (24): 4337-51, citing Lee, K Y et al. Blood compatibility and biodegradability of partially N-acetylated chitosan derivatives. Biomaterials (1995) 16: 1211-26; Varum, K M et al. Determination of enzymatic hydrolysis specificity of partially N-acetylated chitosan. Biochem. Biophys. Acta (1996) 29: 5-15; Tomihata, K. and Ikada, Y. In vitro and in vivo degradation of films of chitin and its deacetylated derivatives. Biomaterials (1997) 18: 567-75].

Bulk degradation is characterized by four phases. The first phase involves swelling to equilibrium accompanied by a proportionate loss of modulus (meaning a coefficient expressing a specified property of a specified substance. For example, Young's modulus (see below) is a measure of the stiffness of a material, i.e., how easily it is bent or stretched). The second phase involves the gradual cleavage of hydrolytically labile bonds with a corresponding growth in volume and additional loss of modulus and strength. The third phase, actually a continuation of the second phase, shows an acceleration of swelling as the gel's molecular network approaches disintegration. The final phase involves mass and volume loss until complete dissolution occurs. [Jarrett, P. and Coury, A. Tissue adhesives and sealants for surgical applications, Chapter 16 in Joining and assembly of Medical Materials and devices (2013) pp. 449-490].

The term “hydrophilic” as used herein refers to a material or substance having an affinity for polar substances, such as water.

The terms “immune response” and “immune mediated” are used interchangeably herein to refer to any functional expression of a subject's immune system against either foreign or self-antigens, whether the consequences of these reactions are beneficial or harmful to the subject.

The term “immune system” as used herein refers to a complex arrangement of cells and molecules that maintain immune homeostasis (meaning maintaining a balance between immune tolerance and immunogenicity) to preserve the integrity of the organism by elimination of all elements judged to be dangerous. Responses in the immune system may generally be divided into two arms, referred to as “innate immunity” and “adaptive immunity.” The two arms of immunity do not operate independently of each other, but rather work together to elicit effective immune responses.

The term “implant” as used herein refers to a material inserted or grafted on or into a tissue.

The term “impregnate” as used herein in its various grammatical forms refers to causing to be infused or permeated throughout, or to fill interstices with a substance.

The terms “innate immunity” or “innate immune response” are used interchangeably to refer to a nonspecific fast response to pathogens that is predominantly responsible for an initial inflammatory response via a number of soluble factors, including the complement system and the chemokine/cytokine system; and a number of specialized cell types, including mast cells, macrophages, dendritic cells (DCs), and natural killer cells (NKs).

The term “integrins” as used herein refers to the principal receptors used by animal cells to bind to the extracellular matrix. They are heterodimers and function as transmembrane linkers between the extracellular matrix and the actin cytoskeleton. A cell can regulate the adhesive activity of its integrins from within.

The term “interlaced” and its other grammatical forms as used herein refers to a state of being united by intercrossing; of being passed over and under each other; of being weaved together; intertwined; or being connected intricately.

The term “isolated” as used herein refers to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95% free of, more than about 96% free of, more than about 97% free of, more than about 98% free of, or more than about 99% free of such components. The isolated material optionally comprises (a) material not found with the material in its natural environment; or (b) the material has been synthetically (non-naturally) altered by deliberate human intervention.

The term “matrix” as used herein refers to a three-dimensional network of fibers that contains voids (or “pores”) where the woven fibers intersect. The structural parameters of the pores, including the pore size, porosity, pore interconnectivity/tortuosity and surface area, can affect how substances (e.g., fluid, solutes) move in and out of the matrix.

The term “matrix metalloproteinases” or “MMPs” as used herein refers to a large family of calcium-dependent zinc-containing endopeptidases, which are involved in the tissue remodeling and degradation of the extracellular matrix.

Mesenchymal Stem Cells. Mesenchymal stem cells (MSCs) (also known as bone marrow stromal stem cells or skeletal stem cells) are non-blood adult stem cells found in a variety of tissues. They are characterized by their spindle-shape morphologically; by the expression of specific markers on their cell surface; and by their ability, under appropriate conditions, to differentiate along a minimum of three lineages (osteogenic, chondrogenic, and adipogenic) [Najar M. et al., “Mesenchymal stromal cells and immunomodulation: A gathering of regulatory immune cells”, Cytotherapy, Vol. 18(2): 160-171, (2016)]. No single marker that definitely delineates MSCs in vivo has been identified due to the lack of consensus regarding the MSC phenotype. The International Society for Cellular Therapy (ISCT) in 2006 provided the following set of minimal criteria to describe a cell as a multipotent MSC: (1) the cells must be plastic adherent when maintained under standard conditions; (2) they must express CD105, CD73, and CD90, and lack expression of CD45, CD34, CD14 or CD11b, CD79a, or CD19, and HLA-DR surface molecules; and (3) the MSCs must differentiate into osteoblasts, adipocytes, and chondrocytes in vitro. [Dominici, M. et al. Minimal criteria for defining multipotent mesenchymal stromal cells. The Intl Soc'y for Cellular Therapy position statement. Cytotherapy (2006) 8 (4): 315-17]. As for the differentiation potential of MSCs, studies have reported that populations of bone marrow-derived MSCs have the capacity to develop into terminally differentiated mesenchymal phenotypes both in vitro and in vivo, including bone, cartilage, tendon, muscle, adipose tissue, and hematopoietic supporting stroma. Studies using transgenic and knockout mice and human musculoskeletal disorders have reported that MSCs differentiate into multiple lineages during embryonic development and adult homeostasis [Najar M. et al., “Mesenchymal stromal cells and immunomodulation: A gathering of regulatory immune cells”, Cytotherapy, Vol. 18(2): 160-171, (2016)]. Analysis of the in vitro differentiation of MSCs under appropriate conditions that recapitulate the in vivo process have led to the identification of various factors essential for stem cell commitment. Among them, secreted molecules and their receptors (e.g., transforming growth factor-beta (TGF-β), extracellular matrix molecules (e.g., collagens and proteoglycans), the actin cytoskeleton, and intracellular transcription factors (e.g., Cbfa1/Runx2, PPARy, Sox9, and MEF2) have been shown to play important roles in driving the commitment of multipotent MSCs into specific lineages, and maintaining their differentiated phenotypes [Davis L. A. et al., “Mesodermal fate decisions of a stem cell: the Wnt switch”, Cell Mol Life Sci., Vol. 65(17): 2568-2574, (2008)].

The term “MPC” is an abbreviation for methacryloyloxyethyl phosphorylcholine, a zwitterionic phospholipid polymer which is a methacrylate that harbors a phospholipid polar group in its side chain, providing a highly hydrophilic surface that can resist protein absorption and bacterial adhesion. [Kwon, J-S et al. Novel anti-biofouling light-curable fluoride varnish containing 2-methacryloyloxyethyl phosphottttttrylcholine to prevent enamel demineralization. Sci. Reports (2019) 9: 1432, citing Moro, T. et al. Water resistance of artificial hip joints with poly (2methacryloyloxyethyl phosphorylcholine) grafted polyethylene: comparisons with the effect of polyethylene cross-linking and ceramic femoral heads. Biomaterials (20009) 30: 2993-3001]. In water, the MPC phospholipids will orient themselves into a bilayer in which the nonpolar tails face the inner area of the bilayer and the polar heads face outward to interact with the water, which results in its highly hydrophilic properties. [Id., citing Lewis, A. et al. Analysis of a phosphorylcholine-based polymer coating on a coronary stent pre- and post-implantation. Biomaterials (2002) 23: 1697-1706].

The term “miosis” as used herein means excessive constriction (shrinking) of the pupil. In miosis, the diameter of the pupil is less than 2 millimeters (mm),

The term “modulate” as used herein means to regulate, alter, adapt, or adjust to a certain measure or proportion.

The term “morphogen” as used herein refers to diffusible molecules produced in a restricted region of a tissue that can impart specific differentiation programs as part of a series of signaling, transcriptional and morphogenetic events in the context of various feedbacks and other inputs to target cells through the establishment of a dynamic long-range concentration gradient in space and time (both temporal window and duration), in combination with the involvement of additional cooperating signaling pathways. Therefore, to be considered a morphogen, a signaling molecule must meet two criteria: (1) it must have a concentration-dependent effect on its target cells, and (2) it must exert a direct action at a distance. [See Economou, A D and Hill, CS. Chapter 12, Temporal dynamics in the formation and interpretation of Nodal and BMP morphogen gradients. In Current Topics in Devel. Biology (2020) 137: 363-89; Huang, A. and Saunders, TE. Ch. 3, “A matter of time: formation and interpretation of the Bicoid morphogen gradient. Current Topics in Developmental Biol. (2020) 137: 79-117; Grove, E A and Monuki, E S. Ch. 2 “Morphogens, Patterning Centrs and their Mechanisms of Action” in Patterning and Cell Type Specification in the Developing CNS and PNS. Comprehensive Developmental Neuroscience, Vol. 1. Elsevier, Inc. (2013): 26-44].

The term “myeloid” as used herein means of or pertaining to bone marrow. Granulocytes and monocytes, collectively called myeloid cells, are differentiated descendants from common progenitors derived from hematopoietic stem cells in the bone marrow. Commitment to either lineage of myeloid cells is controlled by distinct transcription factors followed by terminal differentiation in response to specific colony-stimulating factors and release into the circulation. Upon pathogen invasion, myeloid cells are rapidly recruited into local tissues via various chemokine receptors, where they are activated for phagocytosis as well as secretion of inflammatory cytokines, thereby playing major roles in innate immunity. [Kawamoto, H., Minato, N. Intl J. Biochem. Cell Biol. (2004) 36 (8): 1374-9].

The term “myofibroblast” as used herein refers to a differentiated cell type essential for wound healing that participates in tissue remodeling following an insult. Myofibroblasts are typically activated fibroblasts, although they can also be derived from other cell types, including epithelial cells, endothelial cells, and mononuclear cells.

The term “osmosis” as used herein refers to tendency of a fluid to pass through a semipermeable membrane into a solution of lower concentration so as to equalize concentrations on both sides of the membrane.

The term “osmotic pressure” as used herein refers to pressure in a solution due to the presence of a dissolved substance.

The term “paracrine signaling” as used herein refers to short range cell-cell communication via secreted signal molecules that act on adjacent cells.

The term “PEGDA” is an abbreviation for poly(ethylene glycol)diacrylate, a hydrophilic copolymer.

The term “periodontal ligament” as used herein refers to the soft connective tissue between the inner wall of the alveolar socket and the roots of the teeth which consists of collagen bands (mostly type I collagen) connecting the cementum of the teeth to the gingivae and alveolar bone.

The term “permeable” as used herein means permitting the passage of substances, such as oxygen, glucose, water and ions, as through a membrane or other structure.

The term “porosity” as used herein refers to the ratio between the pore volume and the total volume of a material.

The term “peptide” as used herein refers to a molecule of two or more amino acids chemically linked together. A peptide may refer to a polypeptide, protein or peptidomimetic.

The term “peptidomimetic” refers to a small protein-like chain designed to mimic or imitate a peptide. A peptidomimetic may comprise non-peptidic structural elements capable of mimicking (meaning imitating) or antagonizing (meaning neutralizing or counteracting) the biological action(s) of a natural parent peptide.

The terms “polypeptide” and “protein” are used herein in their broadest sense to refer to a sequence of subunit amino acids, amino acid analogs, or peptidomimetics. The subunits are linked by peptide bonds, except where noted. The polypeptides described herein may be chemically synthesized or recombinantly expressed. Polypeptides of the described invention can be chemically synthesized. Synthetic polypeptides, prepared using the well-known techniques of solid phase, liquid phase, or peptide condensation techniques, or any combination thereof, can include natural and unnatural amino acids. Amino acids used for peptide synthesis may be standard Boc (N-α-amino protected N-α-t-butyloxycarbonyl) amino acid resin with the standard deprotecting, neutralization, coupling and wash protocols of the original solid phase procedure of Merrifield (1963, J. Am. Chem. Soc 85:2149-2154), or the base-labile N-α-amino protected 9-fluorenylmethoxycarbonyl (Fmoc) amino acids first described by Carpino and Han (1972, J. Org. Chem. 37:3403-3409). Both Fmoc and Boc N-α-amino protected amino acids can be obtained from Sigma, Cambridge Research Biochemical, or other chemical companies familiar to those skilled in the art. In addition, the polypeptides can be synthesized with other N-α-protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept. Protein Res. 35:161-214, or using automated synthesizers. The polypeptides of the invention may comprise D-amino acids (which are resistant to L-amino acid-specific proteases in vivo), a combination of D- and L-amino acids, and various “designer” amino acids (e.g., β-methyl amino acids, C-α-methyl amino acids, and N-α-methyl amino acids, etc.) to convey special properties. Synthetic amino acids include ornithine for lysine, and norleucine for leucine or isoleucine. In addition, the polypeptides can have peptidomimetic bonds, such as ester bonds, to prepare peptides with novel properties. For example, a peptide may be generated that incorporates a reduced peptide bond, i.e., R¹—CH₂—NH—R², where R₁ and R₂ are amino acid residues or sequences. A reduced peptide bond may be introduced as a dipeptide subunit. Such a polypeptide would be resistant to protease activity, and would possess an extended half-live in vivo. Accordingly, these terms also apply to amino acid polymers in which one or more amino acid residue is an artificial chemical analogue of a corresponding naturally occurring amino acid, as well as to naturally occurring amino acid polymers. When incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well.

The term “polymer” as used herein refers to any of various chemical compounds made of smaller, identical molecules (called monomers) linked together. Polymers generally have high molecular weights. The incorporation of two different monomers, A and B, into a polymer chain in a statistical fashion leads to copolymers. In the limit, single monomers may alternate regularly in the chain and these are known as alternating copolymers. The monomers can be combined in a more regular fashion, either by linking extended linear sequences of one to linear sequences of the other by end-to-end addition to give block copolymers, or by attaching chains of B at points on the backbone chain of A, forming a branched structure known as a graft copolymer.

The term “proliferate” and its various grammatical forms as used herein means to increase rapidly in numbers; to multiply.

The term “protein” is used herein to refer to a large complex molecule or polypeptide composed of amino acids. The sequence of the amino acids in the protein is determined by the sequence of the bases in the nucleic acid sequence that encodes it.

The term “range” and its various grammatical forms as used herein refers to varying between the stated limits and includes the stated limits and all points or values in between.

The term “recombinant DNA” refers to a DNA molecule formed by laboratory methods whereby DNA segments from different sources are joined to produce a new genetic combination.

The term “recombinant protein” as used herein refers to a protein encoded by recombinant DNA that has been cloned in a system that supports expression of the gene and translation of messenger RNA within a living cell. To make a human recombinant protein, for example, a gene of interest is isolated, cloned into an expression vector, and expressed in an expression system. Exemplary expression systems include prokaryotic organisms, as bacteria, and eukaryotic organisms, such as yeast, insect cells, plants, and mammalian cells in culture.

The term “release” and its various grammatical forms refers to dissolution and diffusion of a dissolved or solubilized species by a combination of the following processes: (1) hydration of a matrix, (2) diffusion of a solution into the matrix; (3) dissolution of the active; and (4) diffusion of the dissolved active out of the matrix.

The term “shape” as used herein refers to the quality of a distinct object or body in having an external surface or outline of specific form or figure.

The term “stem cells” as used herein refers to undifferentiated cells having high proliferative potential with the ability to self-renew that can generate daughter cells that can undergo terminal differentiation into more than one distinct cell phenotype. Stem cells are distinguished from other cell types by two characteristics. First, they are unspecialized cells capable of renewing themselves through cell division, sometimes after long periods of inactivity. Second, under certain physiologic or experimental conditions, they can be induced to become tissue- or organ-specific cells with special functions. In some organs, such as the gut and bone marrow, stem cells regularly divide to repair and replace worn out or damaged tissues. In other organs, however, such as the pancreas and the heart, stem cells only divide under special conditions.

Adult (somatic) stem cells are undifferentiated cells found among differentiated cells in a tissue or organ. Their primary role in vivo is to maintain and repair the tissue in which they are found. Adult stem cells have been identified in many organs and tissues, including brain, bone marrow, peripheral blood, blood vessels, skeletal muscles, skin, teeth, gastrointestinal tract, liver, ovarian epithelium, and testis. Adult stem cells are thought to reside in a specific area of each tissue, known as a stem cell niche, where they may remain quiescent (non-dividing) for long periods of time until they are activated by a normal need for more cells to maintain tissue, or by disease or tissue injury.

The terms “subject” or “individual” or “patient” are used interchangeably to refer to a member of an animal species of mammalian origin, including but not limited to, mouse, rat, cat, goat, sheep, horse, hamster, ferret, pig, dog, guinea pig, rabbit and a primate, such as, for example, a monkey, ape, or human.

The term “thickness” as used herein refers to a measure between opposite surfaces, from top to bottom, or in a direction perpendicular to that of the length and breadth.

The term “tolerance limits” as used herein refers to the end points of a tolerance interval (meaning a confidence interval covering a specified proportion of the population with a stated confidence, i.e., a certain proportion of the time).

The term “transactivation” as used herein refers to stimulating transcription of a gene in a host cell by binding to DNA. Genes can be transactivated naturally (e.g., by a virus or a cellular protein) or artificially.

The terms “variants”, “mutants”, and “derivatives” are used herein to refer to nucleotide sequences with substantial identity to a reference nucleotide sequence. The differences in the sequences may by the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

A skilled artisan likewise can produce polypeptide variants having single or multiple amino acid substitutions, deletions, additions or replacements. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, such as, for example, an epitope for an antibody. The techniques for obtaining such variants, including genetic (suppressions, deletions, mutations, etc.), chemical, and enzymatic techniques are known to the skilled artisan. As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “viscosity”, as used herein refers to the property of a fluid that resists the force tending to cause the fluid to flow. Viscosity is a measure of the fluid's resistance to flow. The resistance is caused by intermolecular friction exerted when layers of fluids attempt to slide by one another. Viscosity can be of two types: dynamic (or absolute) viscosity and kinematic viscosity. Absolute viscosity or the coefficient of absolute viscosity is a measure of the internal resistance. Dynamic (or absolute) viscosity is the tangential force per unit area required to move one horizontal plane with respect to the other at unit velocity when maintained a unit distance apart by the fluid. Dynamic viscosity is usually denoted in poise (P) or centipoise (cP), wherein 1 poise=1 g/cm², and 1 cP=0.01 P. Kinematic viscosity is the ratio of absolute or dynamic viscosity to density. Kinematic viscosity is usually denoted in Stokes (St) or Centistokes (cSt), wherein 1 St=10-4 m²/s, and 1 cSt=0.01 St.

The term “wt %” or “weight percent” or “percent by weight” or “wt/wt %” of a component, unless specifically stated to the contrary, refers to the ratio of the weight of the component to the total weight of the composition in which the component is included, expressed as a percentage.

The term “Young's modulus” as used herein refers to a measure of elasticity, equal to the ratio of the stress acting on a substance to the strain produced. The term “stress” as used herein refers to a measure of the force put on an object over an area. The term “strain” as used herein refers to the change in length divided by the original length of the object. Change in length is proportional to the force put on it and depends on the substance from which the object is made. Change in length is proportional to the original length and inversely proportional to the cross-sectional area. Fracture is caused by a strain placed on an object such that it deforms (a change of shape) beyond its elastic limit and breaks.

### EMBODIMENTS

### Hydrogel Composition

According to one aspect, the present invention disclosure provides a hydrogel composition comprising an interpenetrating polymer network (IPN) containing a biopolymer, a first synthetic polymer and a second synthetic polymer. Although discussed herein with respect to an inlay, it should be understood that the hydrogel composition can be used to formulate a hydrogel polymer capable of receiving and supporting live cells and their released cell products (e.g., a hydrogel polymer scaffold for treatment of gum disease, or the like).

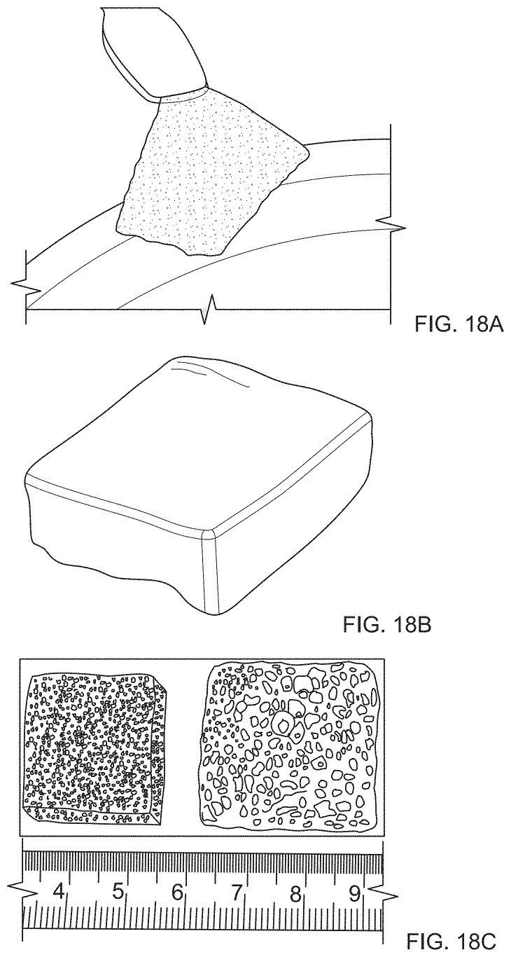

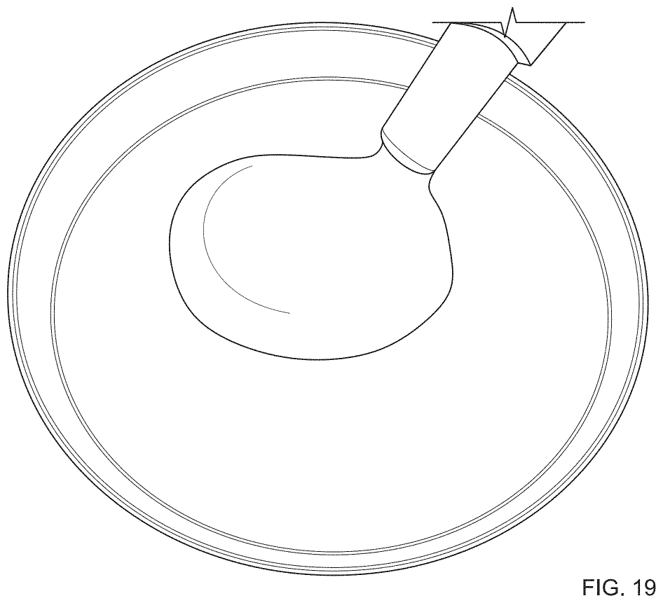

FIGS. 18A, 18B, and 18C are images of some collagen implants in the form of scaffolds that have been suggested for gum disease treatment, and FIG. 19 is an image of an injectable hydrogel scaffold that has been suggested for gum disease treatment.

An IPN is a polymer comprising two or more networks that are at least partially interlaced on a molecule scale but not covalently bonded to each other and cannot be separated unless chemical bonds are broken (Intl Union of Pure & Applied Chemistry Compendium of Chemical Terminology (IUPAC Gold Book, v. 2.3.3 (2014-02-24), page 750). Mixtures of two or more polymers cannot be termed IPNs and are instead multicomponent polymer material.

The main advantage of IPNs is their mechanical strength and stability. Also IPNs provide an opportunity to have two or more polymers with distinguishing properties. By modifying the interaction of the IPNs, a synergy can be achieved, which results in enhanced performance that surpasses that of either of the original polymers. (Purkait, M K, et al. Interface Science and Technology (2018) vol. 25, chapter 3, section 3.2.3: 67-113).

For the preparation of an IPN hydrogel, the polymers should meet the following criteria. First, there should be one polymer which can be synthesized and/or cross-linked with the other. Second, the polymers should have similar reaction rates. Lastly, there should not be any phase separation between/among the polymers (Id., citing (Bajpai, A K et al. Responsive polymers in controlled drug delivery. Prog. Polym. Sci. (2008) 33: 1088-18). Advantages of IPN hydrogels include their viscoelastic properties and easy swelling behavior without dissolving in any solvent (Id.). IPNs can be prepared (i) by chemistry and (ii) by structure [Id., citing Myung, D. et al. Polym. Adv. Technol. (2008) 647-57; Naseri, N. et al. Biomacromolecules (2016) 17: 3714-23.

Depending on the chemistry of preparation, IPN hydrogels can be divided into simultaneous IPNs or sequential IPNs. In simultaneous IPNs, both the networks are prepared simultaneously from the precursors by independent, noninterfering routes that will not interfere with one another. In sequential IPNs, a network is made of a single network hydrogel by swelling into a solution comprising the mixture of monomer, initiator and activator, with or without a cross-linker. (Id.).

Depending on the structure, IPN hydrogels can be categorized into the following types:
(a) full IPNs which are composed of two networks that are ideally juxtaposed, with many entanglements and interactions between the networks;
(b) homo-IPNs, where the two polymers used in the networks are the same;
(c) Semi- or pseudo-IPNs, which is a way of blending of two polymers, where one is cross-linked in the presence of the other to produce a mixture of fine morphology; additional noncovalent interaction between the two polymers can influence the surface morphology and the thermal properties of the semi-IPN gel;
(d) latex IPNs, which result from emulsion polymerization. The morphology of the latex IPN depends on the polymerization techniques of the IPN components;
(e) thermoplastic IPNs, which can be moldable, extruded and recycled. At least one component generally is a block copolymer. (Id.).

In some embodiments, the hydrogel polymer composition comprises an interpenetrating network (IPN) which includes two or more polymeric units in the network in which the polymers are interlaced with each other (Maity, S. et al. Green approaches in medicinal chemistry for sustainable drug design. Advances in Green Chemistry (2020) 617-49, citing Dragan, E. S. “Design and applications of interpenetrating polymer network hydrogels. A review. Chem. Eng. J. (2014) 243: 572-90). The IPN provides mechanical strength and stability to the implant formed from the hydrogel composition, thereby providing robustness sufficient to withstand surgical procedure(s), if needed.

In some embodiments, the hydrogel composition comprises a biopolymer. In some embodiments, the natural biopolymer is a collagen. In some embodiments, the natural biopolymer is different from the collagen. In some embodiments, the percentage by weight of the natural polymer within the hydrogel composition can be substantially equal to the percentage by weight of the collagen. In some embodiments, the natural polymer can be a collagen. In some embodiments, the biopolymer is a synthetic self-assembling biopolymer. In some embodiments, the biopolymer is a naturally-occurring biopolymer. Exemplary naturally-occurring biopolymers include, but are not limited to, protein polymers, collagen, polysaccharides, and photopolymerizable compounds. Exemplary protein polymers synthesized from self-assembling protein polymers include, for example, silk fibroin, elastin, collagen, and combinations thereof. In some embodiments, the biopolymer comprises a collagen. In some embodiments, the collagen is a porcine collagen. In some embodiments, the collagen is a recombinant collagen. In some embodiments, a synthetic self-assembling biopolymer is a synthetic collagen. In some embodiments, the synthetic self-assembling biopolymer is a recombinant human collagen. In some embodiments, the collagen is a collagen mimetic peptide. As used herein, the term “mimetic” refers to chemicals containing chemical moieties that mimic the function of a peptide. For example, if a peptide contains two charged chemical moieties having functional activity, a mimetic places two charged chemical moieties in a spatial orientation and constrained structure so that the charged chemical function is maintained in three-dimensional space.

In some embodiments, the hydrogel composition comprises a synthetic polymeric material. In some embodiments, the synthetic material is a biocompatible material. In some embodiments, the synthetic material is a biodegradable material. In some embodiments, the synthetic material is a hydrophilic material. In some embodiments, the synthetic materials is a material permeable to low molecular weight nutrients so as to maintain tissue health. In some embodiments, the synthetic material is moldable, biocompatible, hydrophilic, and permeable.

In some embodiments, the first synthetic polymer and the second synthetic polymer are the same. In some embodiments, the first synthetic polymer and the second synthetic polymer are different.

In some embodiments, the first and second synthetic polymer have at least one different property. A wide variety of properties can be different among the polymers, including, without limitation chemical composition, viscosity (e.g., intrinsic viscosity), molecular weight, thermal properties, such as glass transition temperature (Tg), the chemical composition of a non-repeating unit therein, such as an end group, degradation profile, hydrophilicity, porosity, density, or a combination thereof.

In some embodiments, the first and second synthetic polymer have one or more different non-repeating units, such as, for example, an end group, or a non-repeating unit in the backbone of the polymer. In a further aspect, the first polymer and the second polymer of the polymer matrix have one or more different end groups. For example, the first polymer can have a more polar end group than one or more end group(s) of the second polymer.

In some embodiments, the first synthetic polymer and the second synthetic polymer have different molecular weights. The molecular weight can have any suitable value, which can, in various aspects, depend on the desired properties of the IPN and the composition.

In some embodiments, the ratio of the first synthetic polymer to the second synthetic polymer can be present in any desired ratio, which is the weight ratio of the first synthetic polymer to the second synthetic polymer. In addition, more than two synthetic polymers, or biosynthetic polymers can be present in a blend.

In some embodiments, the water content of the hydrogel composition can range from 40%-92% (w/w), inclusive. In some embodiments, the water content is at least 40%. In some embodiments, the water content is at least 41%. In some embodiments, the water content is at least 42%. In some embodiments, the water content is at least 43%. In some embodiments, the water content is at least 44%. In some embodiments, the water content is at least 45%. In some embodiments, the water content is at least 46%. In some embodiments, the water content is at least 47%. In some embodiments, the water content is at least 48%. In some embodiments, the water content is at least 49%. In some embodiments, the water content is at least 50%. In some embodiments, the water content is at least 51%. In some embodiments, the water content is at least 52%. In some embodiments, the water content is at least 53%. In some embodiments, the water content is at least 54%. In some embodiments, the water content is at least 55%. In some embodiments, the water content is at least 56%. In some embodiments, the water content is at least 57%. In some embodiments, the water content is at least 58%. In some embodiments, the water content is at least 59%. In some embodiments, the water content is at least 60%. In some embodiments, the water content is at least 61%. In some embodiments, the water content is at least 62%. In some embodiments, the water content is at least 63%. In some embodiments, the water content is at least 64%. In some embodiments, the water content is at least 65%. In some embodiments, the water content is at least 66%. In some embodiments, the water content is at least 67%. In some embodiments, the water content is at least 68%. In some embodiments, the water content is at least 69%. In some embodiments, the water content is at least 70%. In some embodiments, the water content is at least 71%. In some embodiments, the water content is at least 72%. In some embodiments, the water content is at least 73%. In some embodiments, the water content is at least 74%. In some embodiments, the water content is at least 75%. In some embodiments, the water content is at least 76%. In some embodiments, the water content is at least 77%. In some embodiments, the water content is at least 78%. In some embodiments, the water content is at least 79%. In some embodiments, the water content is at least 80%. In some embodiments, the water content is at least 81%. In some embodiments, the water content is at least 82%. In some embodiments, the water content is at least 83%. In some embodiments, the water content is at least 84%. In some embodiments, the water content is at least 85%. In some embodiments, the water content is at least 86%. In some embodiments, the water content is at least 87%. In some embodiments, the water content is at least 88%. In some embodiments, the water content is at least 89%. In some embodiments, the water content is at least 90%. In some embodiments, the water content is at least 91%. In some embodiments, the water content is at least 92%.

In some embodiments, the water content of the hydrogel composition is less than 92%. In some embodiments, the water content is less than 90%. In some embodiments, the water content is less than 88%. In some embodiments, the water content ranges from at least 40%-91%, inclusive. In some embodiments, the water content ranges from at least 40%-91%, inclusive. In some embodiments, the water content ranges from at least 40%-90%, inclusive. In some embodiments, the water content ranges from at least 40%-89%, inclusive. In some embodiments, the water content ranges from at least 40%-88%, inclusive. In some embodiments, the water content ranges from at least 40%-87%, inclusive. In some embodiments, the water content ranges from at least 40%-86%, inclusive. In some embodiments, the water content ranges from at least 40%-85%, inclusive. In some embodiments, the water content ranges from at least 40%-84%, inclusive. In some embodiments, the water content ranges from at least 40%-83%, inclusive. In some embodiments, the water content ranges from at least 40%-82%, inclusive. In some embodiments, the water content ranges from at least 40%-81%, inclusive. In some embodiments, the water content ranges from at least 40%-80%, inclusive. In some embodiments, the water content ranges from at least 40%-79%, inclusive. In some embodiments, the water content ranges from at least 40%-78%, inclusive. In some embodiments, the water content ranges from at least 40%-77%, inclusive. In some embodiments, the water content ranges from at least 40%-76%, inclusive. In some embodiments, the water content ranges from at least 40%-75%, inclusive. In some embodiments, the water content ranges from at least 40%-74%, inclusive. In some embodiments, the water content ranges from at least 40%-73%, inclusive. In some embodiments, the water content ranges from at least 40%-72%, inclusive. In some embodiments, the water content ranges from at least 40%-71%, inclusive. In some embodiments, the water content ranges from at least 40%-70%, inclusive. In some embodiments, the water content ranges from at least 40%-69%, inclusive. In some embodiments, the water content ranges from at least 40%-68%, inclusive. In some embodiments, the water content ranges from at least 40%-67%, inclusive. In some embodiments, the water content ranges from at least 40%-66%, inclusive. In some embodiments, the water content ranges from at least 40%-65%, inclusive. In some embodiments, the water content ranges from at least 40%-64%, inclusive. In some embodiments, the water content ranges from at least 40%-63%, inclusive. In some embodiments, the water content ranges from at least 40%-62%, inclusive. In some embodiments, the water content ranges from at least 40%-61%, inclusive. In some embodiments, the water content ranges from at least 40%-60%, inclusive. In some embodiments, the water content ranges from at least 40%-59%, inclusive. In some embodiments, the water content ranges from at least 40%-58%, inclusive. In some embodiments, the water content ranges from at least 40%-57%, inclusive. In some embodiments, the water content ranges from at least 40%-56%, inclusive. In some embodiments, the water content ranges from at least 40%-55%, inclusive. In some embodiments, the water content ranges from at least 40%-54%, inclusive. In some embodiments, the water content ranges from at least 40%-53%, inclusive. In some embodiments, the water content ranges from at least 40%-52%, inclusive. In some embodiments, the water content ranges from at least 40%-51%, inclusive. In some embodiments, the water content ranges from at least 40%-50%, inclusive. In some embodiments, the water content ranges from at least 40%-49%, inclusive. In some embodiments, the water content ranges from at least 40%-48%, inclusive. In some embodiments, the water content ranges from at least 40%-47%, inclusive. In some embodiments, the water content ranges from at least 40%-46%, inclusive. In some embodiments, the water content ranges from at least 40%-45%, inclusive. In some embodiments, the water content ranges from at least 40%-44%, inclusive. In some embodiments, the water content ranges from at least 40%-43%, inclusive. In some embodiments, the water content ranges from at least 40%-42%, inclusive. In some embodiments, the water content ranges from at least 40%-41%, inclusive.

In some embodiments, the water content of the hydrogel composition ranges from at least 78%-91%, inclusive. In some embodiments, the water content ranges from at least 78%-90% inclusive. In some embodiments, the water content ranges from at least 78%-89% inclusive. In some embodiments, the water content ranges from at least 78%-88% inclusive. In some embodiments, the water content ranges from at least 78%-87% inclusive. In some embodiments, the water content ranges from at least 78%-86% inclusive. In some embodiments, the water content ranges from at least 78%-85% inclusive. In some embodiments, the water content ranges from at least 78%-84% inclusive. In some embodiments, the water content ranges from at least 78%-83%. inclusive In some embodiments, the water content ranges from at least 78%-82% inclusive. In some embodiments, the water content ranges from at least 78%-81% inclusive. In some embodiments, the water content ranges from at least 78%-80% inclusive. In some embodiments, the water content ranges from at least 78%-79% inclusive. In some embodiments, the water content ranges from at least 79%-92% inclusive. In some embodiments, the water content ranges from at least 80%-92% inclusive. In some embodiments, the water content ranges from at least 81%-92% inclusive. In some embodiments, the water content ranges from at least 82%-92% inclusive. In some embodiments, the water content ranges from at least 83%-92% inclusive. In some embodiments, the water content ranges from at least 84%-92% inclusive. In some embodiments, the water content ranges from at least 85%-92% inclusive. In some embodiments, the water content ranges from at least 86%-92% inclusive. In some embodiments, the water content ranges from at least 87%-92% inclusive. In some embodiments, the water content ranges from at least 88%-92% inclusive. In some embodiments, the water content ranges from at least 89%-92% inclusive. In some embodiments, the water content ranges from at least 90%-92% inclusive. In some embodiments, the water content ranges from at least 91%-92% inclusive. In some embodiments, the water content ranges from at least 80%-88% inclusive. In some embodiments, the water content ranges from at least 82%-84% inclusive. In some embodiments, the water content ranges from at least 45%-85%, inclusive. In some embodiments, the water content ranges from at least 50%-80%, inclusive. In some embodiments, the water content ranges from at least 55%-75%, inclusive. In some embodiments, the water content ranges from at least 60%-70%, inclusive.

In some embodiments, the water content of the hydrogel composition ranges from at least 41%-92%, inclusive. In some embodiments, the water content ranges from at least 42%-92%, inclusive. In some embodiments, the water content ranges from at least 43%-92%, inclusive. In some embodiments, the water content ranges from at least 44%-92%, inclusive. In some embodiments, the water content ranges from at least 45%-92%, inclusive. In some embodiments, the water content ranges from at least 46%-92%, inclusive. In some embodiments, the water content ranges from at least 47%-92%, inclusive. In some embodiments, the water content ranges from at least 48%-92%, inclusive. In some embodiments, the water content ranges from at least 49%-92%, inclusive. In some embodiments, the water content ranges from at least 50%-92%, inclusive. In some embodiments, the water content ranges from at least 51%-92%, inclusive. In some embodiments, the water content ranges from at least 52%-92%, inclusive. In some embodiments, the water content ranges from at least 53%-92%, inclusive. In some embodiments, the water content ranges from at least 54%-92%, inclusive. In some embodiments, the water content ranges from at least 55%-92%, inclusive. In some embodiments, the water content ranges from at least 56%-92%, inclusive. In some embodiments, the water content ranges from at least 57%-92%, inclusive. In some embodiments, the water content ranges from at least 58%-92%, inclusive. In some embodiments, the water content ranges from at least 59%-92%, inclusive. In some embodiments, the water content ranges from at least 60%-92%, inclusive. In some embodiments, the water content ranges from at least 61%-92%, inclusive. In some embodiments, the water content ranges from at least 62%-92%, inclusive. In some embodiments, the water content ranges from at least 63%-92%, inclusive. In some embodiments, the water content ranges from at least 64%-92%, inclusive. In some embodiments, the water content ranges from at least 65%-92%, inclusive. In some embodiments, the water content ranges from at least 66%-92%, inclusive. In some embodiments, the water content ranges from at least 67%-92%, inclusive. In some embodiments, the water content ranges from at least 68%-92%, inclusive. In some embodiments, the water content ranges from at least 69%-92%, inclusive. In some embodiments, the water content ranges from at least 70%-92%, inclusive. In some embodiments, the water content ranges from at least 71%-92%, inclusive. In some embodiments, the water content ranges from at least 72%-92%, inclusive. In some embodiments, the water content ranges from at least 73%-92%, inclusive. In some embodiments, the water content ranges from at least 74%-92%, inclusive. In some embodiments, the water content ranges from at least 75%-92%, inclusive. In some embodiments, the water content ranges from at least 76%-92%, inclusive. In some embodiments, the water content ranges from at least 77%-92%, inclusive. In some embodiments, the water content ranges from at least 78%-92%, inclusive. In some embodiments, the water content ranges from at least 79%-92%, inclusive. In some embodiments, the water content ranges from at least 80%-92%, inclusive. In some embodiments, the water content ranges from at least 81%-92%, inclusive. In some embodiments, the water content ranges from at least 82%-92%, inclusive. In some embodiments, the water content ranges from at least 83%-92%, inclusive. In some embodiments, the water content ranges from at least 84%-92%, inclusive. In some embodiments, the water content ranges from at least 85%-92%, inclusive. In some embodiments, the water content ranges from at least 86%-92%, inclusive. In some embodiments, the water content ranges from at least 87%-92%, inclusive. In some embodiments, the water content ranges from at least 88%-92%, inclusive. In some embodiments, the water content ranges from at least 89%-92%, inclusive. In some embodiments, the water content ranges from at least 90%-92%, inclusive. In some embodiments, the water content ranges from at least 91%-92%, inclusive.

The water content of the hydrogel composition used to fabricate the medical device, such as the exemplary scaffolds, can allow for robustness and ease of handling of the scaffold. For example, a water content that is too high (e.g., above 92% w/w) can create more flexibility in the scaffold or implant, resulting in potentially greater difficulty for handling and damage to the scaffold or implant. A water content ranging between 78% and 92%, inclusive, can provide a pliable yet sufficiently strong/stiff material that can be easily handled during manufacturing and surgery. For dental tissue regeneration, the hydrogel composition water content range can be between 40% and 92%, inclusive. The semi-synthetic composition of the scaffold or implant (e.g., synthetic and collagen) further assists with biocompatibility.

Injectable scaffolds are appealing options for gum disease treatment as such scaffolds can minimize the risk and complications associated with surgical implantations. In addition, cells (such as MSCs, and/or fibroblasts, [see Soundararaj an, M. and Kannan, S. “Fibroblasts and mesenchymal stem cells: two sides of the same coin? J. Cell Physiol. (2018) 233 (12): 9099-9109]) can be mixed easily with injectable scaffolds, resulting in a homogeneous distribution of the cells. Injectable scaffolds are gel-like and can be directly injected into cavities of various shapes and sizes in a minimally invasive manner. Because of their gel-like nature, these materials are typically weak and have been used in applications that necessitate less stringent physical demands. To overcome this weakness, traditional injectable scaffolds may be designed to solidify in situ once injected, allowing the scaffold to form a three-dimensional template with desired mechanical properties on which cells can adhere.

Collagen and collagen-hybrid injectable scaffolds have been used to demonstrate their efficacy in treating various gum diseases in different in vivo studies with variable success. For example, collagen hydrogel injected in the root canal of nude mice for pulp and dentin regeneration failed to show any efficacy as the hydrogen contracted during transplantation in the root canal [See Regen. Med. (2009) 4:697-707]. The mechanical properties of the injectable scaffold made solely of collagen did not meet expectations. See id. On the other hand, injectable scaffolds made from a combination of collagen and other polymers have shown improved mechanical properties. As an example, PEG-fibrous collagen injectable scaffolds have been found to withstand higher compressive load [See Ann. Biomed. Eng. (2015) 43: 2618-2629] while hydroxyapatite-collagen-alginate hydrogels used for osteochondral regeneration showed enhanced tensile strength, better compressive modulus and better cell viability [See Biomed. Mater. (2014) 9:065004].

Gum disease studies using traditional collagen or collagen hybrid scaffolds have generally been performed using preformed scaffolds. These scaffolds are designed to have a predetermined shape and are intended to use in areas where mechanical strength is required. Because these scaffolds are implanted, there is a higher risk of complications associated with surgery. Incorporating cells into these scaffolds is also challenging as obtaining a uniform distribution of the cells in the preform is a problem. Nevertheless, such preformed scaffolds are desirable because of their improved mechanical properties, and have been used in in vitro, in vivo and clinical studies. For example, collagen-chitosan-glycerol preformed scaffolds have been used in vitro as an alternative treatment for gingival recession [See J. Int. Dent. Med. Res. (2017) 10:118-122 and J. Biomim. Biomater. Biomed. Eng. (2019) 40:101-108]. These materials met criteria for gingival recession application due to their improved physical properties. Preformed collagen scaffolds were implanted in beagle dogs for the treatment of gingival tissue regeneration [See Oral Surg. Oral Med. Oral Pathol. Oral Radiol. (2017) 124: 248-354]. Scaffolds with a pH of 7.4 were suited for gingival regeneration. In a 3 to 12 months clinical trial study involving 15 patients [See Braz. Oral Res. (2019) 33], a preformed collagen matrix was found to be comparable to a connective tissue graft in promoting healing in gingival recession.

In some embodiments, the first synthetic polymer is 2-methacryloyloxyethyl phosphorylcholine (MPC) and the second synthetic polymer is poly(ethylene glycol)diacrylate (PEGDA). In some embodiments, length of the PEGDA polymer is between 200 and 700 Da, inclusive, i.e., about 200 Da, about 210, about 220, about 230, about 240, about 250, about 260, about 270, about 280 about 290 Da, about 300 Da, about 310, about 320, about 330, about 340, about 350, about 360, about 370, about 380, about 390 Da, about 400 Da, about 410, about 420, about 430, about 440, about 450, about 460, about 470, about 480, about 490 Da, about 500 Da, about 510, about 520, about 530, about 540, about 550, about 560, about 570, about 580, about 590 Da, about 600 Da, about 610, about 620, about 630, about 640, about 650, about 660, about 670, about 680, about 690 Da or about 700 Da. In some embodiments, length of the PEGDA polymer is greater than about 700 Da.

In some embodiments, initiation of polymerization and cross-linking of the two synthetic polymers is by an ultraviolet light initiator (at a wavelength of about, e.g., 360-405 nm, inclusive, 360-400 nm inclusive, 360-390 nm inclusive, 360-380 nm inclusive, 360-370 nm inclusive, 370-405 nm inclusive, 380-405 nm inclusive, 390-405 nm inclusive, 400-405 nm inclusive, 360 nm inclusive, 370 nm inclusive, 380 nm inclusive, 390 nm inclusive, 400 nm inclusive, 405 nm inclusive, or the like). In some embodiments, the cross-linking agent is PEGDA. In some embodiments, the cross-linking agent can be any multi-arm PEG acrylate or methacrylate (i.e., 3 or 4 or 8 arm PEG acrylate or methacrylate). In some embodiments, time available for completion of polymerization and cross-linking is between 5 and 30 minutes, inclusive. In some embodiments of the composition, the weight ratio of collagen: PEGDA in the composition is about 4:1. In some embodiments, the weight ratio of PEGDA/MPC ranges from 1:3 to about 1:1, inclusive, i.e., at least 1:3, at least 1:2, or at least 1:1. In some embodiments, when the weight ratio of collagen: PEGDA in the composition is about 4:1 and weight ratio of PEGDA/MPC ranges from 1:3 to about 1:1 (i.e., 1:3, 1:2, or 1:1); water content of the IPN ranges from 90% to about 96% inclusive;

In some embodiments, the weight ratio of collagen:PEGDA ranges from about 1:3 to about 1:10, inclusive; i.e., at least 1:3, at least 1:4, at least 1:5, at least 1:6, at least 1:7, at least 1:8, at least 1:9 or at least 1:10. In some embodiments, weight ratio of PEGDA/MPC ranges from about, e.g., 1:0.5-0.5:1, 1:0.6-0.5:1, 1:0.7-0.5:1, 1:0.8-0.5:1, 1:0.9-0.5:1, 1:1-0.5:1, 1:0.5-0.6-:, 1:0.5-0.7:1, 1:0.5-0.8:1, 1:0.5-0.9:1, 1:0.5-1:1, or the like. In some embodiments, when the weight ratio of collagen: PEGDA ranges from about 1:3 to about 1:10, inclusive; and the weight ratio of PEGDA/MPC ranges from 1:0.5 to 0.05:1, inclusive, water content of the IPN ranges from about 78% to about 92%, inclusive. In some embodiments, for injectable hydrogels, ultraviolet light can be used for crosslinking, as the live cell population(s) would be added to the hydrogel composition only after the gel is formed. For hard implants or scaffolds where the cell population(s) may be mixed with the implant precursor before crosslinking, a different crosslinking chemistry (e.g., EDC chemistry) may be used.

The hydrogel composition of the present disclosure includes a combination of elements that assist with achieving the discussed biocompatibility and improved handling. In some embodiments, the percentage by weight of the collagen within the hydrogel composition can be about, e.g., 1%-5%, inclusive 1-4% inclusive, 1-3% inclusive, 1-2%, inclusive 2-5% inclusive, 3-5%, inclusive 4-5% inclusive, 2-4% inclusive, 3-4% inclusive, 1%, 1.1%, 1.2%, 1.3%, 1.4%, 1.5%, 1.6%, 1.7%, 1.8%, 1.9, 2%, 2.1%, 2.2%, 2.3%, 2.4% 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, or the like. In some embodiments, the percentage by weight of the synthetic polymer within the hydrogel composition can be about, e.g., 1.5-7.2%, inclusive, 1.5-7% inclusive, 1.5-6% inclusive, 1.5-5% inclusive, 1.5-4% inclusive, 1.5-3% inclusive, 1.5-2% inclusive, 2-7.2% inclusive, 3-7.2% inclusive, 4-7.2% inclusive, 5-7.2% inclusive, 6-7.2% inclusive, 7-7.2% inclusive, 1.5-7%, inclusive 2-7% inclusive, 3-7% inclusive, 4-7% inclusive, 5-7% inclusive, 6-7% inclusive, 1.5%, 1.6%, 1.7%, 1.8%, 1.9%, 2%, 2.1%, 2.2%, 2.3%, 2.4% 2.5%, 2.6%, 2.7%, 2.8%, 2.9%, 3%, 3.1%, 3.2%, 3.3%, 3.4%, 3.5%, 3.6%, 3.7%, 3.8%, 3.9% 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, or the like. In some embodiments, the collagen can be a porcine atelocollagen, type 1, obtained from Nippi Collagen of North America Inc. However, it should be understood that any similar collagen can be used.

Polymers used to prepare the described hydrogel composition can be any biocompatible polymer or polymer combination that achieves the desired properties, i.e., moldability, biocompatibility, hydrophilicity, and permeability.

Exemplary biocompatible biodegradable polymers include, without limitation, a poly(lactide); a poly(glycolide); a poly(lactide-co-glycolide); a poly(lactic acid); a poly(glycolic acid); a poly(lactic acid-co-glycolic acid); a poly(caprolactone); a poly(orthoester); a polyanhydride; a poly(phosphazene); a polyhydroxyalkanoate; a poly(hydroxybutyrate); a polycarbonate; a tyrosine polycarbonate; a polyamide; a polyesteramide; a polyester; a poly(dioxanone); a poly(alkylene alkylate); a polyether (such as polyethylene glycol, PEG, and polyethylene oxide, PEO); polyvinyl pyrrolidone or PVP; a polyurethane; a polyetherester; a polyacetal; a polycyanoacrylate; a poly(oxyethylene)/poly(oxypropylene) copolymer; a polyacetal, a polyketal; a polyphosphate; a (phosphorous-containing) polymer; a polyphosphoester; a polyhydroxyvalerate; a polyalkylene oxalate; a polyalkylene succinate; or a poly(maleic acid). The water-soluble, biocompatible polymer poly(2-methacryloyloxyethyl phosphorylcholine) (PMPC) is a zwitterionic polymer that is able to form a more compact conformation in aqueous solution than poly(ethylene glycol) (PEG).

Exemplary non-degradable biocompatible polymers include, without limitation, polysiloxane, polyvinyl alcohol, polyimide a polyacrylate; a polymer of ethylene-vinyl acetate, EVA; cellulose acetate; an acyl-substituted cellulose acetate; a non-degradable polyurethane; a polystyrene; a polyvinyl chloride; a polyvinyl fluoride; a poly(vinyl imidazole); a silicone-based polymer (for example, Silastic® and the like), a chlorosulphonate polyolefin; a polyethylene oxide; polysiloxane, polyvinyl alcohol, and polyimide, or a blend or copolymer thereof.

Exemplary copolymers may include, hydroxyethyl methacrylate and methyl methacrylate, and hydroxyethyl methacrylate copolymerized with polyvinyl pyrrolidone (PVP, to increase water retention) or ethylene glycol dimethacrylic acid (EGDM). Nexofilcon A (Bausch & Lomb) is a hydrophilic copolymer of 2-hydroxyethyl methacrylate and N-vinyl pyrrolidone.

Exemplary block polymers comprising blocks of hydrophilic biocompatible polymers or biopolymers or biodegradable polymers may include polyethers, including polyethylene glycol, PEG; polyethylene oxide, PEO; polypropylene oxide, PPO, perfluoropolyethers (PFPEs) and block copolymers comprised of combinations thereof.

In some embodiments, the hydrophilic polymer comprises a hydrogel polymer. Hydrogels generally comprise a variety of polymers. Exemplary polymers include acrylic acid, acrylamide and 2-hydroxyethylmethacrylate (HEMA). For example, cross-linked poly (acrylic acid) of high molecular weight is commercially available as Carbopol® (B.F. Goodrich Chemical Co., Cleveland, Ohio). Polyethylene glycol diacrylate (PEGDA 400) is a long-chain, hydrophilic, crosslinking monomer. Methacryloyloxyethyl phosphorylcholine (MPC), containing a phosphorylcholine group in the side chain, is a monomer to mimic the phospholipid polar groups contained with cell membranes. Polyoxamers, commercially available as Pluronic® (BASF-Wyandotte, USA), are thermal setting polymers formed by a central hydrophobic part (polyoxypropylene) surrounded by a hydrophilic part (ethylene oxide). (4-(4,6-dimethoxy-1,3,5-triazin-2-yyl)-4methylmorpholinium chloride (DMTMM) or N-3-dimethylaminopropyl)-N′-ethylcarbodiimide hydrochloride and N-hydroxysuccinimide (EDC/NHS) may be useful to synthesize hyaluronan derivatives. (See, D'Este, M. et al., Carbohydrate Polymers (2014) 108: 239-246). Cellulosic derivatives most commonly used in ophthalmology include: methylcellulose; hydroxyethylcellulose (HEC), hydroxypropylcellulose (HPC), hydroxypropylmethylcellulose (HPMC) and sodium carboxymethylcellulose (CMC Na). Photocrosslinked poly(ethylene glycol) diacrylate (PEGDA) hydrogels displaying collagen mimetic peptides (CMPs) that can be further conjugated to bioactive molecules via CMP-CMP triple helix association are described in Stahl, P J et al. Soft Matter (2012) 8: 10409-10418.

In some embodiments, a first polymer and a second polymer comprise one or more different non-repeating units, such as, for example, an end group, or a non-repeating unit in the backbone of the polymer. In some embodiments, the first polymer and the second polymer comprise one or more different end groups. For example, the first polymer can have a more polar end group than one or more end group(s) of the second polymer. According to some such embodiments, the first polymer will be more hydrophilic, relative to a second polymer (with the less polar end group) alone. According to some such embodiments, the first polymer comprises one or more carboxylic acid end groups, and the second polymer comprises one or more ester end groups.

In some embodiments, the hydrogel composition material comprises a polymer matrix. The polymer matrix does not necessarily, but can, comprise cross-linked or intertwined polymer chains. In some embodiments, portions of the polymer matrix can comprise only one of the first and second synthetic polymer.

According to some embodiments, the polymer composition is biocompatible and nontoxic.

According to another aspect, the hydrogel composition comprising the polymer matrix is flexible and can be configured as any of a variety of forms, types or designs that are described, used or practiced in the art. For example, the hydrogel composition material comprising a polymer matrix can be configured into the physical form of a film, a fiber, a filament, a sheet, a thread, a cylindrical implant, a asymmetrically-shaped implant or a fibrous mesh. In some embodiments, the hydrogel composition material is formed into the physical form of a film, meaning a thin skin or membrane. In some embodiments, the hydrogel composition material is formed into the physical form of a fiber, meaning a threadlike part. In some embodiments, the hydrogel composition material is formed into the physical form of a filament, meaning a fine, threadlike fiber. In some embodiments, the hydrogel composition material is formed into the physical form of a sheet, meaning a broad thin continuous piece of the hydrogel material. In some embodiments, the hydrogel composition material is formed into the physical form of a thread, meaning a fine cord composed of a strand or multiple strands. In some embodiments, the hydrogel composition material is formed into the physical form of a fibrous mesh. In some embodiments, the fibrous mesh has the form of a woven or non-woven material. In some embodiments, the fibrous mesh has the form of a felt, meaning a material comprising fibers that are not woven together but instead matted or wrought into a compact substance. In some embodiments, the fibrous mesh has the form of a gauze, meaning a thin, light loosely woven material. In some embodiments, the fibrous mesh has the form of a sponge, meaning a porous material with characteristic compressibility that can absorb many times its own weight in water.

In some embodiments, the hydrogel composition material formed into the physical form of a film, a fiber, a filament, a sheet, a thread, a cylindrical implant, a asymmetrically-shaped implant or a fibrous mesh is fabricated from a hydrogel composition having a water content ranging from, e.g., 50% to 92% (w/w inclusive), 78% to 92% (w/w) inclusive, or the like. The physical form formed from the hydrogel having the water content ranging from 50% to 92% (w/w inclusive), 78% to 92% (w/w) inclusive, or the like, provides for improved handling of the physical form and biocompatibility of the physical form with patient tissue. In some embodiments, the hydrogels can be synthesized using a combination of biopolymers and synthetic monomers and/or polymers. Such a hydrogel-based form is expected to be biocompatible.

In some embodiments, the collagen-hydrogel polymer is loaded with cells after polymerization and crosslinking of the collagen-MPC-PEGDA polymer matrix is complete. In some embodiments, the polymer comprises a channel forming agent or porogen, e.g., CaCl2. In some such embodiments, porous scaffolds for post-polymerization loading of cells may comprise porogen leaching to establish good interconnectivity in the pore structure, which is important for cell seeding and cell migration throughout the scaffolds, while maintaining mechanical strength of the polymer. [Chen, G. and Kawazoe, N. Preparation of polymer-based porous scaffolds for tissue engineering, Chapter 5 in Characterization and design of tissue scaffolds, Paul Tomlins, Ed. Elsevier Ltd. (2016) pp. 105-124].

In some embodiments, the porogen comprises ice particulates. In some embodiments, the porogen comprises sodium chloride. In some embodiments, the porogen comprises a combination of ice particulates and sodium chloride. A method using ice particulates as a porogen material that can control pore structure and also increase pore interconnectivity of porous scaffolds has been described [Id., citing Zhang, Q. et al. Preparation of collagen porous scaffolds with a gradient pore size structure using ice particulates. Mater. Lett. (2013) 107: 280-83]. In this method, ice particulates are prepared by spraying pure water in liquid nitrogen The ice particulates are sieved to obtain ice particulates with diameters in a specific range. The diameter range can be selected such that the scaffold pore size provides maximum efficiency of the implant. In some embodiments, the diameter of the pore size can be between 50-600 microns, inclusive. In some embodiments, the diameter of the pore size can be between 75-600 microns, inclusive. In some embodiments, the diameter of the pore size can be between 100-600 microns, inclusive. In some embodiments, the diameter of the pore size can be between 125-600 microns, inclusive. In some embodiments, the diameter of the pore size can be between 150-600 microns, inclusive. In some embodiments, the diameter of the pore size can be between 175-600 microns, inclusive. In some embodiments, the diameter of the pore size can be between 200-600 microns, inclusive. In some embodiments, the diameter of the pore size can be between 225-600 microns, inclusive. In some embodiments, the diameter of the pore size can be between 250-600 microns, inclusive. In some embodiments, the diameter of the pore size can be between 275-600 microns, inclusive. In some embodiments, the diameter of the pore size can be between 300-600 microns, inclusive. In some embodiments, the diameter of the pore size can be between 325-600 microns, inclusive. In some embodiments, the diameter of the pore size can be between 350-600 microns, inclusive. In some embodiments, the diameter of the pore size can be between 375-600 microns, inclusive. In some embodiments, the diameter of the pore size can be between 400-600 microns, inclusive. In some embodiments, the diameter of the pore size can be between 425-600 microns, inclusive. In some embodiments, the diameter of the pore size can be between 450-600 microns, inclusive. In some embodiments, the diameter of the pore size can be between 475-600 microns, inclusive. In some embodiments, the diameter of the pore size can be between 500-600 microns, inclusive. In some embodiments, the diameter of the pore size can be between 525-600 microns, inclusive. In some embodiments, the diameter of the pore size can be between 550-600 microns, inclusive. In some embodiments, the diameter of the pore size can be between 575-600 microns, inclusive. In some embodiments, the diameter of the pore size can be between 50-575 microns, inclusive. In some embodiments, the diameter of the pore size can be between 50-550 microns, inclusive. In some embodiments, the diameter of the pore size can be between 50-525 microns, inclusive. In some embodiments, the diameter of the pore size can be between 50-500 microns, inclusive. In some embodiments, the diameter of the pore size can be between 50-475 microns, inclusive. In some embodiments, the diameter of the pore size can be between 50-450 microns, inclusive. In some embodiments, the diameter of the pore size can be between 50-425 microns, inclusive. In some embodiments, the diameter of the pore size can be between 50-400 microns, inclusive. In some embodiments, the diameter of the pore size can be between 50-375 microns, inclusive. In some embodiments, the diameter of the pore size can be between 50-350 microns, inclusive. In some embodiments, the diameter of the pore size can be between 50-325 microns, inclusive. In some embodiments, the diameter of the pore size can be between 50-300 microns, inclusive. In some embodiments, the diameter of the pore size can be between 50-275 microns, inclusive. In some embodiments, the diameter of the pore size can be between 50-250 microns, inclusive. In some embodiments, the diameter of the pore size can be between 50-225 microns, inclusive. In some embodiments, the diameter of the pore size can be between 50-200 microns, inclusive. In some embodiments, the diameter of the pore size can be between 50-175 microns, inclusive. In some embodiments, the diameter of the pore size can be between 50-150 microns, inclusive. In some embodiments, the diameter of the pore size can be between 50-125 microns, inclusive. In some embodiments, the diameter of the pore size can be between 50-100 microns, inclusive. In some embodiments, the diameter of the pore size can be between 50-75 microns, inclusive. In some embodiments, the diameter of the pore size can be between 100-500 microns, inclusive. In some embodiments, the diameter of the pore size can be between 200-300 microns, inclusive. In some embodiments, the diameter of the pore size can be between 100-400 microns, inclusive. In some embodiments, the diameter of the pore size can be between 100-300 microns, inclusive. In some embodiments, the diameter of the pore size can be between 200-500 microns, inclusive. In some embodiments, the diameter of the pore size can be between 300-500 microns, inclusive. In some embodiments, the diameter of the pore size can be between 400-500 microns, inclusive.

The ice particulates are then mixed with aqueous solution of polymers after the temperature is balanced at a point at which the ice particulates remain frozen whereas the surrounding aqueous solution does not freeze. The mixture is then frozen at a low temperature and freeze-dried. By controlling the freezing temperature during the freezing process, the ice particulates work as nuclei to initiate the formation of new ice crystals, which should be connected with the nuclear ice particulates. Removal of the ice particulates and the newly formed ice crystals results in formation of highly interconnected pore structures in the porous scaffolds. Collagen porous scaffolds prepared by this method have interconnected pore structures with large pore surrounded with small pores. Collagen scaffolds prepared with 50% ice particulates show the most homogeneous pore structures. When 25% of ice particulates is used, there is some distance between the large pores. When the ratio of ice particulates is high, the collagen aqueous solution filling the spaces between the spherical ice particulates decreases and the collagen matrix surrounding the large pores decreases. In addition, mixing the ice particulates and the collagen aqueous solution becomes difficult when the concentration of ice particulates is too high. In some embodiments, the scaffolds are cross-linked after freeze drying.

In some embodiments, the collagen concentration affects the pore structures. For example, when collagen scaffolds are prepared at a 50% (w/v) ice particulate ratio and 1%, 2%, and 3% (w/v) collagen aqueous solution, the scaffolds have different pore structures. In some embodiments, the collagen scaffold prepared with 2% collagen solution and an ice particulate/collagen solution ratio of 50% shows the most homogeneous pore structure. In some embodiments, when the collagen concentration is fixed at 2% and the ratio of ice particulates is changed, the Young's modulus of the collagen porous scaffolds increases in the following order: 75%<25%<50%. In some embodiments, the collagen scaffolds prepared with 50% ice particulates have the highest Young's modulus.

By using ice particulates as a porogen, collagen porous scaffolds with a gradient pore size structure have been prepared that have an interconnected pore structure. When such scaffolds are used for culture of bovine articular chondrocytes, the cells adhere and are homogeneously distributed throughout the scaffolds. [Id., citing Zhang, Q. et al. Preparation of collagen scaffolds with controlled pore structures and improved mechanical property for cartilage tissue engineering. J. Bioact. Compat. Polym. (2013) 28: 426-38].

In some embodiments, the ice particulate method can be used to prepare porous structures with fully open surface pores to facilitate cell seeding. [Id., citing Ko, Y G et al. Preparation of collagen-glycosaminoglycan sponges with open surface porous structures using ice particulate template method. Macromol. Biosci. (2010) 10: 860-71; Ko, Y G et al; Preparation of novel collagen sponges using an ice particulate template. J. Bioac ct. Compat. Polym. (2010) 25: 360-73]. In some embodiments, the ice particulates are embossed on a substrate and used to control the surface pore structures of scaffolds as required for smooth cell seeding and uniform cell distribution. In some embodiments, the ice particulate template can be prepared by freezing or injecting micrometer-sized water droplets on a substrate.

In some embodiments, three-dimensional porous scaffolds with micropatterned pores can be prepared by using micropatterned ice particulates or ice lines as a template [Id., citing Oh, H H et al. Preparation of porous collagen scaffolds with micropatterned structures. Adv. Mater. (2012) 24: 4311-16).

In some embodiments, the open surface pores may subsequently be sealed prior to implantation. According to some embodiments, the open surface pores may be sealed by coating the polymer with collagen.

In some embodiments, the thickness of the polymer scaffold formed from the hydrogel composition will be appropriate to maintain survival of the population of cells embedded in the polymer scaffold. For example, the polymer can be populated with an appropriate cell type and can be used as a transportation system in gel form. In such instances, the polymer acts as a means for stem cell mediated healing. Since in vivo, most cells exist within about 100 μm of a capillary, and diffusion is usually adequate for cell and tissue survival over this distance [Drury and Moody, Biomaterials (2003) 24: 4337-51], the thickness of the polymer scaffold in some embodiments may range from, e.g., 0.400-0.700 mm inclusive, 0.410-0.700 mm inclusive, 0.420-0.700 mm inclusive, 0.430-0.700 mm inclusive, 0.440-0.700 mm inclusive, 0.450-0.700 mm inclusive, 0.460-0.700 mm inclusive, 0.470-0.700 mm inclusive, 0.480-0.700 mm inclusive, 0.490-0.700 mm inclusive, 0.500-0.700 mm inclusive, 0.510-0.700 mm inclusive, 0.520-0.700 mm inclusive, 0.530-0.700 mm inclusive, 0.540-0.700 mm inclusive, 0.550-0.700 mm inclusive, 0.560-0.700 mm inclusive, 0.570-0.700 mm inclusive, 0.580-0.700 mm inclusive, 0.590-0.700 mm inclusive, 0.600-0.700 mm inclusive, 0.610-0.700 mm inclusive, 0.620-0.700 mm inclusive, 0.630-0.700 mm inclusive, 0.640-0.700 mm inclusive, 0.650-0.700 mm inclusive, 0.660-0.700 mm inclusive, 0.670-0.700 mm inclusive, 0.680-0.700 mm inclusive, 0.690-0.700 mm inclusive, 0.400-0.690 mm inclusive, 0.400-0.680 mm inclusive, 0.400-0.670 mm inclusive, 0.400-0.660 mm inclusive, 0.400-0.650 mm inclusive, 0.400-0.640 mm inclusive, 0.400-0.630 mm inclusive, 0.400-0.620 mm inclusive, 0.400-0.610 mm inclusive, 0.400-0.600 mm inclusive, 0.400-0.590 mm inclusive, 0.400-0.580 mm inclusive, 0.400-0.570 mm inclusive, 0.400-0.560 mm inclusive, 0.400-0.550 mm inclusive, 0.400-0.540 mm inclusive, 0.400-0.530 mm inclusive, 0.400-0.520 mm inclusive, 0.400-0.510 mm inclusive, 0.400-0.500 mm inclusive, 0.400-0.490 mm inclusive, 0.400-0.480 mm inclusive, 0.400-0.470 mm inclusive, 0.400-0.460 mm inclusive, 0.400-0.450 mm inclusive, 0.400-0.440 mm inclusive, 0.400-0.430 mm inclusive, 0.400-0.420 mm inclusive, 0.400-0.410 mm inclusive, 0.450-0.650 mm inclusive, 0.500-0.600 mm inclusive, or the like.

In some embodiments, a population of cells that has been well-cultivated in an appropriate medium (e.g., DMEM), supplemented with various nutritional and growth factors, is washed and suspended in a polymer solution at the desired cell density (e.g., 0.5×10⁵, 0.6×10⁵, 0.7×10⁵, 0.8×10⁵, 0.9×10⁵, 1×10⁵, 2×10⁵, 3×10⁵, 4×10⁵, 5×10⁵, 6×10⁵, 7×10⁵, 8×10⁵, 9×10⁵, or 10×10⁵ cells per mL) as described by Zhang, R. et al. Biomaterials Sci. (2019) 7: 2973. The hydrogel containing the embedded cells is maintained at 37° C. in a humidified atmosphere containing 5% CO₂ for several days (e.g., 5 days). After the 5 day culture, the hydrogel construct comprising the immobilized cells is collected and washed with DMEM twice and then with PBS once before implantation. In some embodiments, re-collection of the immobilized cells in the hydrogel can be performed by solubilization of the hydrogel; the released cells can be washed by centrifugation and resuspended for counting and live/dead staining.

In some embodiments, the population of live cells is a population of MSCs. In some embodiments, the MSCs are obtained from a human subject. In some embodiments, the human subject is a healthy human subject. In some embodiments, identity of the MSCs is confirmed by a biomarker signature comprising CD29, CD44, and CD105. In some embodiments, the MSCs are derived from adipose tissue, dental tissue or whole blood. In some embodiments, the dental tissue includes, without limitation, craniofacial bone, dental pulp, PDL, a dental follicle, tooth germ, apical papilla, oral mucosa, gingival tissue and periosteum (see, e.g., Chalissery, E P et al. (2017) J. Tissue Engineering 8: 1-17) of the normal healthy subjects. In some embodiments, the normal healthy subjects are aged 18-34 years old.

Conventional enzymatic methods, using enzymes such as collagenase, trypsin, or dispase, are widely used to isolate MSCs from adipose tissue. Although the isolation techniques for adipose tissue-derived cells are rather diverse, they follow a certain standard procedure. Differences lie mainly in numbers of washing steps, enzyme concentrations, centrifugation parameters, erythrocyte lysis methods as well as in filtration, and eventually culture conditions [Oberbauer E, et al., Cell Regen (Lond). 2015; 4: 7, citing Zuk P A, et al., Mol Biol Cell. 2002; 13(12): 4279-95; Gimble J, Guilak F. Cytotherapy. 2003; 5(5): 362-9; Carvalho P P, et al., Tissue Eng Part C Meth. 2013; 19(6): 473-8]. An exemplary protocol for isolating MSCs from adipose tissue includes the steps of obtaining adipose tissue by surgical resection or lipoaspiration; washing the tissue 3-5 times for 5 minutes in PBS each wash, discarding the lower phase until clear; adding collagenase and incubating 1-4 hr at 37° C. on a shaker; adding 10% FBS to neutralize the collagenase; centrifuging the digested fat at 800×g for 10 min; aspirating floating adipocytes, lipids and liquid, leaving the stromal vascular fraction (SVF) pellet; resuspending the SVF pellet in 160 mM NH₄Cl and incubating for 10 minutes at room temperature; centrifuging at 400×g for 10 min at room temperature; layering cells on a Percoll or Histopaque gradient; centrifuging at 1000×g for 30 minutes at room temperature; washing cells twice with PBS and centrifuging at 400×g for 10 min between each wash; resuspending the cell pellet in PBS and filtering cells through a 100 μM nylon mesh; passing the cells through a 400 μM nylon mesh; centrifuging at 400×g for 10 minutes; resuspending the cell pellet in 40% FBS/DMEM culture medium and plating the cells. The plastic-adherent cell fraction, including ASCs, can be obtained after passaging or cryopreservation or further cultivated for expansion for a more homogeneous ASC population (Id.).

Similar to adipose tissue, generating stem cells from dental pulp is a relatively noninvasive and noncontroversial process. Deciduous teeth may be sterilized, and the dental pulp tissue separated from the pulp chamber and root canal, revealed by cutting around the cementoenamel junction using sterilized dental burs (Tsai A I, et al., Biomed Res Int. 2017: 2851906). After separation, the dental pulp may be isolated using, for example, a barbed broach or a sharp excavator (Id.). MSCs may be isolated enzymatically or non-enzymatically as described above for adipose tissue.

In an exemplary protocol for obtaining MSCs from whole blood, a 1:1 diluted mixture of PBS and peripheral blood is gently layered in a 50 ml centrifuge tube on top of the density gradient medium (e.g., Ficol-Paque™ or Lymphoprep™), and centrifuged at 800×g for 20-30 minutes at 20° C. in a swinging-bucket rotor with the brake off. The upper layer is aspirated, leaving the mononuclear cell layer (lymphocytes, monocytes and thrombocytes) undisturbed at the interface. The mononuclear cell layer is carefully transferred into a new 50 ml centrifuge tube. Cells are washed with PBS (pH 7.2) containing 2 mM EDTA, centrifuged at 300×g for 10 min at room temperature and the supernatant discarded. For removal of platelets, the cell pellet is resuspended in 50 mL buffer and centrifuged at 200×g for 10-15 minutes at room temperature. The supernatant containing the platelets is removed. This step is repeated. The cell pellet is resuspended in DMEM, 20% FBS and 1% antibiotic-antimycotic. Cultures are maintained at 37° C. in a humidified atmosphere containing 5% CO2. Suspended cells are discarded after 5-7 days of culture and adherent cells left to grow on the flask surface. Culture medium is changed every 3 days.

In some embodiments, the population of live cells may release one or more cell products during culturing into a conditioned medium. The term “conditioned medium” (or plural, media), as used herein refers to spent culture medium harvested from cultured cells containing metabolites, growth factors, RNA and proteins released into the medium by the cultured cells. In some embodiments, the population of live cells once embedded in the polymer matrix likewise may release one or more cell products into the polymer matrix that then may diffuse from the matrix into the periodontic space and effect wound healing via a paracrine effect. In some embodiments, the cell products may include one or more growth factors, fragments or variants thereof. Exemplary growth factors include epidermal growth factor (EGF), fibroblast growth factor (FGF), insulin-like growth factor (IGF), platelet derived growth factor (PDGF), transforming growth factor beta (TGFβ), bone morphogenetic proteins (BMPs), and vascular endothelial growth factor (VEGF). Exemplary angiogenic factors secreted by MSCs include vascular endothelial growth factor (VEGF) [Id., citing Kinnaird, T. et al. Marrow-derived stromal cells express genes encoding a broad spectrum of arteriogenic cytokines and promote in vitro and in vivo arteriogenesis through paracrine mechanisms. Circ Res. (2004) 94:678-685; Rehman, J. et al, Secretion of angiogenic and antiapoptotic factors by human adipose stromal cells. Circulation. (2004) 109:1292-1298], fibroblast growth factor-2 (FGF-2), Angiopoetin-1 (Ang-1) [Id., citing Kamihata, H. et al. Implantation of bone marrow mononuclear cells into ischemic myocardium enhances collateral perfusion and regional function via side supply of angioblasts, angiogenic ligands, and cytokines. Circulation (2001) 104:1046-1052], insulin-like growth factor (IGF-1) [Id., citing Togel, F. et al. Vasculotropic, paracrine actions of infused mesenchymal stem cells are important to the recovery from acute kidney injury. Am J Physiol Renal Physiol. (2007) 292:F1626-35], hepatocyte growth factor (HGF) [Id., citing Rehman, J. et al. Marrow-derived stromal cells express genes encoding a broad spectrum of arteriogenic cytokines and promote in vitro and in vivo arteriogenesis through paracrine mechanisms. Circ Res. (2004) 94:678-685], transforming growth factor (TGF)-β, monocyte chemoattractant protein (MCP-1)[Id., citing Kwon, H M et al. Multiple paracrine factors secreted by mesenchymal stem cells contribute to angiogenesis. Vascul Pharmacol. :S1537-1891, Boomsma, R A and Greenen, DL. Mesenchymal stem cells secrete multiple cytokines that promote angiogenesis and have contrasting effects on chemotaxis and apoptosis. PLoS One. 2012; 7:e35685], interleukin (IL)-6 [Id., citing Kwon, H M et al. Multiple paracrine factors secreted by mesenchymal stem cells contribute to angiogenesis. Vascul Pharmacol. :S1537-1891, Boomsma R A, Geenen D L. Mesenchymal stem cells secrete multiple cytokines that promote angiogenesis and have contrasting effects on chemotaxis and apoptosis. PLoS One. 2012; 7:e35685] and SDF-1α [Id., citing Tang, J M et al. VEGF/SDF-1 promotes cardiac stem cell mobilization and myocardial repair in the infarcted heart. Cardiovasc Res. 91:402-411]. These paracrine factors can function to trophically assist vascular 1 (Ang-1) [Id., citing Kamihata, H. et al. Implantation of bone marrow mononuclear cells into ischemic myocardium enhances collateral perfusion and regional function via side supply of angioblasts, angiogenic ligands, and cytokines. Circulation. 2001; 104:1046-1052], insulin-like growth factor (IGF-1) [Id., citing Togel, F. et al. Vasculotropic, paracrine actions of infused mesenchymal stem cells are important to the recovery from acute kidney injury. Am J Physiol Renal Physiol. 2007; 292:F1626-35], hepatocyte growth factor (HGF) [Id., citing Rehman, J. et al. Marrow-derived stromal cells express genes encoding a broad spectrum of arteriogenic cytokines and promote in vitro and in vivo arteriogenesis through paracrine mechanisms. Circ Res. 2004; 94:678-685], transforming growth factor (TGF)-β, in monocyte chemoattractant repair and regeneration processes at sites of severe tissue ischemia or damage. In addition, MSCs also secrete several anti-apoptotic factors-(e.g., VEGF, HGF, IGF-1, staniocalcin-1, transforming growth factor (TGF-β), and granulocyte macrophage derived growth factor (GM-CSF); immunomodulatory factors (inducible nitric oxide (NO), prostaglandin E2 (PGE2), 2,3-dioxygenase, the non-classical major histocompatibility antigen HGF, TGF-β, leukemia inhibitory factor (LIF), and IL-10); factors supporting tissue stem and progenitor cell proliferation (stem cell growth factor (SCF), LIF, macrophage derived growth factor (M-CSF), stromal cell derived factor-1 (SDF-1) and Ang-1); factors inhibiting fibrosis and scarring in ischemia (HGF, FGF-2, adrenomedullin); and chemoattractants (MCP-1, the macrophage inhibiting protein (MIP-1), chemokine (CC motif) ligand 5 (CCL) 5, IL-8, and SDF-1) [see Meirelles Lda et al., Mechanisms involved in the therapeutic properties of mesenchymal stem cells. Cytokine Growth Factor Rev. (2009) 20:419-427].

In some embodiments, the hydrogel polymer matrix may be supplemented with multiple supplementary growth factors or their biologically active fragments or variants [See, e.g., Drury, J L and Mooney, DJ. Biomaterials (2003) 24: 4337-51, citing Elisseeff, J. et al. Controlled-release of IGF-1 and TGF-β1 in a photopolymerizing hydrogel for cartilage tissue engineering. J. Ortho Res. (2001) 19: 1098-1104]. In some embodiments, supplementary growth factors can be tethered to the formed hydrogel polymer matrix to promote periodontic cell migration into the hydrogel [See, e.g., Drury, J L and Mooney, DJ. Biomaterials (2003) 24: 4337-51, citing Suzuki, Y. et al. Alginate hydrogel linked with synthetic oligopeptide derived from BMP-2 allows ectopic osteoinduction in vivo. J. Biomed. Mater. Res. (2000) 50: 405-9]. In some embodiments, the therapeutic amount of the one or more growth factors is effective to increase growth factor receptor-mediated signaling, to decrease inflammation, or both in the periodontium.

With respect to fragments of a biologically active full length polypeptide growth factor, in some embodiments, suitable fragments may have a continuous series of deleted residues from the amino or the carboxy terminus, or both, in comparison to the full length protein. In some embodiments, the fragments may be characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, and substrate binding regions. In some embodiments, the fragments may be produced by peptide synthesis techniques, or by cleavage of full length BMP polypeptide. In some embodiments, the fragments may be linked at their N termini, C termini, or both their N and C termini to other polypeptide sequences, thus forming fusion proteins. In some embodiments, an included fragment also includes a polypeptide or glycopolypeptide having an amino acid sequence which is partially homologous with the amino acid sequence of the polypeptide, or a fragment thereof, as disclosed above, and which at least partially retain the biological activity of the polypeptide in the assays and treatment methods of the disclosure. In some embodiments, homologues may be 50%, 70%, 80%, 80.6%, 83%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the polypeptide or fragments thereof.

With respect to variants of a biologically active full length polypeptide growth factor, and variants of fragments, such variants at least partially retain the ability to attenuate excessive cellular proliferation in the assays and treatment methods of the disclosure. In some embodiments, variants may include deletions, insertions, inversions, repeats, and substitutions selected according to general rules known in the art so as have little effect on activity. For example, guidance concerning how to make phenotypically silent amino acid substitutions is provided in Bowie et al., Science 247: 1306-1310 (1990), incorporated by reference herein in its entirety. For example, variants can be obtained by site directed mutagenesis or alanine-scanning mutagenesis (introduction of single alanine mutations at every residue in the molecule). (Cunningham and Wells, Science 244: 1081-1085 (1989). In some embodiments, variants may also have amino acid substitutions that contain, for example, one or more non-peptide bonds (which replace the peptide bonds) in the protein or peptide sequence. In some embodiments, variants may also have substitutions that include amino acid residues other than naturally occurring L-amino acids, e.g., D-amino acids or non-naturally occurring or synthetic amino acids, e.g., B or y amino acids. In some embodiments, variants may also include crosslinking groups which impose conformational constraints on the polypeptide. In some embodiments, variants may also include glycosylations, acetylations, phosphorylations and the like. In some embodiments, variants may also include (i) substitutions with one or more non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the growth factor preparation (for example, polyethylene glycol), or to target the growth factor preparation to a specific cell type, or (iv) fusion of the polypeptide with additional amino acids or additional peptides or additional polypeptides, or (v) fusion to a marker that may be used for imaging purposes, for example, a radiolabel.

The growth factor preparations of the disclosure can be prepared in any suitable manner, including through the isolation of naturally occurring polypeptides, by recombinant techniques, by polypeptide synthesis techniques, or by a combination of these methods. The preparations may be in the form of a larger protein, such as a fusion protein. It is often advantageous to include an additional amino acid sequence which contains secretory or leader sequences, pro-sequences, sequences which aid in purification, such as multiple histidine residues, or an additional sequence for stability during recombinant production.

In some embodiments, the MSC cell products include extracellular vesicles (EVs). MSCs release a significant amount of microvesicles containing mRNA with specific multiple differentiative and functional properties, as well as selected patterns of mature micro RNAs. EV composition is determined not only by the cell type but also by the physiological state of the producer cells. [van Neil, G. et al. Shedding light on the cell biology of extracellular vesicles. Nature Revs. Molec. Cell Biol. (2018) 19: 213-228]. In some embodiments, the EVs comprise exosomes comprising cargo. In some embodiments, cargo delivered by the EVs may activate various responses and processes following its delivery and internalization in a recipient cell in the periodonium.

In some embodiments, the hydrogel composition may be supplemented by exposure to a purified and enriched population of extracellular vesicles. In some embodiments, the extracellular vesicles are derived from mesenchymal stem cells (MSCs) of a healthy subject. In some embodiments, the EVs comprise exosomes comprising a cargo. In some embodiments, (a) the exosomes comprise three or more biomarkers including CD9, CD63, CD81; (b) the exosomes comprise a total protein concentration of at least 0.05 mg; 0.06 mg; 0.07 mg; 0.08 mg; 0.09 mg; 0.1 mg; 0.2 mg, 0.3 mg, 0.4 mg, 0.5 mg, 0.6 mg, 0.7 mg, 0.8 mg, 0.9 mg, or 1.0 mg; (c) the exosome cargo comprises a one or more, two or more, three or more, four or more, or five or more of vasculoendothelial growth factor (VEGF), platelet derived growth factor (PDGF), endothelial growth factor (EGF), or tumor necrosis factor alpha (TNFα); and (e) size of the exosomes is about 90-110 nm, inclusive.

In some embodiments, the EVs are purified by one or more of: a) ultracentrifugation; b) sucrose density gradient centrifugation; c) column chromatography; d) size exclusion; or e) filtration through a device containing an affinity matrix selective towards the EVs. In some embodiments, the EVs are characterized by: sedimentation at about 100,000×g, a buoyant density in sucrose of about 1.10-1.21 g/ml, and an average diameter from about 30 nm to about 200 nm. In some embodiments, the EVs comprise microvesicles whose diameter is >200 nm. [Doyle, L M and Wang, MZ. Cells (2019) 8: 727].

Methods of Use/Treatment/Delivery

According to another aspect, the formed hydrogel composition comprising live cells embedded in the polymer matrix can be administered to subjects in need thereof. Administering as used herein includes in vivo administration, as well as administration directly to tissue ex vivo. Local delivery can be by a variety of techniques that administer the formed hydrogel composition at or near the targeted site, e.g., at the gum line. Examples of local delivery techniques include, without limitation, local delivery catheters, site specific carriers, implants, direct injection, or direct applications, such as topical application.

The term “parenteral” as used herein refers to a route of administration where the active agent enters the body without going through the stomach or “gut”, and thus does not encounter the first pass effect of the liver. Examples include, without limitation, introduction into the body by way of an injection (i.e., administration by injection).

The term “local delivery by implant” as used herein describes the surgical placement of the formed hydrogel composition comprising a polymer matrix into an affected site. The implanted matrix then may release the cell products of the embedded cells by diffusion, chemical reaction, or both.

In some embodiments, a surface of the implant comprising the formed hydrogel composition may be modified to promote its adhesion at the affected site. According to some embodiments, the surface may be modified by applying a peptide to the surface, the affected site, or both. In some embodiments, the peptide is one of amino acid sequence arginine-glycine-aspartic acid (RGD) derived from an ECM protein, including fibronectin, laminin, vitronectin and collagen; one of amino acid sequence arginine-glutamic acid-aspartic acid-valine (REDV) (derived from fibronectin); one of amino acid sequence tyrosine-isoleucine-glycine-serine-arginine (YIGSR (derived from laminin); or one of amino acid sequence isoleucine-lysine-valine-alanine-valine (IKVAV) (derived from laminin). [see, e.g., Drugy, J L and Moody, DJ. Biomaterials (2003) 4337-51].

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the hydrogel composition of the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Although the examples discussed herein are based on optical inlay devices, the examples may apply to injectable or implantable scaffolds for use in treatment of gum disease.

An inlay is an optical device that corrects the refractive error of the eye by changing the anterior corneal curvature by functioning as an intrastromal implant. Design criteria for such intracorneal materials include chemical inactivity, adequate permeability to maintain the diffusion of intracorneal fluid and metabolites, and avoidance of excessive pressure or tension on the corneal tissue [Wu, J. et al. Sci China Chem. (2014) 57 (4): 501-9]. Materials considered for intrastromal implants also should have a sufficient water content to sustain nutritional transport, an isotropic refractive index to match that of the surrounding tissue, a compliance similar to the native cornea, be optically clear and biostable (meaning resistant to hydrolytic, oxidative and enzymatic degradation) and biocompatible with the stromal tissue [Id., citing McCarey, BE. Refract. Corneal Surg. (1990) 6: 40-46; McCarey, BE. Int. Ophthalmol. Clin. (1991) 31: 87-99; McCarey, BF. Et al. Arch. Ophthalmol. (1989) 107: 724-30; McCarey, B E et al. Arch. Ophthalmol. (1990) 108: 1310-15]. Although discussed herein with respect to an inlay, it should be understood that the hydrogel composition discussed in the examples herein can be used to formulate a hydrogel polymer capable of receiving and supporting live cells and their released cell products. For example, the hydrogel composition can be used to formulate a hydrogel polymer scaffold for treatment of gum disease.

### Example 1. Exemplary Polymer Hydrogel Inlays

Exemplary inlay 1. A collagen-synthetic polymer hydrogel inlay was formed from a collagen-2-Methacryloyloxyethyl phosphorylcholine (MPC)-Poly(ethylene glycol) diacrylate (PEGDA) composition. The hydrogel composition included an IPN made of a natural polymer (e.g., collagen), and two synthetic polymers (i.e., MPC and PEGDA). The water content of the composition ranged from about 94% to about 98% (referred to herein as a “high water content composition”). In some instances the hydrogel composition had a water content of between about 90% to about 96%.

The IPN for the high water content composition had a collagen/PEGDA weight ratio of about 4:1, and a PEGDA/MPC weight ratio varying from about 1:3 to about 1:1.

The length of PEGDA was small enough to serve as both a crosslinking agent and a macro-monomer i.e., between about 200 to about 700 Da. A crosslinking agent was used to crosslink collagen, while an ultraviolet (UV) initiator was used for simultaneously initiating the polymerization and crosslinking of MPC with PEGDA as a crosslinker. Instead of using a redox initiator (as generally used with traditional compositions), the hydrogel composition was cross-linked with a UV initiator which can extend the mold fabrication time up to about 5 minutes. In contrast, with traditional redox initiators, cross-linking must occur in less than 30 seconds, minimizing the time allotted to ensuring the hydrogel is properly positioned in the mold.

Exemplary inlay 2. A second inlay was formed from a collagen-MPC-PEGDA composition. The hydrogel composition included an IPN made of a natural polymer (e.g., collagen), and two synthetic polymers (i.e., MPC and PEGDA). The composition had a water content ranging from about 78% to about 92% (referred to herein as a “low water content composition”). The IPN for the low water content composition had a collagen/PEGDA weight ratio varying from about 1:3 to about 1:10, and a PEGDA/MPC weight ratio varying from about, e.g., 1:0.5-0.5:1, 1:0.6-0.5:1, 1:0.7-0.5:1, 1:0.8-0.5:1, 1:0.9-0.5:1, 1:1-0.5:1, 1:0.5-0.6-:, 1:0.5-0.7:1, 1:0.5-0.8:1, 1:0.5-0.9:1, 1:0.5-1:1, or the like. The length of PEGDA was greater than about 700 Da. A crosslinking agent was used to crosslink collagen, while an initiator (e.g., UV) was used for simultaneously initiating polymerization and crosslinking of MPC and PEDGA.

### Example 2. Method of Making IPN Hydrogels for Corneal Inlay

The collagen and the non-collagen components of the hydrogel mixture were simultaneously crosslinked in the mold cavity. While collagen was crosslinked only via DMT-MM (4-(4,6-Dimethoxy-1,3,5-triazin-2-yl)-4-methylmorpholinium chloride), two different crosslinking chemistries can be used to polymerize/crosslink the non-collagen moieties of the hydrogel.

One crosslinking chemistry can be, e.g., DMT-MM-APS/TMEDA. DMT-MM was used to slowly crosslink collagen, while the redox pair, Ammonium Persulfate (APS)/TMEDA was used to polymerize and crosslink MPC, with PEGDA as the crosslinker. The entire process was performed at room temperature.

Another crosslinking chemistry can be, e.g., DMT-MM-LAP. DMT-MM was used to slowly crosslink collagen, while a UV initiator Lithium phenyl-2,4,6-trimethylbenzoylphosphinate (LAP) was used to polymerize and crosslink MPC, with PEGDA as the crosslinker. In some embodiments, 2,2-Dimethoy-2-phenylacetophenone (DMPA) or Irgacure can be used as the UV initiator. UV polymerization/crosslinking was performed in a UV chamber. After the completion of UV polymerization/crosslinking, the sample was removed from the UV chamber and the crosslinking of collagen was continued for about 12 more hours.

### Example 3. Method of Making DMT-MM-APS/TMEDA Hydrogel Inlays with Water Content Ranging from about 94% to about 98%

50 mg of collagen powder was weighed into a 2 ml micro-centrifuge tube labeled 1. 450 mg of 2-(N-Morpholino)ethanesulfonic acid (IVIES) buffer pH 2.90 was then added and the tube placed in 5° C. for 7-10 days to hydrate the collagen. Once collagen was completely hydrated, 200 mg of MES buffer pH 2.90, 125 mg of 10% w/w MPC in IVIES buffer pH 2.90 and 4.71 PEGDA, molecular weight (Mw) 575 were added sequentially to the micro centrifuge tube 1. The tube was vortexed after each addition to properly homogenize the mixture. The tube was then centrifuge and placed in 5° C. The following crosslinking/initiating reagents were then prepared; 4% w/w solution of APS in IVIES buffer pH 2.90, 4% w/w solution of TMEDA solution in IVIES buffer pH 2.90, and a 12% solution of DMT-MM in MES buffer pH 2.90. The tube labeled 1 was removed from 5° C. and 12.19 mg TMEDA solution was added and mixture was homogenized by vortexing. 52 mg of DMT-MM solution and 15.63 mg of APS solution were added to the tube and vortexed to properly mix. The mixture was then centrifuged at 15° C. to remove air bubbles before casting in PMMA cavity molds and allowed to polymerize/crosslink for approximately 12 hours at room temperature, in a humidity chamber. After crosslinking the inlay was demolded and washed several times in 1× phosphate buffer saline (PBS) buffer to remove residual reagents.

### Example 4. Method of Making DMT-MM-LAP Hydrogel Inlays with Water Content Ranging from about 92% to about 96%, Inclusive for Comparison Testing

60 mg of collagen powder was weighed into a 2 ml micro-centrifuge tube labeled 1. 440 mg of MES buffer pH 2.90 was then added and the tube placed in 5° C. for 7-10 days to hydrate the collagen. Once collagen was completely hydrated, 410 mg of MES buffer pH 2.90, 150 mg of 10% w/w MPC in MES buffer pH 2.90 and 5.0 PEGDA, Mw 575 were added sequentially to the micro centrifuge tube 1. The tube was vortexed after each addition to properly homogenize the mixture. The tube was then centrifuge and placed in 5° C. The following crosslinking/initiating reagents were then prepared. 0.25% w/w solution of LAP in IVIES buffer pH 2.90, and a 12% solution of DMT-MM also in MES buffer pH 2.90. The tube labeled 1 was removed from 5° C. and 62.26 mg of DMT-MM solution and 120.0 mg of LAP solution were added to the tube and vortexed to properly mix. The mixture was then centrifuged at 15° C. to remove air bubbles before casting in PMMA cavity molds. The molds were placed in a UV chamber for 30 minutes and then in a humidified chamber for approximately 12 hours at room temperature. After polymerization/crosslinking the inlay was demolded and washed several times in 1×PBS buffer to remove residual reagents.

### Example 5. Method of Making Hydrogel Inlays with Water Content Ranging from about 78% to about 92%, Inclusive

43.33 mg of 12% w/w collagen hydrated in MES buffer pH=2.90 was weighed into a 2 ml micro-centrifuge tube labeled 1. 340 mg of MES buffer pH=2.90, 25 mg MPC, and 50 mg PEGDA (Mw=700). The tube was then vortexed to homogenized and the pH checked and adjusted to between 2.8 and 3.8 with 1 N 0.5:1. (HCl) and 1 N sodium hydroxide (NaOH) solutions. The mixture was then placed in 5° C. The following crosslinking/initiating reagents were then prepared. 0.15% w/w solution of LAP in IVIES buffer pH 2.90, and a 12% solution of DMT-MM also in IVIES buffer pH 2.90. The tube labeled 1 was removed from 5° C. and 5.41 mg of DMT-MM solution and 50.0 mg of LAP solution were added to the tube and vortexed to properly mix. The mixture was then centrifuged at 15° C. to remove air bubbles before casting in PMMA cavity molds. The molds were placed in a UV chamber for 30 minutes and then in a humidified chamber for approximately 12 hours at room temperature. After polymerization/crosslinking the inlay was demolded and washed several times in 1×PBS buffer to remove residual reagents.

### Test Methods

Mechanical Properties

Mechanical properties of the corneal inlays were determined by means of profilometry-based indentation e.g., burst strength measurements following ASTM Standard F2392-04. For the indentation measurements, samples of about 2-6 mm in diameter were tested in 1×PBS in glass vials under spherical indentation in order to obtain the profilometry of the sample. Young's Modulus was subsequently obtained from the resulting profile of the sample.

Water Content

To determine water content of a sample of hydrogel, excess water from a fully hydrated 10 mm diameter disc with a thickness of ˜300 μm is thoroughly blotted with the aid of a KimWipe. The weight of the material, W₁ is taken by placing material on an analytical balance. The inlay is then placed in an oven set at 100° C. for minimum of two and a half hours to completely dry the sample. The weight of the dried sample is measured and recorded as W₂. The water content % WC recorded as a percentage is calculated as:

$$\% WC = ( \frac{{W}_{1} - {W}_{2}}{{W}_{1}} ) \times 100$$

### Example 6. Evaluating Biocompatibility of Inlay Material In Vitro

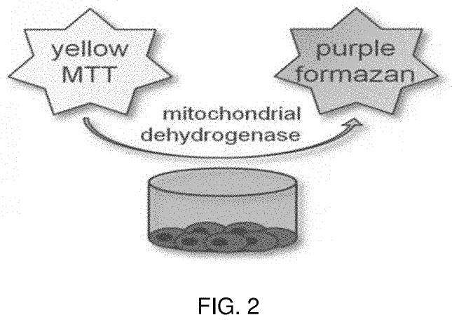

(1) MTT Assay to Quantify Cell Viability on Different Material Samples. (FIG. 2)

Rationale: MTT is used to measure cellular metabolic activity as an indicator of cell viability, proliferation and cytotoxicity. The darker the solution, the greater the number of viable metabolically active cells.

Protocol: 12 mm discs are added to cover most of the well area of 24 well plates. The following numbers of rabbit corneal fibroblasts were seeded in duplicate per well: 200K, 100K, 50K, 25K, 12.5K, 6.25K. Cells were cultured in standard culture medium. 10 wells correspond to test samples; 6 wells correspond to controls (no discs). An MTT calibration plate is seeded on day 3 with the same cell numbers. The calibration plate is evaluated by microscopy before the assay and confluency estimated. On day 4, water soluble yellow MTT (4, 5-dimethylthiazol-2-yl)-2,5-diphenyltetrazolium bromide) is added to the cultures. MTT is reduced to purple insoluble formazan by mitochondrial dehydrogenase in the mitochondria of viable cells. The formazan is solubilized with detergent and measured spectrophotometrically. Viable cells with active metabolism convert MTT into a purple colored formazan product with an absorbance maximum near 570 nm.

(2) In Vitro Cytophilicity Assay

Rationale: An in vitro cytophilicity assay using rabbit corneal fibroblasts measures migration of rabbit keratocytes onto a test material and their attachment. The test therefore addresses whether the material of the exemplary inlays is toxic to cells and whether cells can attach and grow on the material. If cell coverage on the material is judged acceptable (e.g., confluent, no dead cells, others), then the material is a candidate for a more detailed animal implant study. All materials passing this test have shown excellent biocompatibility in subsequent in vivo animal studies in rabbit eyes.

Protocol: Passaged NZW rabbit corneal fibroblasts were seeded in media containing test article. Cell growth was monitored for up to seven days to see if cells attach and grow on the test article, if cell morphology is altered in the presence of the test article, and to measure the thickness of test article. Fully biocompatible test articles showed 100% cell confluence between four and seven days. When implanted in animals, these materials remained clear and transparent even after two years. Test articles that were not biocompatible showed less than 30% cell confluence after seven days. In some cases, no cell growth was observed. When implanted in animals, these materials became hazy between three to six months.

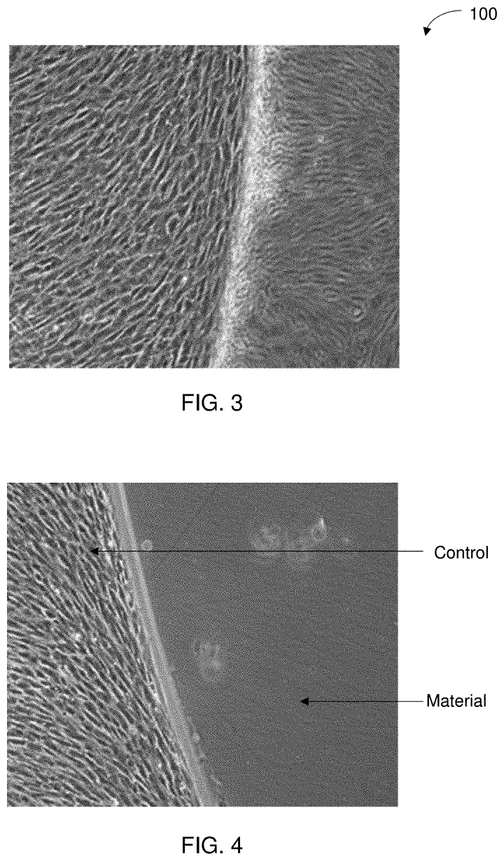

FIG. 3 shows an image of cell coverage on a biocompatible material, and FIG. 4 shows an image of cell coverage on a non-biocompatible material. The whitish or light-colored line in the images is the edge of material on the wall plate which serves as the control. By day seven, cells were confluent on both material and control as seen in FIG. 3. While cells were confluent on the control in FIG. 4, no cells were found on the material.

(3) First Cell Attachment Assay

Sample materials: OM-PC-MPC 1% to 3% collagen.

While these materials passed an in vitro cell growth assay, the materials were found to degrade easily and showed a small degree of haze in in vivo studies. Further tests are to be performed to optimize the material.

### Questions Asked

Can cells grow in the presence of the material/are the materials toxic to cells?
Do cells attach and grow on the materials?
Is cell morphology altered in the presence of material?
What is the thickness of the material samples?

Experimental Protocol

Microscopy Evaluation on Days 4, and 7; thickness—evaluated by microscopy; done in 6 well plates;

Materials: Ferentis secondary and non-secondary; and OM PC-MPC. The OM-PC-MPC materials included a 1% collagen sample and a 2.5% collagen sample, each having water content of about 79-82% inclusive.

Cells: Rabbit corneal fibroblasts, passage 4.

Table 2 is a list of samples tested in the cell attachment assay, including a Group ID, a description, and the number of samples/size. The samples included (1) Nippon 07.14.20, 07.24.20 DMTMM 10%; (2) Nippi 07.24.20, 08.11.20 DMTMM 10%; (3) Nippi 07.24.20, 08.17.20 DMTMM 12%; (4) Nippi 07.24.20, 08.19.20 DMTMM 15%; and (5) Ferentis 1823B, Ferentis 1837A.

TABLE 2
Samples for cell attachment assay

| | | Number of |
|---|---|---|
| Group-ID | Description | Samples/Size |
| Nippon 07.14.20 | New OM PC-MPC stored | 3/8 mm |
| 07.24.20 | in CHCl3/PBS, rinsed | |
| DMTMM 10% | with fresh PBS - placed | |
| | in 6-well plate | |
| Nippi 07.24.20 | New OM PC-MPC stored | 3/8 mm |
| 08.11.20 | in CHCl3/PBS, rinsed | |
| DMTMM 10% | with fresh PBS - placed | |
| | in 6-well plate | |
| Nippi 08.24.20 | New OM PC-MPC stored | 3/8 mm |
| 08.17.20 | in CHCl3/PBS, rinsed | |
| DMTMM 12% | with fresh PBS - placed | |
| | in 6-well plate | |
| Nippi 07.24.20 | New OM PC-MPC stored | 3/8 mm |
| 08.19.20 | in CHCl3/PBS, rinsed | |
| DMTMM 15% | with fresh PBS - placed | |
| | in 6-well plate | |
| Ferentis 1823B | No secondary crosslinking | 3/8 mm |
| | Thickness = 70 μm | |
| Ferentis 1837A | Secondary crosslinking | 3/8 mm |
| | Thickness = 60 μm | |

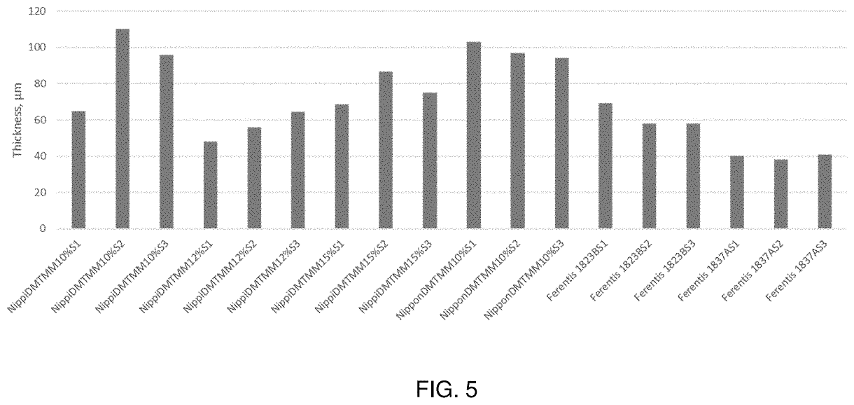

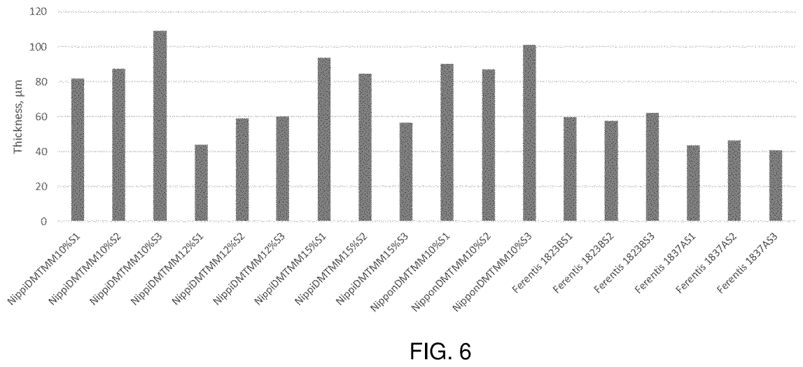

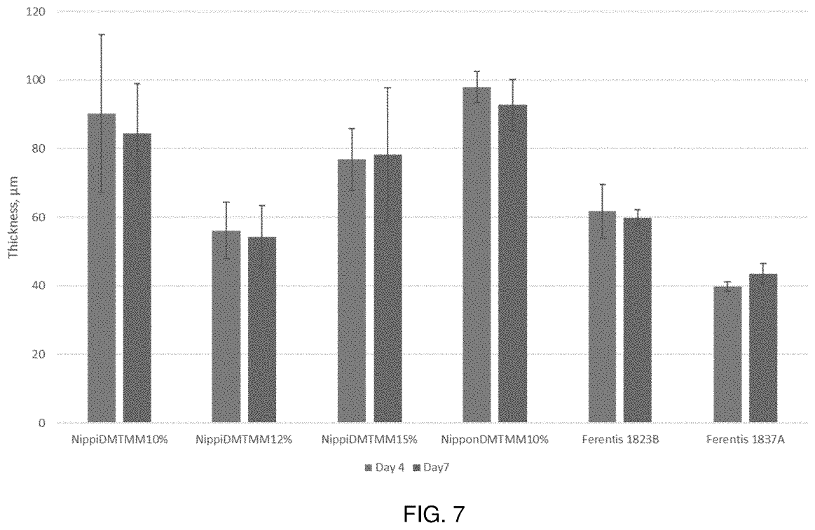

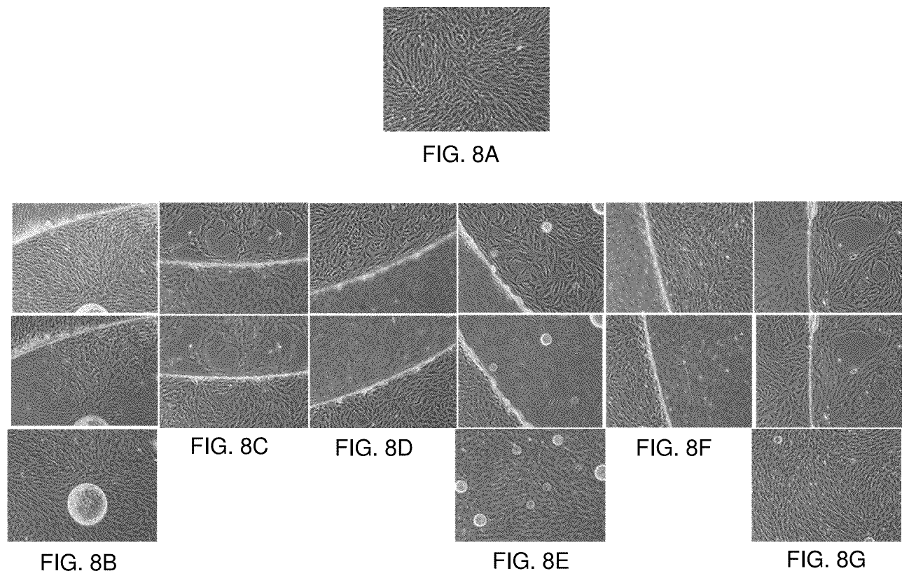

FIGS. 5 and 6 show the measured thickness of the samples based on cell growth at days 4 and 7, respectively, and FIG. 7 shows the measured thickness over time for each of the samples. Tables 3 and 4 show confluency and additional details regarding cell growth for each of the samples based on microscopy imaging at days 4 and 7, respectively. FIGS. 8A-8G and 9A-9G are microscopy images for days 4 and 7, with FIGS. 8A and 9A showing an image of the control, FIGS. 8B and 9B showing images for Nippi 10%, FIGS. 8C and 9C showing images for Nippi 12%, FIGS. 8D and 9D showing images for Nippi 15%, FIGS. 8E and 9E showing images or Nippon 10%, FIGS. 8F and 9F showing images for Ferentis 1823B, and FIGS. 8G and 9G showing images for Ferentis 1837A. The first and second row images of FIGS. 8B-8G and 9B-9G are general microscopy images of the samples, and the third row of images of FIGS. 8B, 8E, 8G and 9B-9E show bubbles formed.

TABLE 3
Microscopy results for different samples
tested in cell attachment assay at day 4.

| Sample | Description |
|---|---|
| Controls | Confluent/overconfluent |
| Nippi 10% | Confluent on and off material |
| Ferentis 1823B | Overconfluent on and off material |
| Nippi 12%, Nippi | Mostly confluent on and off, a |
| 15%, Nippon 10%, | few less confluent spots |
| Ferentis 1837A | |
| Nippi 10%, | Bubble structure sin material - more |
| Nippon 10%, | in Nippon 10% than others. Bubbles |
| Ferentis 1837A | do not move, focal plane is between |
| | the cell on the material and the |
| | cells on the plate |
| Nippi 10%, | Cells growing under |
| Ferentis 1837A | |

TABLE 4
Microscopy results for different samples
tested in cell attachment assay at day 7.

| Sample | Description |
|---|---|
| Controls | Overconfluent, some cells floating |
| Nippon 10%, Nippi 10% | Confluent on and off material |
| Ferentis 1837A, | Overconfluent on and off material |
| Ferentis 1823B | |
| Nippi 12% | A few spots still not totally |
| | confluent on material |
| Nippi 10%, Nippon 10% | Bubble structures in material |
| (a few), Nippi 12% (a few), | |
| Nippi 15% (a few) | |
| Nippon 10%, Nippi 12%, | Cells growing under |
| Ferentis 1837A | |

The cell attachment assay provided the following results. With respect to thickness:
Nippi 10% and Nippon 10% materials are the thickest (85-98 μm).
Nippi 15% are 76-78 μm.
Nippi 12% and Ferentis 1823B are 55-60 μm.
Ferentis 1837A materials are around 40 μm.

Thickness remains steady over culture time.

The cells attached and grew well on all materials, becoming confluent on all samples by day 7 (except one spot on one Nippi 12% sample). Bubbles were observed in the material itself in the following samples:
Nippon 10%, Nippi 10% (a few), and Ferentis 1837A (a few) on day 4.
Nippon 10%, Nippi 10% (a few), Nippi 12% (a few), and Nippi 15% (a few) on Day 7

Second Cell Attachment Assay

### Questions Asked

Can cells grow in the presence of the material/are the materials toxic to cells?
Do cells attach and grow on the materials?
Is cell morphology altered in the presence of material?
What is the thickness of the material samples?

Experimental Protocol

Materials were sterilized either in 0.65% chloroform in 1×PBS or in an antibiotic cocktail in 1×PBS.

Materials were soaked for 20-30 minutes in cell media prior to cell seeding.

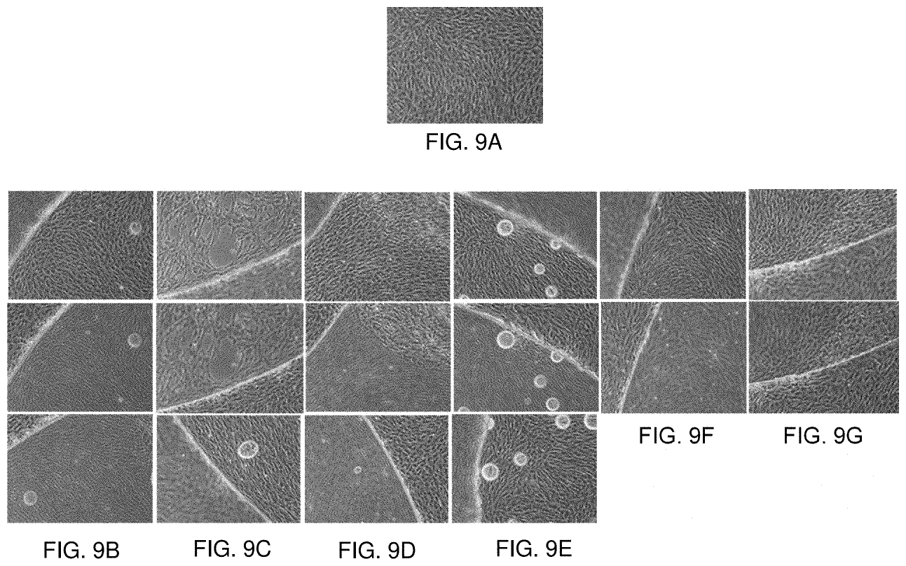

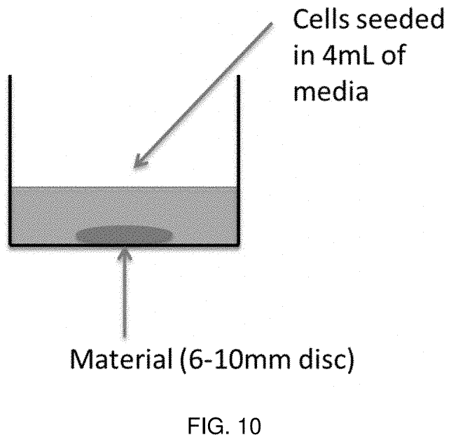

Passaged NZW rabbit corneal keratocytes were seeded at 5000 cells/cm², as shown in FIG. 10 in 6 well plates and incubated. As shown in FIG. 10, cells were seeded in 4 mL of media, and the material was a 6-10 mm disc.

As a control, cells were added to the well plate in the absence of material for each experiment.

Cells were grown for 7 days.

Cells were imaged on Days 4 and 7 using a Nikon Ti100 infrared camera for (1) thickness, and (2) cell attachment and confluency on materials.

Potential modifications to the protocol included Picro Sirius Red staining for collagen content, and evaluation for potential degradation/loss over time in culture. Another potential modification to the protocol includes using Western Blot analysis to evaluate cell activation.

Table 5 provides a summary of the samples for a cell attachment assay. The samples included (1) Nippi 08.25.20 12%, DMTMM 10%-APS 09.16.20; (2) Nippi 08.25.20 12%, DMTMM 12%-APS 09.16.20; (3) Nippi 09.03.20 10%, DMTMM 10%-Lithium 09.17.20; (4) Nippon 07.30.20 10%, DMTMM 10%-Lithium 09.15.20; (5) Ferentis 1842A; (6) SA-13-31B, Non collagen; and (7) SA-13-92A, Collagen 1%.

TABLE 5
Samples for cell attachment assay

| | | | Number of |
|---|---|---|---|
| Group-ID | Description | | Samples/Size |
| Nippi 08.25.20 12% | New OM PC-MPC stored | | 3/8 mm |
| DMTMM 10% - | in CHCl3/PBS, rinsed | | |
| APS 09.16.20 | with fresh PBS - placed | | |
| | in 6-well plate | | |
| Nippi 08.25.20 12% | New OM PC-MPC stored | | 3/8 mm |
| DMTMM 12% - | in CHCl3/PBS, rinsed | | |
| APS 09.16.20 | with fresh PBS - placed | | |
| | in 6-well plate | | |
| Nippi 09.03.20 10% | New OM PC-MPC stored | | 3/8 mm |
| DMTMM 10%- | in CHCl3/PBS, rinsed | | |
| Lithium 09.17.20 | with fresh PBS - placed | | |
| | in 6-well plate | | |
| Nippon 07.30.20 10% | New OM PC-MPC stored | | 3/8 mm |
| DMTMM 10%- | in CHCl3/PBS, rinsed | | |
| Lithium 09.15.20 | with fresh PBS - placed | | |
| | in 6-well plate | | |
| Ferentis 1842A | 300 | μm | 3/8 mm |
| SA-13-31B | >200 | μm | 3/8 mm |
| Non-collagen | | | |
| SA-13-92A | >700 | μm | 3/8 mm |
| Collagen 1% | | | |

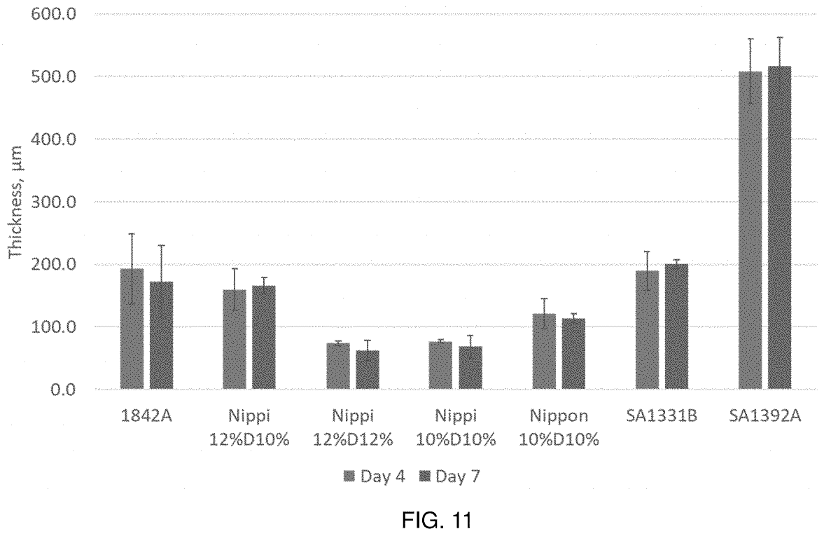

FIG. 11 is a bar graph showing thickness over time for different samples tested in the cell attachment assay at days 4 and 7 is provided.

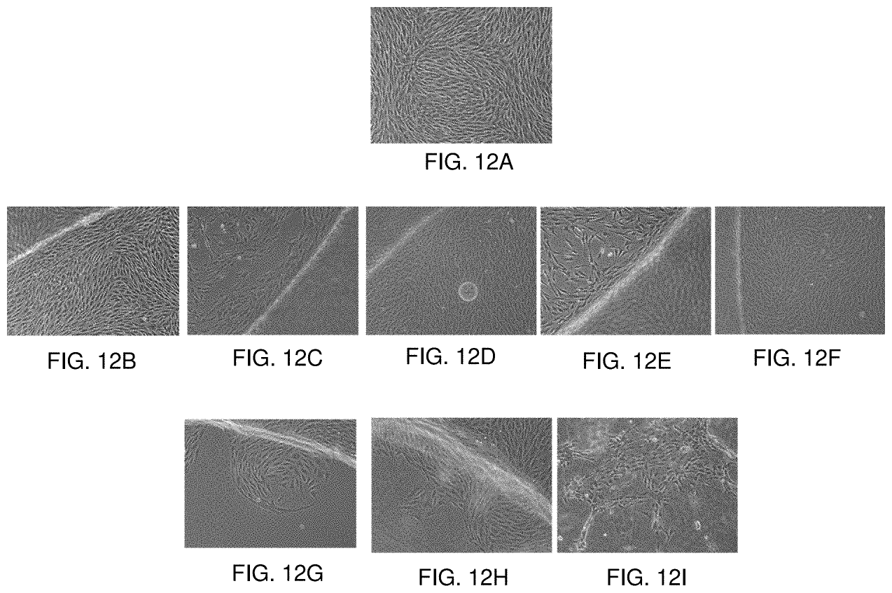

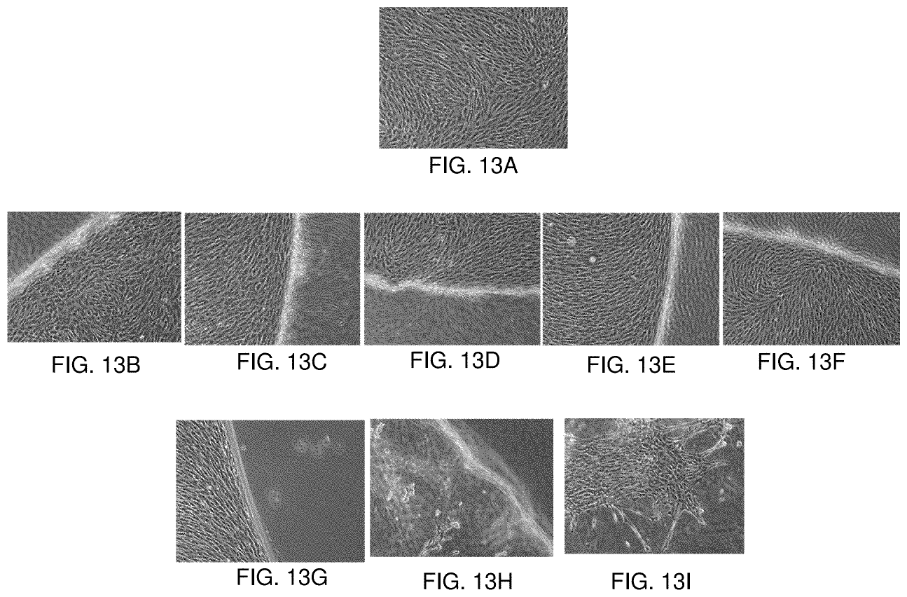

Tables 6 and 7 microscopy results for different samples tested in the cell attachment assay at days 4 and 7, respectively, are provided. The tables include descriptions related to confluency of the cells. FIGS. 12A-12I and FIG. 13A-13I show microscopy images for different samples tested in the cell attachment assay at days 4 and 7, respectively. FIG. 12A and FIG. 13A show images for the control, FIG. 12B and FIG. 13B show images for Ferentis 1842A, FIG. 12C and FIG. 13C show images for Nippi 12% D12%, FIG. 12D and FIG. 13D show images for Nippi 10% D10%, FIG. 12E and FIG. 13E show images for Nippi 12% D10%; FIG. 12F and FIG. 13F show images for Nippon 10%, FIG. 12G and FIG. 13G show images for SA-13-31B, FIG. 1211 and FIG. 1311 show images for SA-13-92A edge, and FIG. 12I and FIG. 131 show images for SA-13-92A on sample.

TABLE 6
Microscopy results for different samples
tested in cell attachment assay at day 4.

| Sample | Description |
|---|---|
| Controls | Confluent |
| Ferentis 1842A | Confluent on and off material, some |
| | cells under, one sample floating |
| Nippi 10% D10% | Confluent on and off material |
| Nippon 10% D10% | Confluent on and off material, |
| | bubbles in material |
| Nippi 12% D10%, | Mostly confluent on and off, some less |
| Nippi 12% D12% | confluent spots, some bubbles in material |
| SA1392A | A few cells on material, some dead |
| | cells, confluent on plate |
| S1331B | No cells on material, confluent on plate |

TABLE 7
Microscopy results for different samples
tested in cell attachment assay at day 7.

| Sample | Description |
|---|---|
| Controls | Overconfluent |
| Ferentis 1842A | Confluent on and off material, some |
| | cells under, one sample floating |
| Nippi10% D10% | Confluent on and off material |
| Nippon 10% D10% | Confluent on and off material, bubbles |
| | in material; material appears grainy |
| Nippi 12% D10%, | Confluent on and off, bubbles in material |
| Nippi 12% D12% | |
| SA1392A | A few cell patches on material, some dead |
| | cells, confluent on plate, cells under |
| S1331B | No cells on material, confluent |
| | on plate, dead cells |

### Summary of Assay

Thickness finding were as follows:
Nippi 10% D10% and Nippi 12%12% are thinnest (65-80 μm).
Nippon 10% D10% are 115-120 μm in thickness.
Nippi 12% D10% about are 160 μm in thickness.
Ferentis 1842A and SA-13-31B materials are around 170-200 μm in thickness.
SA-13-92A materials are around 500 μm in thickness.

Thickness remains steady over culture time.

The cells attached and grew well on all materials, becoming confluent on all samples by day 7.

Control non-collagen samples did not support cell growth (but are not toxic to the cells on the plate).

Collagen coated control samples had a few patches of cells attached.

Bubbles were observed in the material itself in: Nippi 12% D10%, Nippi 12% D12%, and Nippon 10% D10%

### Microscopy

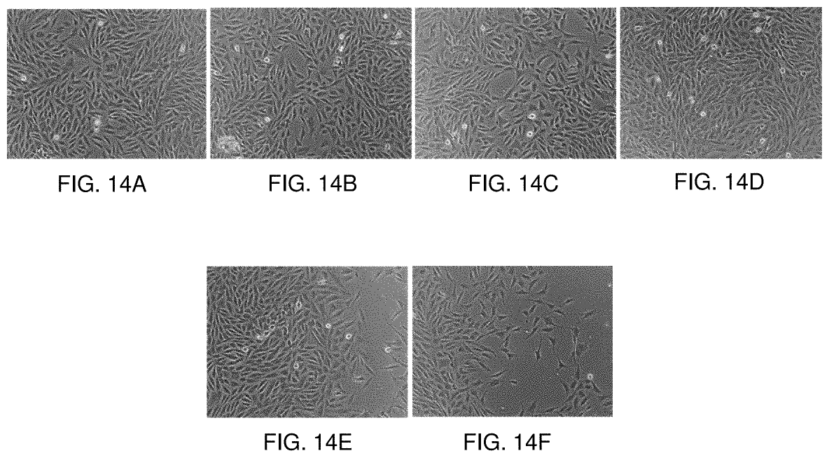

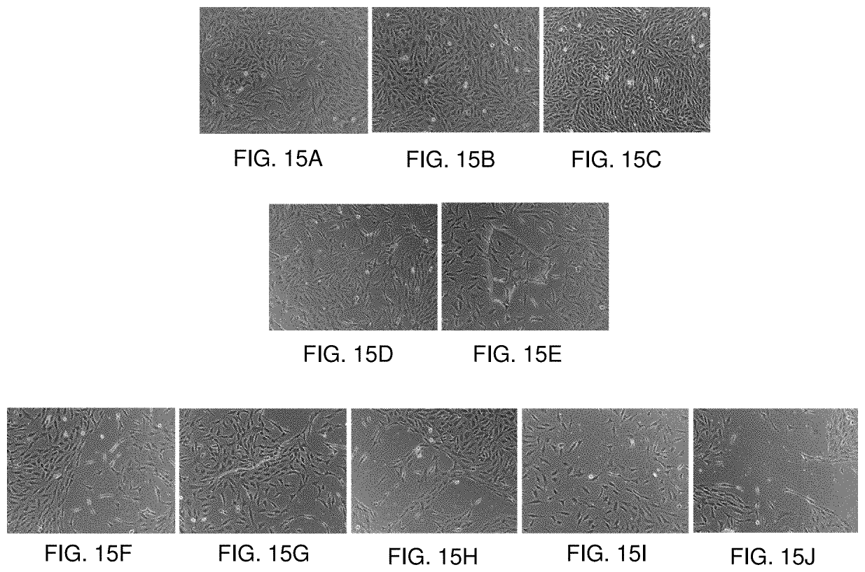

FIGS. 14A-14F show microscopy images for control samples tested in the cell attachment assay. FIGS. 15A-15J show microscopy images for 1745A samples tested in the cell attachment assay. At day 3 microscopy imaging, it was hard to image edges due to 24 well plate. Imaged the center of each well to get an idea of cell growth on the materials compared to controls. Controls included: (1) 4/6 samples 70-100% confluent, and (2) 2/6 samples mostly confluent, a few patches in center. 1746A samples included: (1) 4/10 confluent at edges and nearly confluent in center, (2) 1/10 about 60% confluent in center, confluent at edges, and (3) 5/10 samples 30-40% confluent in center, patchy, some holes. Controls are more confluent overall than samples, but not a stark difference and might be hard to pick up on MTT assay. FIGS. 14A-14D show control sample images for 4/6 samples, 80-100% confluent. FIGS. 14E-14F show control sample images for 2/6 samples mostly confluent, a few patches in center. FIGS. 15A-15C show 1745A sample images for 3/10 confluent at edges and nearly confluent in center. FIGS. 5D-15E show 1745A sample images for 2/10 60-70% confluent in center, confluent at edges. FIGS. 15F-15J show 1745A sample images for 5/10 samples 30-40% confluent in center, patchy, some holes.

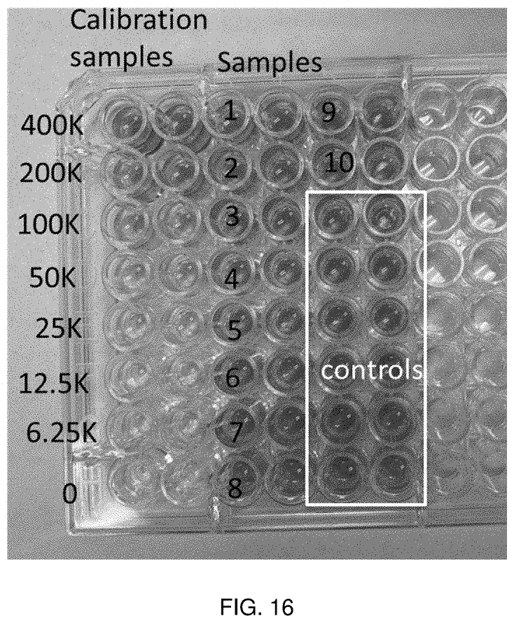

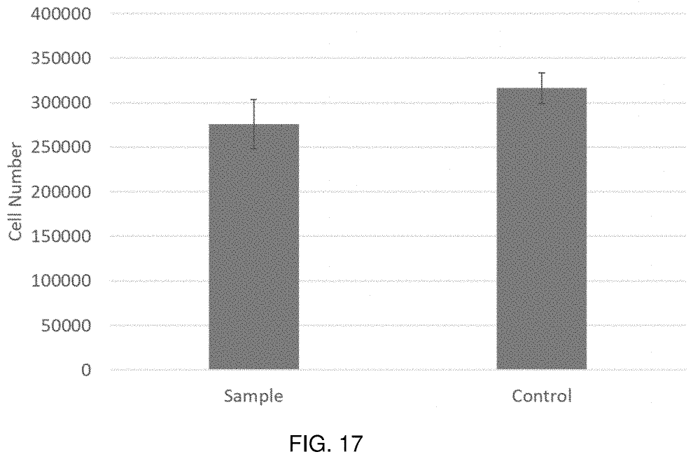

FIG. 16 is an image of an MTT plate illustrating the setup for samples tested in the cell attachment assay. FIG. 17 is a bar graph showing cell numbers for MTT results in the cell attachment assay for a sample and control.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A periodontal implant configured into a physical form selected from a film, a fiber, a filament, a sheet, a thread, a cylindrical implant, an asymmetrically-shaped implant, a fibrous mesh, or an injectable gel, comprising an embedded population of at least 0.5×10*5 live cells;
wherein the implant is fabricated from a hydrogel composition comprising a water content ranging from, e.g., 40% to 92% (w/w inclusive) sufficient to sustain nutritional transport;
wherein the hydrogel composition comprises an interpenetrating polymer network containing a biopolymer and two synthetic polymers, the biopolymer is a collagen; and the synthetic polymers are 2-methacryloyloxyethyl phosphorylcholine (MPC) and poly(ethylene glycol)diacrylate (PEGDA);
wherein the two synthetic polymers are at least partially interlaced on a molecular scale to form a polymer matrix but are not covalently bonded to each other and cannot be separated;
wherein the periodontal implant is highly porous and biodegradable; and
wherein the periodontal implant may support cell growth and permit the transportation of oxygen, nutrients and waste products.

2. The periodontal implant according to claim 1, wherein the periodontal implant is configured into the physical form by molding.

3. The periodontal implant according to claim 1, wherein the injectable gel is capable of being injected with a needle and/or syringe.

4. The periodontal implant according to claim 1, wherein the live cells embedded in the polymer matrix are human mesenchymal stem cells.

5. The periodontal implant according to claim 4, wherein the live human mesenchymal stem cells are derived from peripheral blood, from adipose tissue, or from dental tissue including craniofacial bone, dental pulp, PDL, a dental follicle, tooth germ, apical papilla, oral mucosa, gingival tissue and periosteum of a normal healthy subject.

6. The periodontal implant according to claim 4, wherein
(a) the live human mesenchymal stem cells embedded in the polymer matrix release one or more cell products into the polymer matrix of the implant; and
(b) the cell products are delivered to the periodontium by diffusion.

7. The periodontal implant according to claim 6, wherein the cell products include:
(a) one or more growth factors, fragments or variants thereof;
(b) extracellular vesicles (EVs) comprising a cargo; or
(c) both growth factors, fragments or variants thereof and EVs comprising a cargo.

8. The periodontal implant according to claim 7, wherein the one or more growth factors, fragments or variants thereof, cargo, or both growth factors, fragments or variants thereof and EVs comprising a cargo include one or more of epidermal growth factor (EGF), fibroblast growth factor (FGF), insulin-like growth factor (IGF), platelet derived growth factor (PDGF), transforming growth factor beta (TGFβ), bone morphogenetic proteins (BMPs), and vascular endothelial growth factor (VEGF).

9. The periodontal implant according to claim 1,
a. wherein delivery of the formed periodontal implant comprising the polymer matrix is by surgical placement of the implant at the gum line of a site affected by periodontitis;
b. wherein the population of live cells embedded in the polymer matrix may release one or more cell products into the polymer matrix by diffusion, chemical reaction or both; and
c. wherein wound healing by the released cell products may be by a paracrine effect.

10. The periodontal implant according to claim 9, wherein at least one surface of the implant once implanted is in contact communication with a affected site.

11. The periodontal implant according to claim 10, wherein the embedded population of cells is within 0.400 mm to 0.700 mm, inclusive, of a surface of the implant that is in contact communication with the affected site.

12. The periodontal implant according to claim 10, wherein a surface of the implant, the affected site, or both is modified to promote its adhesion at the affected site by application of a peptide to the surface of the implant, the affected site, or both.

13. The periodontal implant according to claim 12, wherein the peptide is one of amino acid sequence arginine-glycine-aspartic acid (RGD) derived from an ECM protein arginine-glutamic acid-aspartic acid-valine (REDV) derived from fibronectin; tyrosine-isoleucine-glycine-serine-arginine (YIGSR) derived from laminin; or isoleucine-lysine-valine-alanine-valine (IKVAV) derived from laminin.

14. The periodontal implant according to claim 1, wherein the hydrogel composition comprises at least 1%, at least 2%, at least 3%, at least 4%, or at least 5% by weight of the collagen.

15. The periodontal implant according to claim 1, wherein:
(a) a weight ratio of collagen: PEGDA ranges from about 1:3 to about 1:10, inclusive; and
(b) a weight ratio of PEGDA/MPC ranges from 1:0.5 to 0.05:1.

16. The periodontal implant according to claim 1, wherein the collagen is a natural collagen, a synthetic collagen, a recombinant collagen, or a collagen mimic.

17. The periodontal implant according to claim 1, wherein the fibrous mesh is in the form of a woven or nonwoven material.

18. The periodontal implant according to claim 17, wherein the fibrous mesh is in the form of a felt, a gauze, or a sponge.

19. The periodontal implant according to claim 1, wherein the hydrogel polymer matrix is supplemented with growth factors or their biologically active fragments or variants, EVs or both.

20. A method for treating a site affected by periodontal disease comprising delivering locally by implant to an affected site an implant comprising an embedded population of at least 0.5×10*5 live cells;
wherein the implant is fabricated from a hydrogel composition comprising a water content ranging from, e.g., 40% to 92% (w/w inclusive) sufficient to sustain nutritional transport;
wherein the hydrogel composition comprises an interpenetrating polymer network containing a biopolymer and two synthetic polymers, the biopolymer is a collagen; and the synthetic polymers are 2-methacryloyloxyethyl phosphorylcholine (MPC) and poly(ethylene glycol)diacrylate (PEGDA); and
wherein the two synthetic polymers are at least partially interlaced on a molecular scale to form a polymer matrix but are not covalently bonded to each other and cannot be separated;
wherein the periodontal implant is highly porous and biodegradable;
wherein the periodontal implant may support cell growth and permit the transportation of oxygen, nutrients and waste products; and
wherein the periodontal implant may effect wound healing of the affected site.

21. The method of claim 20,
a. wherein delivery of the formed periodontal implant comprising the polymer matrix is by surgical placement of the implant at the gum line of a site affected by periodontitis; and
b. wherein the population of live cells embedded in the polymer matrix may release one or more cell products into the polymer matrix by diffusion, chemical reaction or both; and
c. wherein the cell products are delivered to the periodontium by diffusion.

22. The method of claim 21, further comprising configuring the implant into a physical form selected from a film, a fiber, a filament, a sheet, a thread, a cylindrical implant, an asymmetrically-shaped implant or a fibrous mesh.

23. The method of claim 22, wherein the configuring of the implant into the physical form is by molding.

24. The method of claim 22, wherein the fibrous mesh is in the form of a woven or nonwoven material.

25. The method of claim 24, wherein the fibrous mesh is in the form of a felt, a gauze, or a sponge.

26. The method of claim 20, further comprising contacting at least one surface of the implant once implanted with the affected site; wherein the embedded population of cells is within 0.400 mm to 0.700 mm, inclusive, of a surface of the implant that is in contact communication with the affected site.

27. The method of claim 26, further comprising modifying a surface of the implant, the affected site, or both to promote its adhesion at the affected site by applying a peptide to the surface of the implant, the affected site, or both.

28. The method of claim 27, wherein the peptide is one of amino acid sequence arginine-glycine-aspartic acid (RGD) derived from an ECM protein arginine-glutamic acid-aspartic acid-valine (REDV) derived from fibronectin; tyrosine-isoleucine-glycine-serine-arginine (YIGSR) derived from laminin; or isoleucine-lysine-valine-alanine-valine (IKVAV) derived from laminin.

29. The method of claim 20, wherein the live cells embedded in the polymer matrix are human mesenchymal stem cells.

30. The method of claim 29, wherein the live human mesenchymal stem cells are derived from peripheral blood, from adipose tissue, or from dental tissue including craniofacial bone, dental pulp, PDL, a dental follicle, tooth germ, apical papilla, oral mucosa, gingival tissue and periosteum of a normal healthy subject.

31. The method of claim 29, wherein the live human mesenchymal stem cells embedded in the polymer matrix release one or more cell products into the polymer matrix of the implant.

32. The method of claim 31, wherein the cell products include:
(a) one or more growth factors, fragments or variants thereof;
(b) extracellular vesicles (EVs) comprising a cargo; or
(c) both growth factors, fragments or variants thereof and EVs comprising a cargo.

33. The method of claim 32, wherein the one or more growth factors, fragments or variants thereof, or cargo, or both include one or more of epidermal growth factor (EGF), fibroblast growth factor (FGF), insulin-like growth factor (IGF), platelet derived growth factor (PDGF), transforming growth factor beta (TGFβ), bone morphogenetic proteins (BMPs), and vascular endothelial growth factor (VEGF).

34. The method of claim 20, wherein wound healing of the affected site by the released cell products is by a paracrine effect.

35. The method of claim 20, wherein the hydrogel composition comprises at least 1%, at least 2%, at least 3%, at least 4%, or at least 5% by weight of the collagen.

36. The method according to claim 20, wherein:
(a) a weight ratio of collagen: PEGDA ranges from about 1:3 to about 1:10, inclusive; and
(b) a weight ratio of PEGDA/MPC ranges from 1:0.5 to 0.05:1.

37. The method of claim 20, wherein the collagen is a natural collagen, a synthetic collagen, a recombinant collagen, or a collagen mimic.

38. The method of claim 20, further comprising supplementing the hydrogel polymer matrix in situ with growth factors or their biologically active fragments or variants, EVs or both.
